(19) United States
(12) Patent Application Publication
(10) Pub. No.: US 2025/0375536 A1
(43) Pub. Date: Dec. 11, 2025

(54) VECTOR

(71) Applicant: The University of Bristol, Bristol (GB)

(72) Inventors: Ying Kai Chan, Cambridge, MA (US); David Copland, Bristol (GB); Andrew Dick, Bristol (GB)

(21) Appl. No.: 18/877,936

(22) PCT Filed: Jun. 30, 2023
(86) PCT No.: PCT/GB2023/051731

§ 371 Date: Dec. 20, 2024

Related U.S. Application Data
(60) Provisional application No. 63/357,831, filed on Jul. 1, 2022.

(51) Int. Cl. A61K 48/00; C07K 16/24; C12N 15/86

(52) U.S. Cl. CPC A61K 48/0058; A61K 48/0066; C07K 16/241; C12N 15/86; C07K 2317/55; C12N 2750/14143; C12N 2830/002

(57) ABSTRACT

The present invention provides a vector comprising a nucleotide sequence encoding an anti-TNF antibody or a fragment thereof, wherein the nucleotide sequence encoding the anti-TNF antibody or a fragment thereof is operably linked to an inflammation-inducible promoter.

### FIELD OF THE INVENTION

The present invention relates to vectors for preventing or treating an inflammatory eye disease.

### BACKGROUND TO THE INVENTION

Chronic inflammation in the eye can lead to cumulative damage that eventually causes significant vision loss. Chronic non-infectious uveitis is a sight-threatening intraocular inflammation that accounts for 10% of blindness in the working-age population and has a disproportionately large economic burden (see e.g. Joltikov, K. A. and Lobo-Chan, A. M., 2021. Frontiers in Medicine, 8:695904). Uveitis may include intraocular inflammation that affects the uvea and adjacent structures, such as the cornea, vitreous humor, retina, and optic nerve. Most commonly, uveitis is idiopathic, but can be linked to infection, malignancy, or underlying inflammatory conditions such as spondyloarthritis, sarcoidosis, juvenile idiopathic arthritis (JIA), inflammatory bowel disease, rheumatoid arthritis, tubulointerstitial nephritis, and other autoinflammatory diseases (see e.g. Rosenbaum, J. T., et al., 2019. Seminars in Arthritis and Rheumatism, 49 (3), pp. 438-445).

Currently, the first line treatment for non-infectious uveitis is corticosteroids, which can be administered topically, periocularly, intraocularly, or systemically. However, there are issues associated with this treatment option. Systemic administration of corticosteroids has a number of well-known side effects that can lead to adverse events, and while local administration of corticosteroids can reduce the concentrations required, there is a need for repeat injections as the ocular concentration of the drug declines over time. Due to the recurrent nature of the condition, patients may need to be maintained on continual systemic corticosteroids treatment, which can lead to numerous adverse effects (see e.g. Valenzuela, R. A., et al., 2020. Frontiers in Pharmacology, 11:655). Local treatment options, including intraocular steroid-based implants and intravitreal injection, have not significantly improved the clinical landscape. Whilst effective at reducing recurrence in milder disease, these are associated with significant adverse events, such as cataracts and glaucoma.

Immunosuppressant therapy (IMT) is an alternative to corticosteroid therapy, including antimetabolites, calcineurinic inhibitors, and alkylating agents. When conventional corticosteroids and IMT fail, biological agents and biologics such as TNF inhibitors, IL-1 blockers, and anti-CD20 may be used. However, these agents are associated with adverse events. For example, adverse effects associated with TNF inhibitors include development of autoimmune diseases, increased risk of infection, reactions at the injection site, increased risk of malignancy and worsening of demyelinating disorders (see e.g. Valenzuela, R. A., et al., 2020. Frontiers in Pharmacology, 11:655).

Thus, there is a demand for new approaches for treating or preventing inflammatory eye diseases, such as uveitis.

### SUMMARY OF THE INVENTION

The present inventors have developed a gene therapy for treating or preventing inflammatory eye diseases, such as uveitis, in which anti-inflammatory TNF inhibitors are delivered to the eye.

The inventors have surprisingly demonstrated that a vector encoding a TNF inhibitor under the control of an inflammation-inducible promoter may allow for inflammation-inducible expression of a TNF inhibitor in the eye. When expression of the TNF inhibitor is coupled to an inflammation-inducible promoter, the gene therapy may therefore provide an adaptable and responsive dose level to prevent or treat inflammatory eye disease. Such a gene therapy may prevent re-occurrence of inflammation and/or maintain inflammation at a sub-clinical level, thereby preventing cumulative damage, whilst reducing the occurrence of adverse events.

In one aspect, the present invention provides a vector comprising a nucleotide sequence encoding a TNF inhibitor.

In preferred embodiments, the TNF inhibitor is an anti-TNF antibody or a fragment thereof. Any suitable anti-TNF antibody or fragment thereof may be used. In some embodiments, the TNF inhibitor is any of adalimumab or a fragment thereof, infliximab or a fragment thereof, golimumab or a fragment thereof, or certolizumab or a fragment thereof. In some embodiments, the TNF inhibitor is adalimumab or a fragment thereof, or infliximab or a fragment thereof.

In some embodiments, the TNF inhibitor is an anti-TNF antibody fragment. Any suitable anti-TNF antibody fragment may be used. In some embodiments, the anti-TNF antibody fragment is an antigen-binding fragment (Fab), a fragment antibody (F(ab′)₂), a single chain antibody (scFv), or a single-domain antibody (sdAb). In some embodiments, the anti-TNF antibody fragment is an antigen-binding fragment (Fab).

In some embodiments, the TNF inhibitor is adalimumab or a fragment thereof. In some embodiments, the TNF inhibitor is an antigen binding fragment (Fab) of adalimumab. In some embodiments, the TNF inhibitor is an anti-TNF antibody or a fragment thereof comprising one or more CDR regions selected from SEQ ID NOs: 1 to 6 or derivatives thereof comprising one amino acid substitution. In some embodiments, the TNF inhibitor is an anti-TNF antibody or a fragment thereof comprising CDR regions HCDR1, HCDR2, HCDR3, LCDR1, LCDR2, LCDR3 comprising or consisting of SEQ ID NOs: 1, 2, 3, 4, 5 and 6 respectively, or derivatives thereof comprising one amino acid substitution. In some embodiments, the TNF inhibitor is an anti-TNF antibody or a fragment thereof comprising a heavy chain comprising or consisting of a sequence with at least 70% identity to SEQ ID NO: 7 and/or a light chain comprising or consisting of a sequence with at least 70% identity to SEQ ID NO: 8.

In some embodiments, the heavy chain is encoded by a nucleotide sequence having at least 70% identity to SEQ ID NO: 47 and/or the light chain is encoded by a nucleotide sequence having at least 70% identity to SEQ ID NO: 48. The nucleotide sequence encoding the heavy chain and the nucleotide sequence encoding the light chain may be connected via a linker sequence. Suitably, the linker sequence encodes a 2A self-cleaving peptide, and/or an enzymatically cleavable peptide motif. In some embodiments, the linker sequence encodes a 2A self-cleaving peptide having at least 70% sequence identity to any of SEQ ID NOs: 55-58. The nucleotide sequence encoding the heavy chain and/or the nucleotide sequence encoding the light chain may each be operably linked to a signal sequence. In some embodiments, the signal sequence encodes a signal peptide selected from any of: a Human Growth Hormone (HGH) signal peptide, an interleukin-2 (IL-2) signal peptide, a CD5 signal peptide, an immunoglobulin Kappa light chain signal peptide, a trypsinogen signal peptide, a serum albumin signal peptide, and a prolactin signal peptide.

In some embodiments, the nucleotide sequence encoding a TNF inhibitor encodes an anti-TNF antibody or a fragment comprising or consisting of a heavy chain comprising or consisting of a sequence with at least 70% identity to SEQ ID NO: 7, optionally a 2A self-cleaving peptide having at least 70% sequence identity to any of SEQ ID NOs: 55-58, and a light chain comprising or consisting of a sequence with at least 70% identity to SEQ ID NO: 8. In some embodiments, the nucleotide sequence encoding a TNF inhibitor encodes an anti-TNF antibody or a fragment comprising or consisting of an amino acid sequence having at least 70% identity to SEQ ID NO: 65.

In some embodiments, the nucleotide sequence encoding a TNF inhibitor comprises or consists of: a nucleotide sequence having at least 70% identity to SEQ ID NO: 47, a nucleotide sequence having at least 70% sequence identity to SEQ ID NO: 59 or 60, and a nucleotide sequence having at least 70% identity to SEQ ID NO: 48. In some embodiments, the nucleotide sequence encoding a TNF inhibitor comprises or consists of a nucleotide sequence having at least 70% identity to SEQ ID NO: 66.

In preferred embodiments, the nucleotide sequence encoding a TNF inhibitor is operably linked to an inflammation-inducible promoter. Any suitable inflammation-inducible promoter may be used. Suitably, the inflammation-inducible promoter comprises one or more inflammation-inducible transcription factor binding motif selected from: an AP-1 transcription factor binding motif; a NF-κB transcription factor binding motif; an IRF transcription factor binding motif; a STAT transcription factor binding motif; and a NFAT transcription factor binding motif or any combination thereof.

In some embodiments, the inflammation-inducible promoter comprises one or more AP-1 binding motif and/or one or more NF-κB binding motif. In some embodiments, the inflammation-inducible promoter comprises two or more AP-1 binding motifs and/or two or more NF-κB binding motifs, three or more AP-1 binding motifs and/or three or more NF-κB binding motifs, four or more AP-1 binding motifs and/or four or more NF-κB binding motifs, or five or more AP-1 binding motifs and/or five or more NF-κB binding motifs. In some embodiments, the inflammation-inducible promoter comprises at least one AP-1 binding motif coupled to at least one NF-κB binding motif. In some embodiments, the inflammation-inducible promoter comprises five AP-1 binding motifs coupled to five NF-κB binding motifs. Suitably, an AP-1 binding motif comprises or consists of SEQ ID NO: 70, or comprises or consists of any of SEQ ID NOs: 71-73 or derivatives thereof comprising one nucleotide substitution. Suitably, a NF-κB binding motif comprises or consists of SEQ ID NO: 74, or comprises or consists of SEQ ID NO: 75 or a derivative thereof comprising two or fewer nucleotide substitutions. In some embodiments, the inflammation-inducible promoter comprises or consists of a nucleotide sequence having at least 70% identity to SEQ ID NO: 76.

In some embodiments, the vector comprises a nucleotide sequence having at least 70% identity to SEQ ID NO: 77.

The vector may comprise any other suitable vector elements. The nucleotide sequence encoding the TNF inhibitor may be operably linked to a polyadenylation sequence. Suitably, the polyadenylation sequence is selected from any of: a bovine growth hormone (bGH) polyadenylation sequence, a SV40 polyadenylation sequence, and a rabbit beta-globin polyadenylation sequence. In some embodiments, the polyadenylation sequence comprises or consists of a nucleotide sequence having at least 70% identity to SEQ ID NO: 78. The nucleotide sequence encoding the TNF inhibitor may be operably linked to a woodchuck hepatitis post-transcriptional regulatory element (WPRE). In some embodiments, the WPRE comprises or consists of a nucleotide sequence having at least 70% identity to SEQ ID NO: 79. The nucleotide sequence encoding the TNF inhibitor may be operably linked to an intron. Suitably, the intron is selected from a beta-globin intron or a SV40 intron. In some embodiments, the intron comprises or consists of a nucleotide sequence having at least 70% identity to SEQ ID NO: 80.

In preferred embodiments, the vector is a viral vector. Any suitable viral vector may be used. Suitably, the viral vector is any of a parvoviral vector, preferably an adeno-associated virus (AAV) vector, an adenoviral vector, a herpes simplex viral vector, an anelloviral vector, a retroviral vector or a lentiviral vector.

In preferred embodiments, the vector is an adeno-associated virus (AAV) vector. In preferred embodiments, the vector is an AAV vector particle. The AAV vector particle may be pseudotyped to confer ocular tissue tropism. Suitably, the AAV vector particle comprises AAV2 capsid proteins or AAV2 capsid variant proteins, optionally wherein the AAV2 capsid variant is selected from any of: AAV2.tYF, AAV2.7m8, R100, AAV2.GL and AAV2.NN. The vector may comprise one or more inverted terminal repeats (ITRs).

In some embodiments, the vector comprises or consists of a nucleotide sequence having at least 70% identity to SEQ ID NO: 91.

In one aspect, the present invention provides a vector comprising or consisting of a nucleotide sequence having at least 70% identity to SEQ ID NO: 91. The vector may be a viral vector. The vector may be an AAV vector.

In one aspect, the present invention provides a cell comprising the vector of the present invention. The cell may be an isolated cell.

In one aspect, the present invention provides a kit for the production of the vector of the present invention.

In one aspect, the present invention provides a pharmaceutical composition comprising the vector of the present invention or the cell of the present invention. The vector or cell may be in combination with a pharmaceutically acceptable carrier, diluent or excipient.

In one aspect, the present invention provides a vector according to the present invention, a cell according to the present invention, and/or a pharmaceutical composition according to the present invention, for use as a medicament.

In one aspect, the present invention provides use of a vector according to the present invention, a cell according to the present invention, or a pharmaceutical composition according to the present invention, for the manufacture of a medicament.

In one aspect, the present invention provides a method comprising administering a vector according to the present invention, a cell according to the present invention, or a pharmaceutical composition according to the present invention, to a subject in need thereof.

In one aspect, the present invention provides a vector for use in preventing or treating an inflammatory eye disease, wherein the vector comprises a nucleotide sequence encoding a TNF inhibitor, and wherein the nucleotide sequence encoding the TNF inhibitor is operably linked to an inflammation-inducible promoter.

In one aspect, the present invention provides use of a vector in the manufacture of a medicament for preventing or treating an inflammatory eye disease, wherein the vector comprises a nucleotide sequence encoding a TNF inhibitor, and wherein the nucleotide sequence encoding the TNF inhibitor is operably linked to an inflammation-inducible promoter.

In one aspect, the present invention provides a method for preventing or treating an inflammatory eye disease, wherein the method comprises administering a vector to a subject in need thereof, wherein the vector comprises a nucleotide sequence encoding a TNF inhibitor, and wherein the nucleotide sequence encoding the TNF inhibitor is operably linked to an inflammation-inducible promoter.

In one aspect, the present invention provides a vector according to the present invention, or a pharmaceutical composition according to the present invention, for use in preventing or treating an inflammatory eye disease.

In one aspect, the present invention provides use of a vector according to the present invention, or a pharmaceutical composition according to the present invention, for the manufacture of a medicament for preventing or treating an inflammatory eye disease.

In one aspect, the present invention provides a method of preventing or treating an inflammatory eye disease comprising administering a vector according to the present invention, or a pharmaceutical composition according to the present invention, to a subject in need thereof.

The inflammatory eye disease may be any inflammatory eye disease. Suitably, the inflammatory eye disease is uveitis. The vector or pharmaceutical composition may be administered in response to relapse of an inflammatory eye disease, particularly wherein the inflammatory eye disease is uveitis.

The vector or pharmaceutical composition may be administered by any suitable route. Suitably, the vector or pharmaceutical composition is administered intraocularly. In some embodiments the vector or pharmaceutical composition is administered via intravitreal, subretinal, direct retinal, subconjunctivital, sub-Tenon's or suprachoroidal injection. In some embodiments, the vector or pharmaceutical composition is administered via intravitreal injection.

The vector or pharmaceutical composition may be administered in any suitable regimen. Suitably, the vector or pharmaceutical composition is administered as a single dose. Suitably, the vector is administered at a dose of at least about 1E10 vg/mL, at least about 1E11 vg/mL, at least about 1E12 vg/mL, or at least about 5E12 vg/mL. Suitably, the vector is administered at a dose of at least about 1E9 vg/eye, at least about 1E10 vg/eye, or at least about 1E11 vg/eye. Suitably, the vector is administered at a dose of about 1E9 vg/eye to about 5E12 vg/eye.

### BRIEF DESCRIPTION OF DRAWING

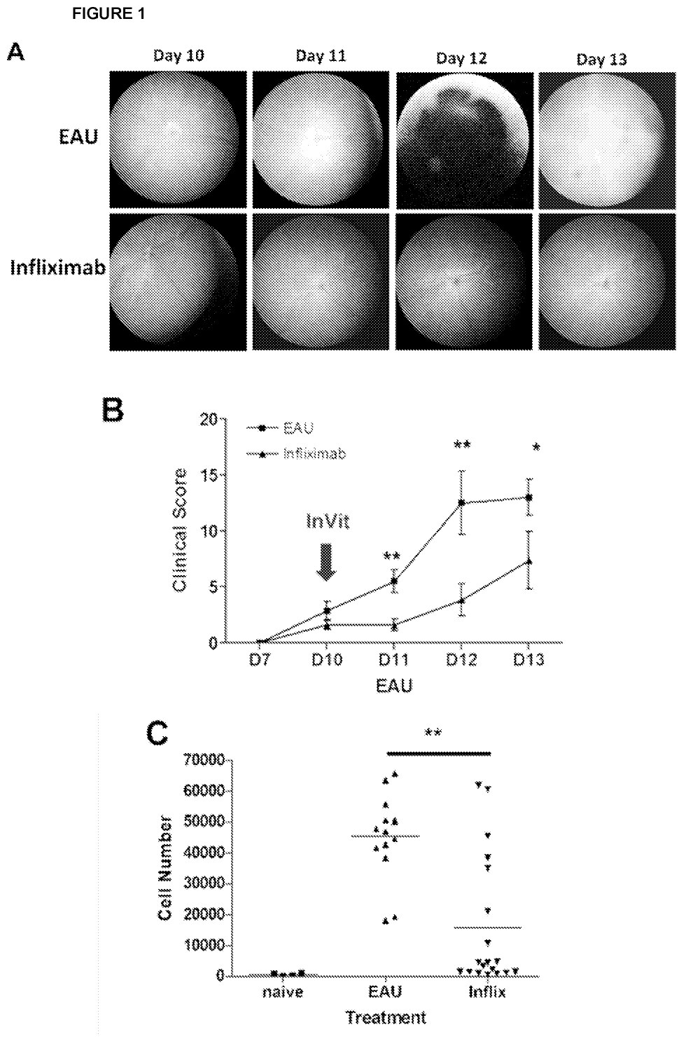

### FIGS. 1A-1C: Local Administration of an Anti-TNF Antibody Suppresses Experimental Autoimmune Uveoretinitis (EAU)

B10.RIII mice were immunized for Experimental Autoimmune Uveoretinitis (EAU) and eyes monitored using Topical Endoscopic Fundal Imaging (TEFI) from day 10 onward to select experimental mice displaying clinically evident disease. Groups of mice were injected via intravitreal route with 15 μg infliximab or vehicle control (EAU) on day 10. Eyes were enucleated (day 14), and retinal infiltrate characterized. (1A) Representative fundus images, (1B) clinical disease scores and (1C) flow cytometric analysis of total CD45+ cell numbers from single eyes at day 14. **P<0.005; Data presented as means+/−SEM, representative of two independent experiments.

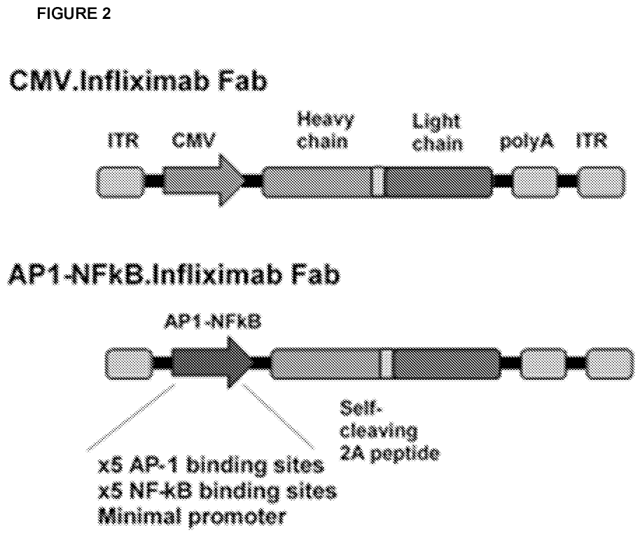

### FIG. 2: Therapeutic Vector Design

Schematic showing the vector organization of CMV. Infliximab Fab (expression under control of a constitutive CMV promoter) and AP1-NFkB. Infliximab Fab (expression under control of an inflammation-inducible promoter comprised of 5 repeated AP1 and NFkB binding sites). The heavy and light chains of Infliximab Fab are separated by a self-cleaving 2A peptide.

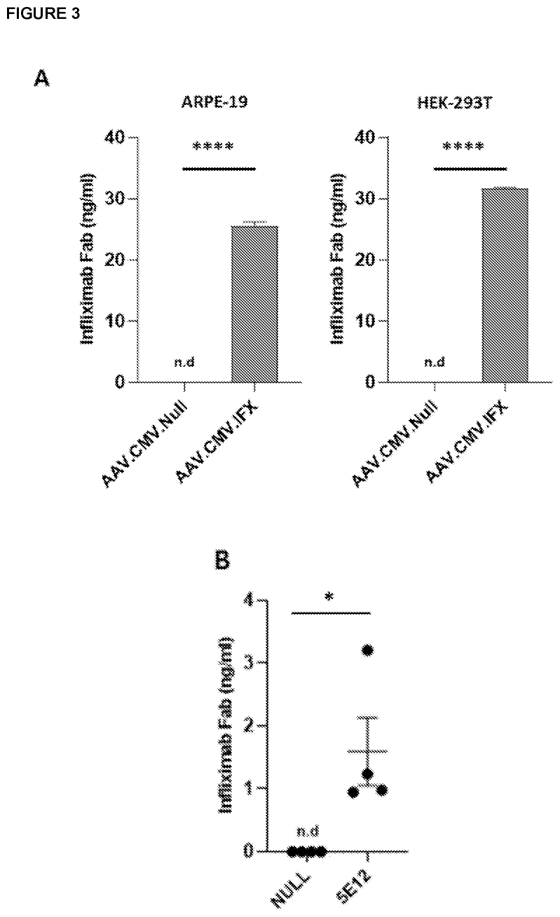

### FIGS. 3A-3B: Constitutive Therapeutic Transgene Expression

(3A) HEK-293T (standard cell line for AAV development) or ARPE-19 (ocular cell line) cells were transduced with AAV2.CMV. Infliximab or AAV.CMV.NULL vectors [MOI 1E5vg/cell], and culture supernatants assayed using a clinical IFX ELISA kit. Detectable expression of Infliximab Fab from both cell types was evident by 72 hrs (˜30 ng/ml). ****P<0.0001. Data presented as means+/−SEM. (3B) AAV2.CMV. Infliximab or AAV.CMV.NULL was administered by intravitreal (IVT) injection at 5E12 vg/ml into eyes of healthy B10.RIII mice. At 4 wks post-AAV, mice were killed, eyes dissected and supernatants (retina and vitreous) assayed using the clinical IFX ELISA kit. Detectable expression of in vivo Infliximab Fab (˜1.5 ng/ml) observed in eyes receiving the 5E12 vg/ml dose. *P<0.05. Data presented as means+/−SEM, with each data point representing a single eye.

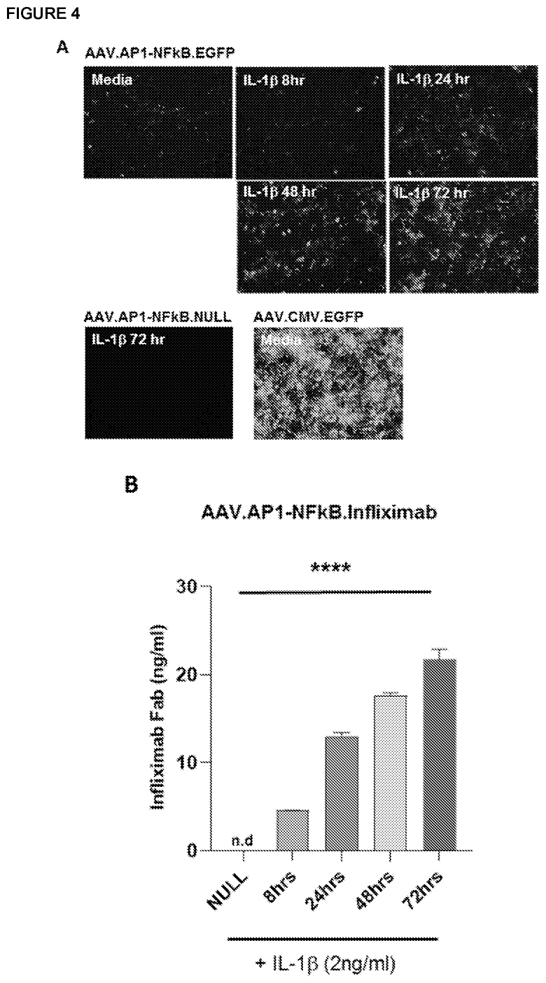

### FIGS. 4A-4B: In Vitro Inducible Transgene Expression

HEK-293T cells transduced with AAV2.AP1-NFkB.EGFP (reporter vector) or AAV2.AP1-NFkB. Infliximab (therapeutic vector) were stimulated with recIL-1b (2 ng/ml). (4A) Activation leads to visible GFP expression at 24 hs, increasing in intensity by 72 hrs, with no GFP signal observed with AAV2.AP1-NFkB.NULL (control vector). Images captured on EVOS FL, 10× magnification. (4B) Stimulation results in a rapid induction of Infliximab Fab expression (8 hrs), accumulation reaching ˜20 ng/ml at 72 hrs. ****P<0.0001; Data presented as means+/−SEM.

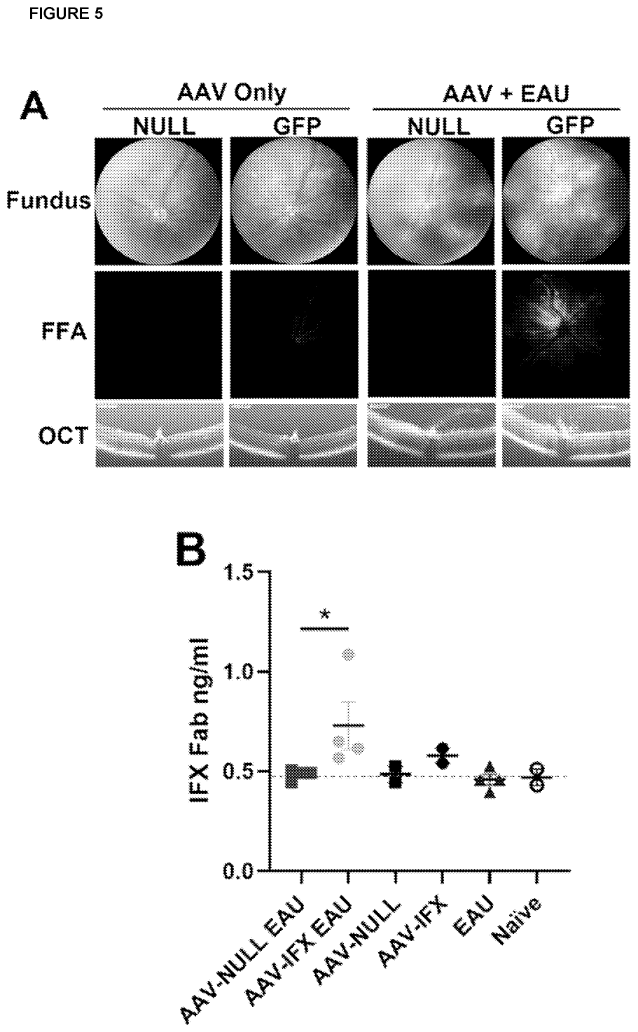

### FIGS. 5A-5B: In Vivo Inducible Transgene Expression

AAV2.AP1-NFkB.EGFP (reporter) or AAV.AP1-NFkB.NULL (control) at a 5E12 vg/ml dose was administered by intravitreal (IVT) injection to C57BL/6J mice. (5A) At 4 wks post-AAV injection, mice were immunized to induce experimental autoimmune uveoretinitis (EAU), and imaged to monitor onset of ocular inflammation. At day 14 EAU, representative fundus and OCT images demonstrate clear clinical signs of disease (perivascular sheathing and vitreous infiltrate), and induction of GFP expression. In mice that only received AAV (no EAU), no clinical signs of disease or expression of the GFP transgene are observed. (5B) Groups of mice were injected with AAV2.AP1-NFkB. Infliximab or AAV2.AP1-NFkB.NULL in the contralateral eye, and EAU induced at 4 wks post-AAV. At 3 wks post-EAU, mice were killed, eyes dissected and supernatants (retina and vitreous) assayed using the clinical IFX ELISA kit. Detectable expression of the Infliximab Fab is only observed in EAU eyes receiving the therapeutic vector and not the control. *P<0.05. Data presented as means+/−SEM, with each data point representing a single eye.

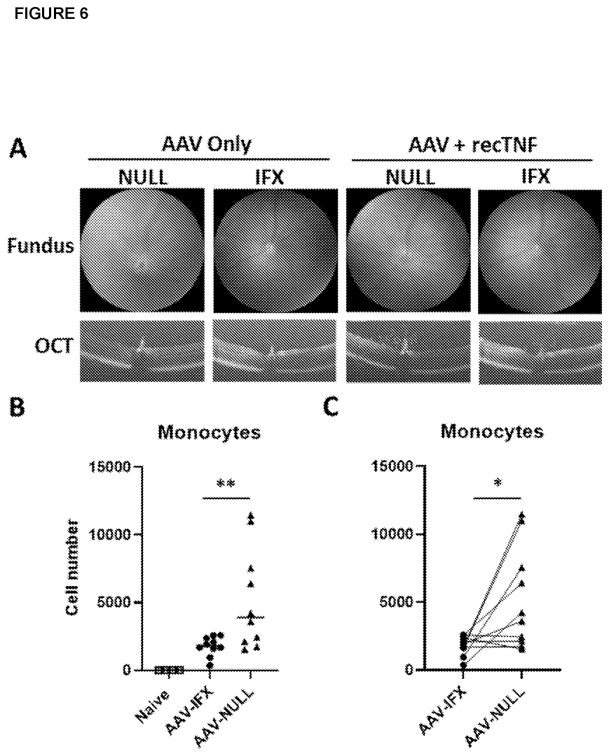

### FIGS. 6A-6C: In Vivo Evaluation of Constitutive Therapeutic Transgene Efficacy

To demonstrate efficacy of the constitutive therapeutic transgene, groups of mice were injected with AAV7m8.CMV. Infliximab or AAV7m8.CMV.NULL in the contralateral eye, followed by intravitreal administration of recombinant human TNF (rec_hTNF) at 4 wks post-AAV. At 18 hrs (peak of inflammatory response to rec_hTNF), representative fundus and OCT infliximab eyes (6A). In mice that only received AAV, no clinical signs of disease are observed. At 18 hrs mice were killed, eyes dissected prepared for flow cytometric analysis to determine absolute numbers of Ly6C+ monocytes (predominant infiltrate in this model) from single eyes. A significant reduction in number of monocytes in eyes receiving the therapeutic vector and not the control (6B), this effect is further highlighted further with paired (contralateral eye) analysis (6C). **Wilcoxon signed rank test; *P<0.05 Wilcoxon matched pairs analysis.

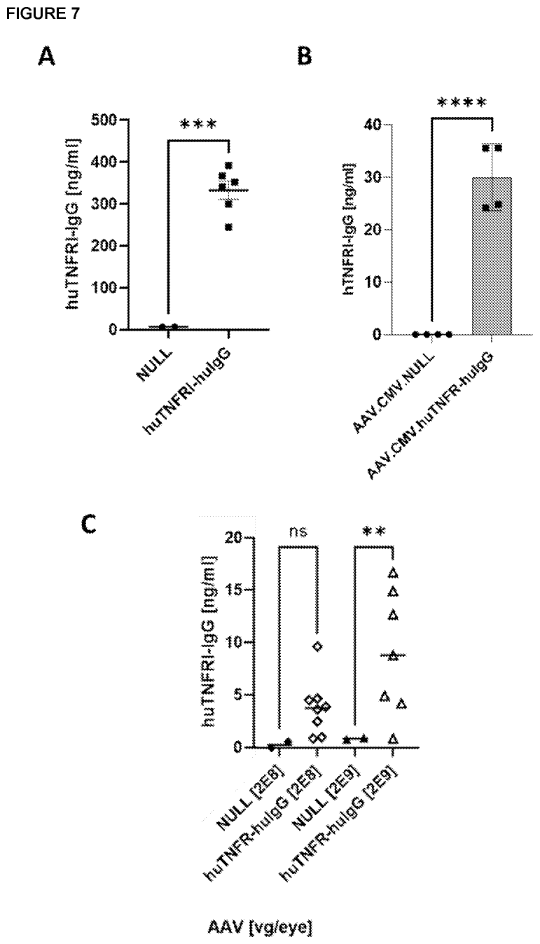

### FIGS. 7A-7C: Constitutive Expression of Other Anti-TNF Biologics In Vitro and In Vivo

HEK-293T cells were transfected with the huTNFRI-huIgG plasmid, and culture supernatants assayed using an anti-human TNF antibody ELISA kit. Detectable expression of huTNFRI-huIgG was evident by 48 hrs (7A). HEK-293T cells were transduced with AAV7m8.CMV.huTNFRI-huIgG or AAV7m8.CMV.NULL vectors [MOI 1E5 vg/cell], and culture supernatants assayed using an anti-human TNF antibody ELISA kit. Detectable expression was evident by 72 hrs (˜30 ng/ml) (7B). ****P<0.0001. Data presented as means+/−SEM. AAV7m8.CMV.huTNFRI-huIgG or AAV7m8.CMV.NULL was administered by intravitreal (IVT) injection at range of doses [2E8 or 2E9 vg/eye] into the eyes of healthy C57BL/6J mice. At 4 wks post-AAV, mice were killed, eyes dissected and supernatants (retina and vitreous) assayed using the anti-human TNF antibody ELISA kit. Detectable expression of huTNFRI-huIgG at ˜4 ng/ml and 9 ng/ml was observed in eyes receiving 2E8 and 2E9 vg/eye doses respectively (7C). ns-not significant; One way ANOVA; **P<0.05. Data presented as means +/− SEM, with each data point representing a single eye.

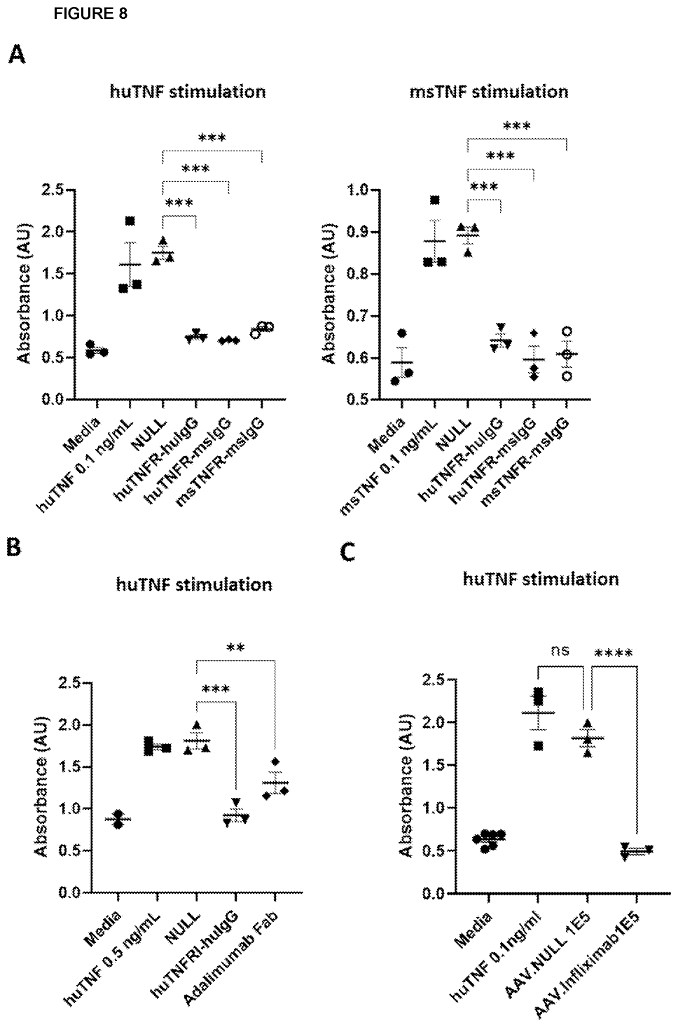

### FIGS. 8A-8C: Bioactivity of Constitutively Expressed Anti-TNF Transgenes In Vitro

HEK-BLUE TNF reporter cells were transfected with AAV.CMV.huTNFRI-huIgG, AAV.CMV.huTNFRI-msIgG, AAV.CMV.msTNFRI-msIgG, AAV.CMV.ADALIMUMAB plasmids or AAV2.CMV. Infliximab for 48 hrs. Cells were then stimulated with recombinant human TNF or mouse TNF (0.5 ng/ml) for further 24 hours and NFkB activation assessed. All the TNFRI antibody-like plasmid constructs inhibited huTNF- and msTNF-mediated activation in the reporter cell line compared to NULL or recTNF alone (8A). For the monoclonal Fab based anti-TNF biologics, plasmid expression of Adalimumab Fab (8B) and AAV-mediated Infliximab Fab expression (8C) both inhibit activation with huTNF.

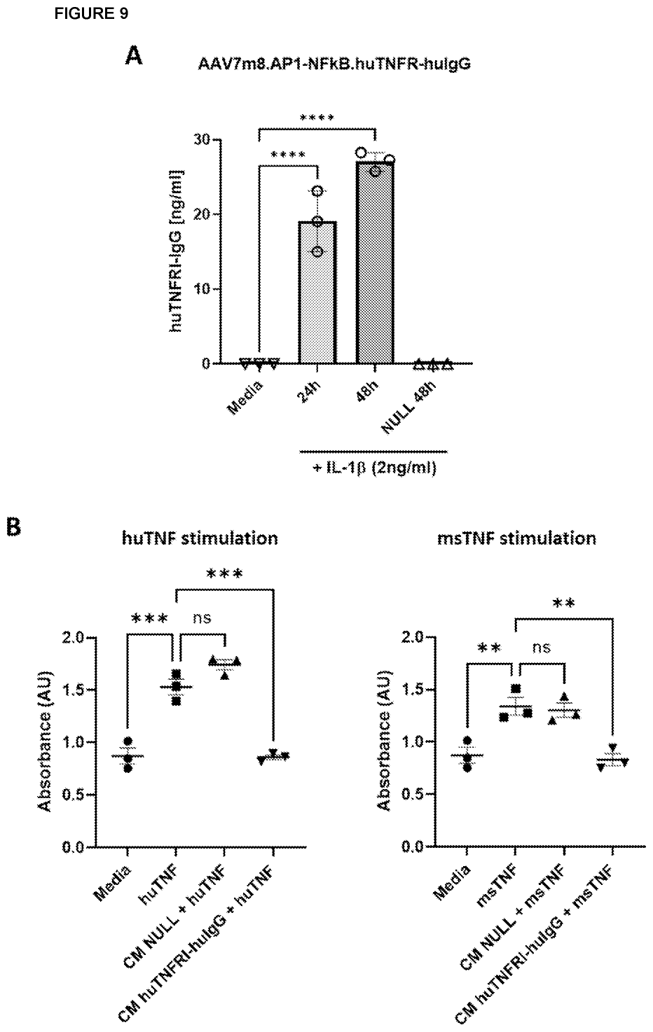

### FIGS. 9A-9B: Evaluation of Expression and Bioactivity of an Inducible Anti-TNF Transgene In Vitro

HEK-293T cells were transduced with AAV7m8.AP1-NFkB.huTNFRI-huIgG or AAV7m8.AP1-NFkB.NULL vectors [MOI 1E5 vg/cell] and stimulated with IL-1b (2 ng/ml). Stimulation results in robust induction of huTNFRI-huIgG expression by 24 hrs, accumulation reaching ˜25 ng/ml at 48 hrs (9A). One way ANOVA; **P<0.0001. Data presented as means+/−SEM. Conditioned media was “spiked” with recombinant human or mouse TNF (final concentration 10 ng/ml), and incubated with HEK-BLUE reporter cells for 24 hrs. In response to both huTNF or mTNF stimulation alone, or with conditioned media from AAV7m8.AP1-NFkB.NULL, NFkB activation is robustly induced in the reporter cells. When supplemented with conditioned media from cells transduced with AAV7m8.AP1-NFkB.huTNFRI-huIgG and stimulated with IL-1b, NFkB activation in the reporter cells is completely suppressed (9B). One way ANOVA; **p<0.05 ***p<0.001; Data presented as means+/−SEM.

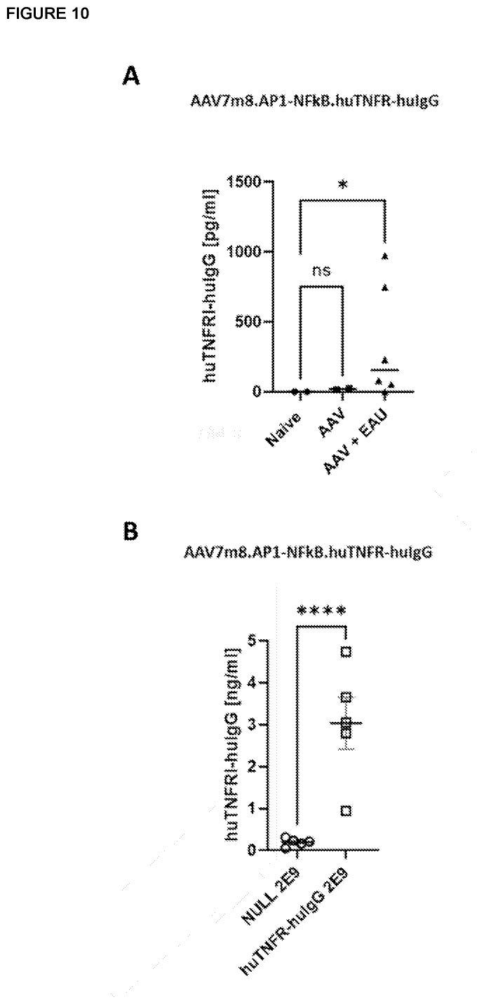

### FIGS. 10A-10B: Evaluation of Expression and Bioactivity of an Inducible Anti-TNF Transgene In Vivo

Mice were injected with AAV7m8.AP1-NFkB.huTNFRI-huIgG, and EAU induced at 4 wks post-AAV. At day 19 post-EAU, when mild to moderate clinical signs of inflammation (not yet peak disease) were observed, mice were killed, eyes dissected and supernatants (retina and vitreous) assayed using the anti-human TNF antibody ELISA. Clinical disease drove detectable expression of the huTNFRI-huIgG in EAU eyes receiving the therapeutic vector and not the control (AAV only) (10A). Mice were injected with AAV7m8.AP1-NFkB.huTNFRI-huIgG and AAV7m8.AP1-NFkB.NULL (contralateral eye control) at 2E9 vg/eye. At 4 wks post-AAV, mice received bilateral administration of recombinant human TNF (rec_hTNF), 18 hrs later were killed, eyes dissected and ocular supernatants (retina and vitreous) assayed for huTNFRI-huIgG expression. Acute activation elicits a significant increase in expression of the huTNFRI-huIgG compared to the control (10B). ****P<0.0001. Data presented as means +/− SEM, with each data point representing a single eye.

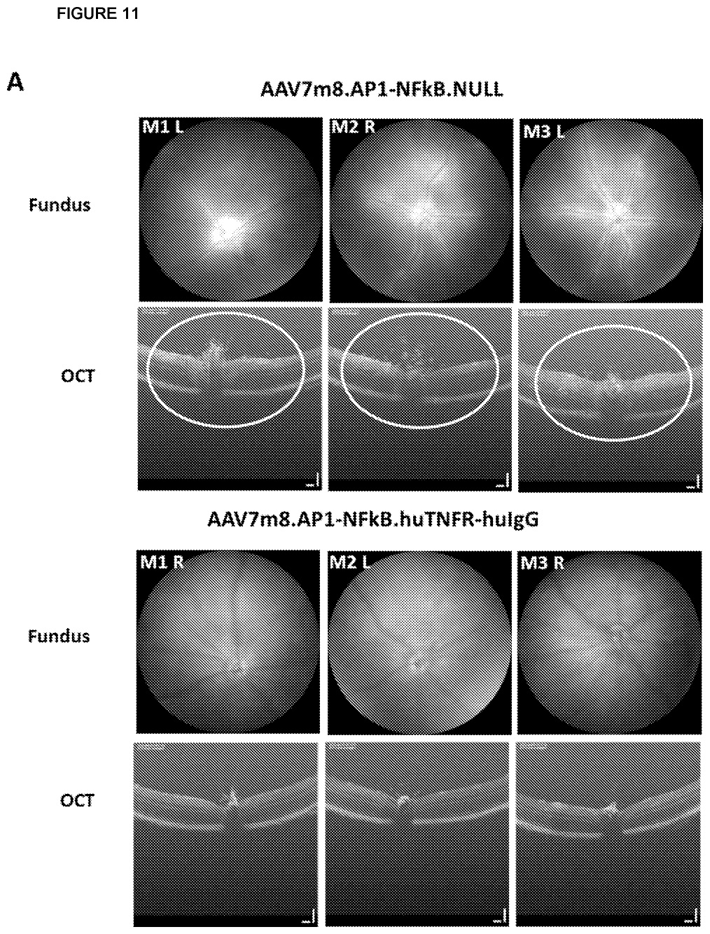

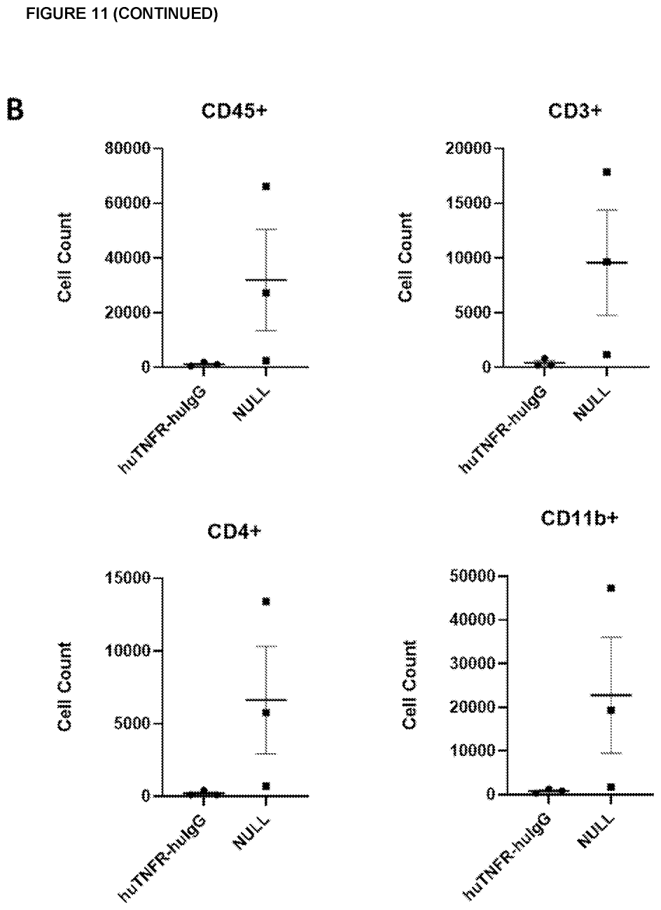

### FIGS. 11A-11B: Evaluation of Inducible Therapeutic Transgene Efficacy In Vivo

B10.RIII mice were injected with AAV7m8.AP1-NFkB.huTNFRI-IgG or AAV7m8.CMV.NULL [2E9 vg/eye] in contralateral eyes, and then immunized for EAU at 4 wks post-AAV. At day 11, representative fundus and OCT images demonstrate increased inflammation in the eyes receiving the control NULL vector (11A). The contralateral eyes of the same three animals, which received the inducible therapeutic vector, showed substantially reduced clinical inflammation, both peri-vascular sheathing and vitreous infiltrate. At this time-point mice were killed, eyes dissected prepared for flow cytometric analysis to determine absolute numbers of CD45+ (all leukocytes), CD3+ (lymphocytes), CD4+ (Th T cells) and CD11b+ (macrophages and monocytes) populations from single eyes (11B). We observed a trend of reduction in immune cell infiltrates with AAV7m8.AP1-NFkB.huTNFRI-IgG compared to AAV7m8.CMV.NULL.

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation and amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto. All publications mentioned in the specification are herein incorporated by reference.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

### TNF Inhibitors

The vectors of the present invention comprise a nucleotide sequence encoding a TNF inhibitor. The present inventors have surprisingly shown that such a vector may be used to prevent or treat an inflammatory eye disease.

As used herein, a “TNF inhibitor” may be any protein that suppresses an inflammatory response to TNF. Tumour necrosis factor (TNF) is also known as cachexin or cachectin, and may also be known as tumour necrosis factor alpha (TNF-α). TNF is synthesized as a transmembrane protein (mTNF) and cleaved to soluble TNF (sTNF). There are two types of TNF, which are very closely related, TNF-alpha and TNF-beta. The activities of both TNFs are mediated through binding to the TNF receptors, TNFR1 and TNFR2. The binding of TNF may activate several signalling pathways, including transcription factor activation, proteases, and protein kinases. This signalling may lead to activation of the target cell leading to the inflammatory and immune response by releasing several cytokines and apoptotic pathway initiation (see Gerriets, V., et al., 2021. “Tumor necrosis factor inhibitors”. In StatPearls).

Example TNF inhibitors include adalimumab, infliximab, golimumab, certolizumab pegol, etanercept, XPro1595, XENP345, R1antTNF, Atrosab, and Atrosimab (see e.g. Lis, K., Kuzawińska, O. and Bałkowiec-Iskra, E., 2014. AMS, 10 (6), p. 1175; and Fischer, R., et al., 2020. Frontiers in cell and developmental biology, 8, p. 401). Suitably, a TNF inhibitor may inhibit TNF activity by directly binding to TNF. For example, a TNF inhibitor may be an anti-TNF antibody or fragment thereof (e.g. adalimumab, infliximab, golimumab, certolizumab), or comprise the TNF-binding domain of a TNFR receptor (e.g. etanercept). Alternatively, a TNF inhibitor may inhibit TNF activity by binding to a TNF receptor. For example, a TNF inhibitor may be a TNF mutein (e.g. XPro1595, XENP345, R1antTNF) or may be an anti-TNFR antibody or fragment thereof (e.g. Atrosab and Atrosimab).

### Anti-TNF Antibody or Fragment Thereof

In preferred embodiments, the TNF inhibitor is an anti-TNF antibody or a fragment thereof.

Antibodies are glycoproteins belonging to the immunoglobulin superfamily. Antibodies are typically made of basic structural units, each with two heavy chains and two light chains. An antibody may recognise an antigen via the fragment antigen-binding (Fab) variable region. The fragment crystallizable region (Fc region) is the tail region of an antibody that may allow antibodies to activate the immune system. The hinge region is a stretch of heavy chains linking the Fab and Fc regions.

“Heavy chain variable region” or “VH” refers to the fragment of the heavy chain of an antibody that contains three CDRs interposed between flanking stretches known as framework regions, which form a scaffold to support the CDRs. “Light chain variable region” or “VL” refers to the fragment of the light chain of an antibody that contains three CDRs interposed between framework regions.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen. The heavy chain variable region and the light chain variable region each contain 3 CDRs (heavy chain CDRs 1, 2 and 3 and light chain CDRs 1, 2 and 3, numbered from the amino to the carboxy terminus).

The CDRs of the variable regions of a heavy and light chain of an antibody can be predicted from the heavy and light chain variable region sequences of the antibody, using prediction software available in the art, e.g. using the Abysis algorithm, or using the IMGT/V-QUEST software (see e.g. Lefranc et al, 2009 NAR 37: D1006-D1012 and Lefranc 2003, Leukemia 17:260-266). CDR regions identified by either algorithm are considered to be equally suitable for use in the invention. CDRs may vary in length, depending on the antibody from which they are predicted and between the heavy and light chains. Thus, the three heavy chain CDRs of an intact antibody may be of different lengths (or may be of the same length) and the three light chain CDRs of an intact antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids.

Suitable anti-TNF antibodies are known in the art. Moreover, anti-TNF antibodies, and fragments and/or derivatives thereof, can be prepared using methods known by those of skill in the art. Such methods include phage display, methods to generate human or humanized antibodies, or methods using transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence encoding for the antibody (or fragment and/or derivative thereof) can be isolated and/or determined. The sequence of the antibody can be used to design suitable fragments and/or derivatives thereof.

In preferred embodiments, the anti-TNF antibody or fragment thereof is an anti-TNF antibody fragment. An “anti-TNF antibody fragment” may be a fragment of an anti-TNF antibody, or a genetically engineered product of one of more fragments of the anti-TNF antibody, which fragment is involved in binding with TNF. Examples include an antigen-binding fragment (Fab), a fragment antibody (F(ab′)₂), a variable region (Fv), a single chain antibody (scFv), a single-domain antibody (sdAb), and a camelid antibody (VHH). The use of an anti-TNF antibody fragment may be advantageous because it may reduce inflammation associated with the Fc region.

In some embodiments, the anti-TNF antibody fragment is an antigen-binding fragment (Fab), a fragment antibody (F(ab′)₂), a single chain antibody (scFv), or a single-domain antibody (sdAb).

In preferred embodiments, the anti-TNF antibody fragment is an antigen-binding fragment (Fab). “Antigen-binding fragment” (Fab) refers to a region on an antibody that binds to antigens. It is composed of one constant and one variable region of each of the heavy and the light chain.

In other embodiments, the anti-TNF antibody fragment is a fragment antibody (F(ab′)₂). “Fragment antibody” (F(ab′)₂) refers to a region on an antibody that remains following digestion of the Fc region while leaving intact some of the hinge region.

In other embodiments, the anti-TNF antibody fragment is a single chain antibody (scFv). “Single chain antibody” (scFv) refers to an engineered antibody consisting of a light chain variable region and a heavy chain variable region connected to one another directly or via a peptide linker sequence. The peptide linker sequence is usually about 10 to 25 amino acids in length, rich in glycine for flexibility, and serine or threonine for solubility. The peptide linker sequence can either connect the N-terminus of the heavy chain variable region with the C-terminus of the light chain variable region, or vice versa.

In other embodiments, the anti-TNF antibody fragment is a single-domain antibody (sdAb). “Single-domain antibody” (sdAb), also known as a nanobody, refers to an antibody fragment consisting of a single monomeric variable antibody domain. Accordingly, a sdAb may be a heavy chain variable region (VH) or a light chain variable region (VL). Examples of single-domain antibodies include, but are not limited to, VHH fragments, and VNAR fragments. Single-domain antibodies may also be generated by splitting the dimeric variable domains from common IgG molecules into monomers.

The TNF inhibitor may comprise at least one CDR (e.g. HCDR3), which can be predicted from an anti-TNF antibody (or a variant of such a predicted CDR e.g. a variant with one, two or three amino acid substitutions). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (see e.g. Vaughan and Sollazzo, 2001, Combinational Chemistry & High Throughput Screening, 4, 417-430). Molecules containing a single CDR have been described which can display strong binding activity to target (see e.g. Nicaise et al, 2004, Protein Science, 13:1882-91).

The TNF inhibitor may comprise one or more variable heavy chain CDRs, e.g. one, two or three variable heavy chain CDRs. Alternatively, or additionally, the TNF inhibitor may comprise one or more variable light chain CDRs, e.g. one, two or three variable light chain CDRs. The TNF inhibitor may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an anti-TNF antibody, or may be selected from one of the CDR sequences provided below.

The TNF inhibitor may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the TNF inhibitor comprises three variable heavy chain CDRs (HCDR1, HCDR2 and HCDR3) and/or three variable light chain CDRs (LCDR1, LCDR2 and LCDR3).

The one or more CDRs present within the TNF inhibitor may not all be from the same antibody, as long as the domain has the binding activity described above. Thus, one CDR may be predicted from the heavy or light chains of an anti-TNF antibody, whilst another CDR present may be predicted from a different anti-TNF antibody. In this instance, it may be preferred that CDR3 be predicted from an anti-TNF antibody. Particularly however, if more than one CDR is present in the TNF inhibitor, it is preferred that the CDRs are predicted from anti-TNF antibodies. A combination of CDRs may be used from different antibodies, particularly from antibodies that bind to the same desired region or epitope.

In a preferred embodiment, the TNF inhibitor comprises three CDRs predicted from the variable heavy chain sequence of an anti-TNF antibody and/or three CDRs predicted from the variable light chain sequence of an anti-TNF antibody.

The present invention includes “variants” of the CDR regions described below. The term “variant” as used herein is defined below in the section “Variants, derivatives and fragments”. It will be appreciated that one or more amino acid substitutions may be made in the CDRs whilst retaining the antigen-binding ability. For instance, the CDR variants may comprise 3 or fewer amino acid substitutions, e.g. 3 amino acid substitutions, 2 amino acid substitutions or 1 amino acid substitution. In particular, in some embodiments the CDR variants comprise one amino acid substitution and retain the antigen-binding ability. The variant may be a variant with at least 80% or 90% identity to the CDR.

Examples of antibodies, and fragments and/or derivatives thereof that can be used in the invention are further described below. The TNF inhibitor may comprise or consist of an amino acid sequence comprising the CDRs described herein and substitutions, variations, modifications, replacements, deletions and/or additions of one or more amino acid residues may occur in the framework region. The derivatives described herein may retain TNF-binding ability. The derivatives may be capable of binding TNF to at least 10%, at least 20%, at least 30%, at least 40%, at least 50%, at least 60%, at least 70%, at least 80%, at least 90%, or 100% of the level of the corresponding reference amino acid sequence.

TNF-binding affinity may be determined by equilibrium binding constants (KD), which may be determined by any suitable assay e.g. surface plasmon resonance (see e.g. Shealy, D. J., et al., 2010. mAbs, 2 (4), pp. 428-439). Suitably, an anti-TNF antibody or a fragment thereof may bind to soluble TNF with a binding affinity of about 1000 pM or less, about 900 pM or less, about 800 pM or less, about 700 pM or less, about 600 pM or less, about 500 pM or less, about 400 pM or less, about 300 pM or less, about 200 pM or less, or about 100 pM or less, for example as determined by surface plasmon resonance. Suitably, an anti-TNF antibody or a fragment thereof may bind to soluble TNF with a binding affinity of from about 0.1 pM to about 1000 pM, from about 1 pM to about 1000 pM, from about 10 pM to about 1000 pM, 0.1 pM to about 100 pM, from about 1 pM to about 100 pM, or from about 10 pM to about 100 pM, for example as determined by surface plasmon resonance. Suitably, an anti-TNF antibody or a fragment thereof may bind to transmembrane TNF with a binding affinity of about 10000 pM or less, about 9000 pM or less, about 8000 pM or less, about 7000 pM or less, about 6000 pM or less, about 5000 pM or less, or about 4000 pM or less, for example as determined by surface plasmon resonance.

In some embodiments, the TNF inhibitor is selected from any of: adalimumab, or a fragment and/or derivative thereof; infliximab, or a fragment and/or derivative thereof; golimumab, or a fragment and/or derivative thereof; and certolizumab or a fragment and/or derivative thereof.

In some embodiments, the TNF inhibitor is selected from: adalimumab, or a fragment and/or derivative thereof; and infliximab, or a fragment and/or derivative thereof.

In some embodiments, the TNF inhibitor is selected from any of: an adalimumab fragment or derivative thereof; an infliximab fragment or derivative thereof; a golimumab fragment or derivative thereof; and a certolizumab fragment or derivative thereof.

In some embodiments, the TNF inhibitor is selected from any of: an adalimumab fragment or derivative thereof; and an infliximab fragment or derivative thereof.

### Adalimumab

In preferred embodiments, the TNF inhibitor is adalimumab, or a fragment and/or derivative thereof. In some embodiments, the TNF inhibitor is an adalimumab Fab, an adalimumab F(ab′)₂, an adalimumab scFv, an adalimumab sdAb, or a derivative thereof. In some embodiments, the TNF inhibitor is an adalimumab Fab, or a derivative thereof.

Adalimumab (Humira®) is a recombinant, fully human IgG1 monoclonal antibody that binds specifically to TNF, thereby neutralizing the activity of the cytokine. The skilled person would be able to generate adalimumab derivatives using conservative mutations and/or knowledge of the mechanism of adalimumab inhibition of TNF (see e.g. Hu, S., et al., 2013. Journal of Biological Chemistry, 288 (38), pp. 27059-27067). For example, the following adalimumab variants have been shown to have comparable KD values to wild-type adalimumab: L178K, L178N, Q160N, L116N, T118N, A122N, Q179N, L183N, and T199N (see e.g. Reslan, M., et al., 2020. International Journal of Biological Macromolecules, 158, pp. 189-196).

In some embodiments, the TNF inhibitor is a fragment of adalimumab, or a derivative thereof. In some embodiments, the TNF inhibitor is an adalimumab Fab, an adalimumab F(ab′)₂, an adalimumab scFv, or an adalimumab sdAb, or a derivative thereof. In some embodiments, the TNF inhibitor is an adalimumab Fab, or a derivative thereof.

Suitable adalimumab Fab derivatives include those described in Yoshikawa, M., et al., 2022. The Journal of Biochemistry, mvac040; and Nakamura, H., et al., 2020. Biological and Pharmaceutical Bulletin, 43 (3), pp. 418-423. Suitably, a adalimumab Fab derivative may be selected from one or more of: H:K137C-L:I117C, H:K137C-L:F209C, H:S138C-L:F116C, H:S140C-L:S114C, and H:V177C-L:Q160C.

In some embodiments, the TNF inhibitor is, or is derived from, an anti-TNF antibody (e.g. is a Fab, F(ab′)₂, scFv, or sdAb) wherein the antibody comprises one or more CDR regions, selected from SEQ ID NOs: 1-6, or variants thereof. In other words, in some embodiments the TNF inhibitor comprises one or more CDR regions, selected from SEQ ID NOs: 1-6, or variants thereof.

In some embodiments, the TNF inhibitor comprises:
(i) a HCDR1 having an amino acid sequence of SEQ ID NO: 1 or a variant thereof, a HCDR2 having an amino acid sequence of SEQ ID NO: 2 or a variant thereof, and/or a HCDR3 having an amino acid sequence of SEQ ID NO: 3 or a variant thereof; and/or
(ii) a LCDR1 having an amino acid sequence of SEQ ID NO: 4 or a variant thereof, a LCDR2 having an amino acid sequence of SEQ ID NO: 5 or a variant thereof, and/or a LCDR3 having an amino acid sequence of SEQ ID NO: 6 or a variant thereof.

In some embodiments, the TNF inhibitor comprises a HCDR2 having an amino acid sequence of SEQ ID NO: 2 or a variant thereof and/or a LCDR2 having an amino acid sequence of SEQ ID NO: 5 or a variant thereof. For adalimumab, CDRs L2 and H2 contribute to the majority of the interactions with the antigen (see Hu, S., et al., 2013. Journal of Biological Chemistry, 288 (38), pp. 27059-27067).

In some embodiments, the TNF inhibitor comprises:
(i) a HCDR1 having an amino acid sequence of SEQ ID NO: 1 or a variant thereof, a HCDR2 having an amino acid sequence of SEQ ID NO: 2 or a variant thereof, and a HCDR3 having an amino acid sequence of SEQ ID NO: 3 or a variant thereof; and/or
(ii) a LCDR1 having an amino acid sequence of SEQ ID NO: 4 or a variant thereof, a LCDR2 having an amino acid sequence of SEQ ID NO: 5 or a variant thereof, and a LCDR3 having an amino acid sequence of SEQ ID NO: 6 or a variant thereof.

In some embodiments, the TNF inhibitor comprises or consists of a heavy chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 7.

In some embodiments, the TNF inhibitor comprises or consists of a light chain, wherein the light chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 8.

In some embodiments, the TNF inhibitor comprises or consists of a heavy chain and a light chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 7, and the light chain comprises an amino acid sequence having at least 70%, at least 75%, at least 80%, at least 85%, at least 90%, at least 95%, at least 96%, at least 97%, at least 98%, at least 99% identity or 100% identity to SEQ ID NO: 8.

In some embodiments, the TNF inhibitor comprises or consists of:
(i) a heavy chain comprising an amino acid sequence having at least 70%, 75%, 80%, 85%, 90%, 95%, 96%, 97%, 98%, 99%, or 100% identity to SEQ ID NO: 7, wherein the amino acid sequence comprises a HCDR1 having an amino acid sequence of SEQ ID NO: 1 or a variant thereof, a HCDR2 having an amino acid sequence of SEQ ID NO: 2 or a variant thereof, and a HCDR3 having an amino acid sequence of SEQ ID NO: 3 or a variant thereof; and/or
(ii) a light chain comprising an amino acid sequence having at least 70%, 75%, 80%, 85%, 90%, 95%, 96%, 97%, 98%, 99%, or 100% identity to SEQ ID NO: 8, wherein the amino acid sequence comprises a LCDR1 having an amino acid sequence of SEQ ID NO: 4 or a variant thereof, a LCDR2 having an amino acid sequence of SEQ ID NO: 5 or a variant thereof, and a LCDR3 having an amino acid sequence of SEQ ID NO: 6 or a variant thereof.

In some embodiments, the TNF inhibitor comprises or consists of a heavy chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 9 or 10.

In some embodiments, the TNF inhibitor comprises or consists of a light chain, wherein the light chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 11.

In some embodiments, the TNF inhibitor comprises or consists of a heavy chain and a light chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 9, and the light chain comprises or consists of an amino acid sequence having at least 70%, at least 75%, at least 80%, at least 85%, at least 90%, at least 95%, at least 96%, at least 97%, at least 98%, at least 99% identity or 100% identity to SEQ ID NO: 11.

In some embodiments, the TNF inhibitor comprises or consists of:
(i) a heavy chain comprising or consisting of an amino acid sequence having at least 70%, 75%, 80%, 85%, 90%, 95%, 96%, 97%, 98%, 99%, or 100% identity to SEQ ID NO: 9, wherein the amino acid sequence comprises a HCDR1 having an amino acid sequence of SEQ ID NO: 1, a HCDR2 having an amino acid sequence of SEQ ID NO: 2, and a HCDR3 having an amino acid sequence of SEQ ID NO: 3; and/or
(ii) a light chain comprising or consisting of an amino acid sequence having at least 70%, 75%, 80%, 85%, 90%, 95%, 96%, 97%, 98%, 99%, or 100% identity to SEQ ID NO: 11, wherein the amino acid sequence comprises a LCDR1 having an amino acid sequence of SEQ ID NO: 4, a LCDR2 having an amino acid sequence of SEQ ID NO: 5, and a LCDR3 having an amino acid sequence of SEQ ID NO: 6.

| Name | Example sequence |
|---|---|
| Adalimumab HCDR1 | TFDDYA (SEQ ID NO: 1) |
| Adalimumab HCDR2 | TWNSGHID (SEQ ID NO: 2) |
| Adalimumab HCDR3 | VSYLSTASSL (SEQ ID NO: 3) |
| Adalimumab LCDR1 | GIRNYLA (SEQ ID NO: 4) |
| Adalimumab LCDR2 | YAASTLQ (SEQ ID NO: 5) |
| Adalimumab LCDR3 | RYNRA (SEQ ID NO: 6) |
| Adalimumab VH | EVQLVESGGGLVQPGRSLRLSCAASGFTFDDYAMHWVRQAPGKGLEWVSA |
| | ITWNSGHIDYADSVEGRFTISRDNAKNSLYLQMNSLRAEDTAVYYCAKVS |
| | YLSTASSLDYWGQGTLVTVS (SEQ ID NO: 7) |
| Adalimumab VL | DIQMTQSPSSLSASVGDRVTITCRASQGIRNYLAWYQQKPGKAPKLLIYA |
| | ASTLQSGVPSRFSGSGSGTDFTLTISSLQPEDVATYYCQRYNRAPYTFGQ |
| | GTKVEIK (SEQ ID NO: 8) |
| Adalimumab VH + | EVQLVESGGGLVQPGRSLRLSCAASGFTFDDYAMHWVRQAPGKGLEWVSA |
| CH1 | ITWNSGHIDYADSVEGRFTISRDNAKNSLYLQMNSLRAEDTAVYYCAKVS |
| | YLSTASSLDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQ |
| | TYICNVNHKPSNTKVDKKV (SEQ ID NO: 9) |
| Adalimumab heavy | EVQLVESGGGLVQPGRSLRLSCAASGFTFDDYAMHWVRQAPGKGLEWVSA |
| chain | ITWNSGHIDYADSVEGRFTISRDNAKNSLYLQMNSLRAEDTAVYYCAKVS |
| | YLSTASSLDYWGQGTLVTVSSASTKGPSVFPLAPSSKSTSGGTAALGCLV |
| | KDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPK |
| | PKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPG |
| | K (SEQ ID NO: 10) |
| Adalimumab light | DIQMTQSPSSLSASVGDRVTITCRASQGIRNYLAWYQQKPGKAPKLLIYA |
| chain | ASTLQSGVPSRFSGSGSGTDFTLTISSLQPEDVATYYCQRYNRAPYTFGQ |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC (SEQ ID NO: 11) |

### Infliximab

In some embodiments, the TNF inhibitor is infliximab, or a fragment and/or derivative thereof. In some embodiments, the TNF inhibitor is an infliximab Fab, an infliximab F(ab′)₂, an infliximab scFv, an infliximab sdAb, or a derivative thereof. In some embodiments, the TNF inhibitor is an infliximab Fab, or a derivative thereof.

Infliximab (Remicade®) is a chimeric monoclonal antibody to human TNF. It binds to both soluble and transmembrane forms of TNF at picomolar concentrations. The skilled person would be able to generate infliximab derivatives using conservative mutations and/or knowledge of the mechanism of infliximab inhibition of TNF (see e.g. Liang, S., et al., 2013. Journal of Biological Chemistry, 288 (19), pp. 13799-13807).

In some embodiments, the TNF inhibitor is a fragment of infliximab, or a derivative thereof. In some embodiments, the TNF inhibitor is an infliximab Fab, an infliximab F(ab′)₂, an infliximab scFv, or an infliximab sdAb, or a derivative thereof. In some embodiments, the TNF inhibitor is an infliximab Fab, or a derivative thereof.

In some embodiments, the TNF inhibitor is, or is derived from, an anti-TNF antibody (e.g. is a Fab, F(ab′)₂, scFv, or sdAb) wherein the antibody comprises one or more CDR regions, selected from SEQ ID NOs: 12-17, or variants thereof. In other words, in some embodiments the TNF inhibitor comprises one or more CDR regions, selected from SEQ ID NOs: 12-17, or variants thereof.

In some embodiments, the TNF inhibitor comprises:
(i) a HCDR1 having an amino acid sequence of SEQ ID NO: 12 or a variant thereof, a HCDR2 having an amino acid sequence of SEQ ID NO: 13 or a variant thereof, and/or a HCDR3 having an amino acid sequence of SEQ ID NO: 14 or a variant thereof; and/or
(ii) a LCDR1 having an amino acid sequence of SEQ ID NO: 15 or a variant thereof, a LCDR2 having an amino acid sequence of SEQ ID NO: 16 or a variant thereof, and/or a LCDR3 having an amino acid sequence of SEQ ID NO: 17 or a variant thereof.

In some embodiments, the TNF inhibitor comprises:
(i) a HCDR1 having an amino acid sequence of SEQ ID NO: 12 or a variant thereof, a HCDR2 having an amino acid sequence of SEQ ID NO: 13 or a variant thereof, and a HCDR3 having an amino acid sequence of SEQ ID NO: 14 or a variant thereof; and/or
(ii) a LCDR1 having an amino acid sequence of SEQ ID NO: 15 or a variant thereof, a LCDR2 having an amino acid sequence of SEQ ID NO: 16 or a variant thereof, and a LCDR3 having an amino acid sequence of SEQ ID NO: 17 or a variant thereof.

In some embodiments, the TNF inhibitor comprises or consists of a heavy chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 18.

In some embodiments, the TNF inhibitor comprises or consists of a light chain, wherein the light chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 19.

In some embodiments, the TNF inhibitor comprises or consists of a heavy chain and a light chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 18, and the light chain comprises an amino acid sequence having at least 70%, at least 75%, at least 80%, at least 85%, at least 90%, at least 95%, at least 96%, at least 97%, at least 98%, at least 99% identity or 100% identity to SEQ ID NO: 19.

In some embodiments, the TNF inhibitor comprises or consists of:
(i) a heavy chain comprising an amino acid sequence having at least 70%, 75%, 80%, 85%, 90%, 95%, 96%, 97%, 98%, 99%, or 100% identity to SEQ ID NO: 18, wherein the amino acid sequence comprises a HCDR1 having an amino acid sequence of SEQ ID NO: 12 or a variant thereof, a HCDR2 having an amino acid sequence of SEQ ID NO: 13 or a variant thereof, and a HCDR3 having an amino acid sequence of SEQ ID NO: 14 or a variant thereof; and/or
(ii) a light chain comprising an amino acid sequence having at least 70%, 75%, 80%, 85%, 90%, 95%, 96%, 97%, 98%, 99%, or 100% identity to SEQ ID NO: 19, wherein the amino acid sequence comprises a LCDR1 having an amino acid sequence of SEQ ID NO: 15 or a variant thereof, a LCDR2 having an amino acid sequence of SEQ ID NO: 16 or a variant thereof, and a LCDR3 having an amino acid sequence of SEQ ID NO: 17 or a variant thereof.

In some embodiments, the TNF inhibitor comprises or consists of a heavy chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 20 or 21.

In some embodiments, the TNF inhibitor comprises or consists of a light chain, wherein the light chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 22.

In some embodiments, the TNF inhibitor comprises or consists of a heavy chain and a light chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 20, and the light chain comprises or consists of an amino acid sequence having at least 70%, at least 75%, at least 80%, at least 85%, at least 90%, at least 95%, at least 96%, at least 97%, at least 98%, at least 99% identity or 100% identity to SEQ ID NO: 22.

In some embodiments, the TNF inhibitor comprises or consists of:
(i) a heavy chain comprising or consisting of an amino acid sequence having at least 70%, 75%, 80%, 85%, 90%, 95%, 96%, 97%, 98%, 99%, or 100% identity to SEQ ID NO: 20, wherein the amino acid sequence comprises a HCDR1 having an amino acid sequence of SEQ ID NO: 12, a HCDR2 having an amino acid sequence of SEQ ID NO: 13, and a HCDR3 having an amino acid sequence of SEQ ID NO: 14; and/or
(ii) a light chain comprising or consisting of an amino acid sequence having at least 70%, 75%, 80%, 85%, 90%, 95%, 96%, 97%, 98%, 99%, or 100% identity to SEQ ID NO: 22, wherein the amino acid sequence comprises a LCDR1 having an amino acid sequence of SEQ ID NO: 15, a LCDR2 having an amino acid sequence of SEQ ID NO: 16, and a LCDR3 having an amino acid sequence of SEQ ID NO: 17.

| Name | Example sequence |
|---|---|
| Infliximab HCDR1 | IFSNHW (SEQ ID NO: 12) |
| Infliximab HCDR2 | RSKSINSATH (SEQ ID NO: 13) |
| Infliximab HCDR3 | NYYGSTY (SEQ ID NO: 14) |
| Infliximab LCDR1 | FVGSSIH (SEQ ID NO: 15) |
| Infliximab LCDR2 | KYASESM (SEQ ID NO: 16) |
| Infliximab LCDR3 | QSHSW (SEQ ID NO: 17) |
| Infliximab VH | EVKLEESGGGLVQPGGSMKLSCVASGFIFSNHWMNWVRQSPEKGLEWVAE |
| | IRSKSINSATHYAESVKGRFTISRDDSKSAVYLQMTDLRTEDTGVYYCSR |
| | NYYGSTYDYWGQGTTLTVSS (SEQ ID NO: 18) |
| Infliximab VL | DILLTQSPAILSVSPGERVSFSCRASQFVGSSIHWYQQRTNGSPRLLIKY |
| | ASESMSGIPSRFSGSGSGTDFTLSINTVESEDIADYYCQQSHSWPFTFGS |
| | GTNLEVK (SEQ ID NO: 19) |
| Infliximab VH + | EVKLEESGGGLVQPGGSMKLSCVASGFIFSNHWMNWVRQSPEKGLEWVAE |
| CH1 | IRSKSINSATHYAESVKGRFTISRDDSKSAVYLQMTDLRTEDTGVYYCSR |
| | NYYGSTYDYWGQGTTLTVSSASTKGPSVFPLAPSSKSTSGGTAALGCLVK |
| | DYFPEPVTVSWNSGALTSGVHTFPAVLQSSGLYSLSSVVTVPSSSLGTQT |
| | YICNVNHKPSNTKVDKKV (SEQ ID NO: 20) |
| Infliximab heavy | EVKLEESGGGLVQPGGSMKLSCVASGFIFSNHWMNWVRQSPEKGLEWVAE |
| chain | IRSKSINSATHYAESVKGRFTISRDDSKSAVYLQMTDLRTEDTGVYYCSR |
| | NYYGSTYDYWGQGTTLTVSSASTKGPSVFPLAPSSKSTSGGTAALGCLVK |
| | DYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKP |
| | KDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | (SEQ ID NO: 21) |
| Infliximab light | DILLTQSPAILSVSPGERVSFSCRASQFVGSSIHWYQQRTNGSPRLLIKY |
| chain | ASESMSGIPSRFSGSGSGTDFTLSINTVESEDIADYYCQQSHSWPFTFGS |
| | GTNLEVKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC (SEQ ID NO: 22) |

### Golimumab

In some embodiments, the TNF inhibitor is golimumab, or a fragment and/or derivative thereof. In some embodiments, the TNF inhibitor is a golimumab Fab, a golimumab F(ab′)₂, a golimumab scFv, a golimumab sdAb, or a derivative thereof. In some embodiments, the TNF inhibitor is a golimumab Fab, or a derivative thereof.

Golimumab (Simponi®) is a human IgG1 TNF antagonist monoclonal antibody. The skilled person would be able to generate golimumab derivatives using conservative mutations and/or knowledge of the mechanism of golimumab inhibition of TNF (see e.g. Shealy, D. J., et al., 2010. MAbs, 2 (4), pp. 428-439).

In some embodiments, the TNF inhibitor is a fragment of golimumab, or a derivative thereof. In some embodiments, the TNF inhibitor is a golimumab Fab, a golimumab F(ab′)₂, a golimumab scFv, or a golimumab sdAb, or a derivative thereof. In some embodiments, the TNF inhibitor is a golimumab Fab, or a derivative thereof.

In some embodiments, the TNF inhibitor is, or is derived from an anti-TNF antibody (e.g. is a Fab, F(ab′)₂, scFv, or sdAb) wherein the antibody comprises one or more CDR regions, selected from SEQ ID NOs: 23-28, or variants thereof. In other words, in some embodiments the TNF inhibitor comprises one or more CDR regions, selected from SEQ ID NOs: 23-28, or variants thereof.

In some embodiments, the TNF inhibitor comprises:
(i) a HCDR1 having an amino acid sequence of SEQ ID NO: 23 or a variant thereof, a HCDR2 having an amino acid sequence of SEQ ID NO: 24 or a variant thereof, and/or a HCDR3 having an amino acid sequence of SEQ ID NO: 25 or a variant thereof; and/or
(ii) a LCDR1 having an amino acid sequence of SEQ ID NO: 26 or a variant thereof, a LCDR2 having an amino acid sequence of SEQ ID NO: 27 or a variant thereof, and/or a LCDR3 having an amino acid sequence of SEQ ID NO: 28 or a variant thereof.

In some embodiments, the TNF inhibitor comprises:
(i) a HCDR1 having an amino acid sequence of SEQ ID NO: 23 or a variant thereof, a HCDR2 having an amino acid sequence of SEQ ID NO: 24 or a variant thereof, and a HCDR3 having an amino acid sequence of SEQ ID NO: 25 or a variant thereof; and/or
(ii) a LCDR1 having an amino acid sequence of SEQ ID NO: 26 or a variant thereof, a LCDR2 having an amino acid sequence of SEQ ID NO: 27 or a variant thereof, and a LCDR3 having an amino acid sequence of SEQ ID NO: 28 or a variant thereof.

In some embodiments, the TNF inhibitor comprises or consists of a heavy chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 29.

In some embodiments, the TNF inhibitor comprises or consists of a light chain, wherein the light chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 30.

In some embodiments, the TNF inhibitor comprises or consists of a heavy chain and a light chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 29, and the light chain comprises an amino acid sequence having at least 70%, at least 75%, at least 80%, at least 85%, at least 90%, at least 95%, at least 96%, at least 97%, at least 98%, at least 99% identity or 100% identity to SEQ ID NO: 30.

In some embodiments, the TNF inhibitor comprises or consists of:
(i) a heavy chain comprising an amino acid sequence having at least 70%, 75%, 80%, 85%, 90%, 95%, 96%, 97%, 98%, 99%, or 100% identity to SEQ ID NO: 29, wherein the amino acid sequence comprises a HCDR1 having an amino acid sequence of SEQ ID NO: 23 or a variant thereof, a HCDR2 having an amino acid sequence of SEQ ID NO: 24 or a variant thereof, and a HCDR3 having an amino acid sequence of SEQ ID NO: 25 or a variant thereof; and/or
(ii) a light chain comprising an amino acid sequence having at least 70%, 75%, 80%, 85%, 90%, 95%, 96%, 97%, 98%, 99%, or 100% identity to SEQ ID NO: 30, wherein the amino acid sequence comprises a LCDR1 having an amino acid sequence of SEQ ID NO: 26 or a variant thereof, a LCDR2 having an amino acid sequence of SEQ ID NO: 27 or a variant thereof, and a LCDR3 having an amino acid sequence of SEQ ID NO: 28 or a variant thereof.

In some embodiments, the TNF inhibitor comprises or consists of a heavy chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 31 or 32.

In some embodiments, the TNF inhibitor comprises or consists of a light chain, wherein the light chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 33.

In some embodiments, the TNF inhibitor comprises or consists of a heavy chain and a light chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 31, and the light chain comprises or consists of an amino acid sequence having at least 70%, at least 75%, at least 80%, at least 85%, at least 90%, at least 95%, at least 96%, at least 97%, at least 98%, at least 99% identity or 100% identity to SEQ ID NO: 33.

In some embodiments, the TNF inhibitor comprises or consists of:
(i) a heavy chain comprising or consisting of an amino acid sequence having at least 70%, 75%, 80%, 85%, 90%, 95%, 96%, 97%, 98%, 99%, or 100% identity to SEQ ID NO: 31, wherein the amino acid sequence comprises a HCDR1 having an amino acid sequence of SEQ ID NO: 23, a HCDR2 having an amino acid sequence of SEQ ID NO: 24, and a HCDR3 having an amino acid sequence of SEQ ID NO: 25; and/or
(ii) a light chain comprising or consisting of an amino acid sequence having at least 70%, 75%, 80%, 85%, 90%, 95%, 96%, 97%, 98%, 99%, or 100% identity to SEQ ID NO: 33, wherein the amino acid sequence comprises a LCDR1 having an amino acid sequence of SEQ ID NO: 26, a LCDR2 having an amino acid sequence of SEQ ID NO: 27, and a LCDR3 having an amino acid sequence of SEQ ID NO: 28.

| Name | Example sequence |
|---|---|
| Golimumab HCDR1 | IFSSYA (SEQ ID NO: 23) |
| Golimumab HCDR2 | SYDGSNKK (SEQ ID NO: 24) |
| Golimumab HCDR3 | NYYYYGM (SEQ ID NO: 25) |
| Golimumab LCDR1 | SVYSYLA (SEQ ID NO: 26) |
| Golimumab LCDR2 | YDASNRA (SEQ ID NO: 27) |
| Golimumab LCDR3 | QRSNW (SEQ ID NO: 28) |
| Golimumab VH | QVQLVESGGGVVQPGRSLRLSCAASGFIFSSYAMHWVRQAPGNGLEWVAF |
| | MSYDGSNKKYADSVKGRFTISRDNSKNTLYLQMNSLRAEDTAVYYCARDR |
| | GIAAGGNYYYYGMDVWGQGTTVTVSS (SEQ ID NO: 29) |
| Golimumab VL | EIVLTQSPATLSLSPGERATLSCRASQSVYSYLAWYQQKPGQAPRLLIYD |
| | ASNRATGIPARFSGSGSGTDFTLTISSLEPEDFAVYYCQQRSNWPPFTFG |
| | PGTKVDIK (SEQ ID NO: 30) |
| Golimumab VH + | QVQLVESGGGVVQPGRSLRLSCAASGFIFSSYAMHWVRQAPGNGLEWVAF |
| CH1 | MSYDGSNKKYADSVKGRFTISRDNSKNTLYLQMNSLRAEDTAVYYCARDR |
| | GIAAGGNYYYYGMDVWGQGTTVTVSSASTKGPSVFPLAPSSKSTSGGTAA |
| | LGCLVKDYFPEPVTVSWNSGALTSGVHTFPAVLQSSGLYSLSSVVTVPSS |
| | SLGTQTYICNVNHKPSNTKVDKKV (SEQ ID NO: 31) |
| Golimumab heavy | QVQLVESGGGVVQPGRSLRLSCAASGFIFSSYAMHWVRQAPGNGLEWVAF |
| chain | MSYDGSNKKYADSVKGRFTISRDNSKNTLYLQMNSLRAEDTAVYYCARDR |
| | GIAAGGNYYYYGMDVWGQGTTVTVSSASTKGPSVFPLAPSSKSTSGGTAA |
| | LGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPSVF |
| | LFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSL |
| | SLSPGK (SEQ ID NO: 32) |
| Golimumab light | EIVLTQSPATLSLSPGERATLSCRASQSVYSYLAWYQQKPGQAPRLLIYD |
| chain | ASNRATGIPARFSGSGSGTDFTLTISSLEPEDFAVYYCQQRSNWPPFTFG |
| | PGTKVDIKRTVAAPSVFIFPPSDEQLKSGTASVVCLLLLECINENNFYPR |
| | EAKVQWKVDNALQSGNSQESVTEQDSKDSTYSLSSTLTLSKADYEKHKVY |
| | ACEVTHQGLSSPVTKSFNRGEC (SEQ ID NO: 33) |

### Certolizumab

In some embodiments, the TNF inhibitor is certolizumab, or a fragment and/or derivative thereof. In some embodiments, the TNF inhibitor is a certolizumab Fab, a certolizumab F(ab′)₂, a certolizumab scFv, a certolizumab sdAb, or a derivative thereof. In some embodiments, the TNF inhibitor is a certolizumab Fab, or a derivative thereof.

Certolizumab is a humanized antigen-binding fragment (Fab′) of a monoclonal antibody, that is usually administered in a form that is conjugated to polyethylene glycol (Cimzia®). The skilled person would be able to generate certolizumab derivatives using conservative mutations and/or knowledge of the mechanism of certolizumab inhibition of TNF (see e.g. Lee, J. U., et al., 2017. International journal of molecular sciences, 18 (1), p. 228).

In some embodiments, the TNF inhibitor is a fragment of certolizumab, or a derivative thereof. In some embodiments, the TNF inhibitor is a certolizumab Fab, a certolizumab F(ab′)₂, a certolizumab scFv, or a certolizumab sdAb, or a derivative thereof. In some embodiments, the TNF inhibitor is a certolizumab Fab, or a derivative thereof.

In some embodiments, the TNF inhibitor is, or is derived from an anti-TNF antibody (e.g. is a Fab, F(ab′)₂, scFv, or sdAb) wherein the antibody comprises one or more CDR regions, selected from SEQ ID NOs: 34-39, or variants thereof. In other words, in some embodiments the TNF inhibitor comprises one or more CDR regions, selected from SEQ ID NOs: 34-39, or variants thereof.

In some embodiments, the TNF inhibitor comprises:
(i) a HCDR1 having an amino acid sequence of SEQ ID NO: 34 or a variant thereof, a HCDR2 having an amino acid sequence of SEQ ID NO: 35 or a variant thereof, and/or a HCDR3 having an amino acid sequence of SEQ ID NO: 36 or a variant thereof; and/or
(ii) a LCDR1 having an amino acid sequence of SEQ ID NO: 37 or a variant thereof, a LCDR2 having an amino acid sequence of SEQ ID NO: 38 or a variant thereof, and/or a LCDR3 having an amino acid sequence of SEQ ID NO: 39 or a variant thereof.

In some embodiments, the TNF inhibitor comprises:
(i) a HCDR1 having an amino acid sequence of SEQ ID NO: 34 or a variant thereof, a HCDR2 having an amino acid sequence of SEQ ID NO: 35 or a variant thereof, and a HCDR3 having an amino acid sequence of SEQ ID NO: 36 or a variant thereof; and/or
(ii) a LCDR1 having an amino acid sequence of SEQ ID NO: 37 or a variant thereof, a LCDR2 having an amino acid sequence of SEQ ID NO: 38 or a variant thereof, and a LCDR3 having an amino acid sequence of SEQ ID NO: 39 or a variant thereof.

In some embodiments, the TNF inhibitor comprises or consists of a heavy chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 40.

In some embodiments, the TNF inhibitor comprises or consists of a light chain, wherein the light chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 41.

In some embodiments, the TNF inhibitor comprises or consists of a heavy chain and a light chain, wherein the heavy chain comprises an amino acid sequence having at least 70%, at least 75%, at least 80%, at least 85%, at least 90%, at least 95%, at least 96%, at least 97%, at least 98%, at least 99% identity, or 100% identity to SEQ ID NO: 40, and the light chain comprises an amino acid sequence having at least 70%, at least 75%, at least 80%, at least 85%, at least 90%, at least 95%, at least 96%, at least 97%, at least 98%, at least 99% identity or 100% identity to SEQ ID NO: 41.

In some embodiments, the TNF inhibitor comprises or consists of:
(i) a heavy chain comprising an amino acid sequence having at least 70%, 75%, 80%, 85%, 90%, 95%, 96%, 97%, 98%, 99%, or 100% identity to SEQ ID NO: 40, wherein the amino acid sequence comprises a HCDR1 having an amino acid sequence of SEQ ID NO: 34 or a variant thereof, a HCDR2 having an amino acid sequence of SEQ ID NO: 35 or a variant thereof, and a HCDR3 having an amino acid sequence of SEQ ID NO: 36 or a variant thereof; and/or
(ii) a light chain comprising an amino acid sequence having at least 70%, 75%, 80%, 85%, 90%, 95%, 96%, 97%, 98%, 99%, or 100% identity to SEQ ID NO: 41, wherein the amino acid sequence comprises a LCDR1 having an amino acid sequence of SEQ ID NO: 37 or a variant thereof, a LCDR2 having an amino acid sequence of SEQ ID NO: 38 or a variant thereof, and a LCDR3 having an amino acid sequence of SEQ ID NO: 39 or a variant thereof.

In some embodiments, the TNF inhibitor comprises or consists of a heavy chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 42 or 43.

In some embodiments, the TNF inhibitor comprises or consists of a light chain, wherein the light chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 44.

In some embodiments, the TNF inhibitor comprises or consists of a heavy chain and a light chain, wherein the heavy chain comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 42, and the light chain comprises or consists of an amino acid sequence having at least 70%, at least 75%, at least 80%, at least 85%, at least 90%, at least 95%, at least 96%, at least 97%, at least 98%, at least 99% identity or 100% identity to SEQ ID NO: 44.

In some embodiments, the TNF inhibitor comprises or consists of:
(i) a heavy chain comprising or consisting of an amino acid sequence having at least 70%, 75%, 80%, 85%, 90%, 95%, 96%, 97%, 98%, 99%, or 100% identity to SEQ ID NO: 42, wherein the amino acid sequence comprises a HCDR1 having an amino acid sequence of SEQ ID NO: 34, a HCDR2 having an amino acid sequence of SEQ ID NO: 35, and a HCDR3 having an amino acid sequence of SEQ ID NO: 36; and/or
(ii) a light chain comprising or consisting of an amino acid sequence having at least 70%, 75%, 80%, 85%, 90%, 95%, 96%, 97%, 98%, 99%, or 100% identity to SEQ ID NO: 44, wherein the amino acid sequence comprises a LCDR1 having an amino acid sequence of SEQ ID NO: 34, a LCDR2 having an amino acid sequence of SEQ ID NO: 35, and a LCDR3 having an amino acid sequence of SEQ ID NO: 36.

| Name | Example sequence |
|---|---|
| Certolizumab HCDR1 | VFTDYG (SEQ ID NO: 34) |
| Certolizumab HCDR2 | NTYIGEPI (SEQ ID NO: 35) |
| Certolizumab HCDR3 | GYRSYAM (SEQ ID NO: 36) |
| Certolizumab LCDR1 | NVGTNVA (SEQ ID NO: 37) |
| Certolizumab LCDR2 | YSASFLY (SEQ ID NO: 38) |
| Certolizumab LCDR3 | QYNIY (SEQ ID NO: 39) |
| Certolizumab VH | EVQLVESGGGLVQPGGSLRLSCAASGYVFTDYGMNWVRQAPGKGLEWMGW |
| | INTYIGEPIYADSVKGRFTFSLDTSKSTAYLQMNSLRAEDTAVYYCARGY |
| | RSYAMDYWGQGTLVTVSS (SEQ ID NO: 40) |
| Certolizumab VL | DIQMTQSPSSLSASVGDRVTITCKASQNVGTNVAWYQQKPGKAPKALIYS |
| | ASFLYSGVPYRFSGSGSGTDFTLTISSLQPEDFATYYCQQYNIYPLTFGQ |
| | GTKVEIK (SEQ ID NO: 41) |
| Certolizumab VH + | EVQLVESGGGLVQPGGSLRLSCAASGYVFTDYGMNWVRQAPGKGLEWMGW |
| CH1 | INTYIGEPIYADSVKGRFTFSLDTSKSTAYLQMNSLRAEDTAVYYCARGY |
| | RSYAMDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKKV (SEQ ID NO: 42) |
| Certolizumab heavy | EVQLVESGGGLVQPGGSLRLSCAASGYVFTDYGMNWVRQAPGKGLEWMGW |
| chain | INTYIGEPIYADSVKGRFTFSLDTSKSTAYLQMNSLRAEDTAVYYCARGY |
| | RSYAMDYWGQGTLVTVSSASTKGPSVFPLAPSSKSTSGGTAALGCLVKDY |
| | FPEPVTVSWNSGALTSGVHTFPAVLQSSGLYSLSSVVTVPSSSLGTQTYI |
| | CNVNHKPSNTKVDKKVEPKSCDKTHTCAA (SEQ ID NO: 43) |
| Certolizumab light | DIQMTQSPSSLSASVGDRVTITCKASQNVGTNVAWYQQKPGKAPKALIYS |
| chain | ASFLYSGVPYRFSGSGSGTDFTLTISSLQPEDFATYYCQQYNIYPLTFGQ |
| | GTKVEIKRTVAAPSVFIFPPSDEQLKSGTASVVCLLNNFYPREAKVQWKV |
| | DNALQSGNSQESVTEQDSKDSTYSLSSTLTLSKADYEKHKVYACEVTHQG |
| | LSSPVTKSFNRGEC (SEQ ID NO: 44) |

### TNFR Domains

In some embodiments, the TNF inhibitor comprises the TNF-binding domain of a TNF receptor (TNFR).

TNF signals through two receptors (TNFR1 and TNFR2) that share a similar structural arrangement with an N-terminal extracellular domain (ECD) composed of four cysteine-rich domains (CRDs), an α-helical transmembrane domain and a cytoplasmic domain. The two receptors are most divergent in the cytoplasmic domain, where TNFR1 has a death domain that is absent from TNFR2 (see e.g. Bodmer, J. L., et al. Trends in biochemical sciences, 27 (1), pp. 19-26)

In some embodiments, the TNF inhibitor comprises a soluble form of a TNF receptor (TNFR). In some embodiments, the TNF inhibitor comprises a soluble form of TNFR1 or TNFR2. In some embodiments, the TNF inhibitor comprises or consists a soluble form of TNFR2.

The TNF-binding domain and/or soluble form of a TNF receptor may be fused to any suitable domain. Suitably, the TNF-binding domain and/or soluble form of a TNF receptor may be coupled to an Fc domain (e.g. the Fc portion of human IgG1).

### Etanercept

In some embodiments, the TNF inhibitor is etanercept or a derivative thereof.

Etanercept (Enbrel®) is a fusion protein, consisting of a TNFR2 domain coupled to the Fc portion of human IgG1. The skilled person would be able to generate etanercept derivatives using conservative mutations and/or knowledge of the mechanism of etanercept inhibition of TNF (see e.g. Lamanna, W. C., et al., 2017. Scientific reports, 7 (1), pp. 1-8).

In some embodiments, the TNF inhibitor comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 45. In some embodiments, the TNF inhibitor comprises or consists of the amino acid sequence of SEQ ID NO: 45.

Example etanercept sequence
(SEQ ID NO: 45)
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGDEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

### Other TNF Inhibitors

In some embodiments, the TNF inhibitor is a fusion protein, consisting of a TNFR1 extracellular domain coupled to the Fc portion of human IgG1.

In some embodiments, the TNF inhibitor comprises or consists of an amino acid sequence having at least 70%, at least 75%, at least 80%, at least 85%, at least 90%, at least 95%, at least 96%, at least 97%, at least 98%, at least 99% identity, or 100% identity to SEQ ID NO: 93. In some embodiments, the TNF inhibitor comprises or consists of the amino acid sequence of SEQ ID NO: 93.

Example fusion protein sequence
(SEQ ID NO: 93)
MGLSTVPDLLLPLVLLELLVGIYPSGVIGLVPHLGDREKRDSVCPQGKY
IHPQNNSICCTKCHKGTYLYNDCPGPGQDTDCRECESGSFTASENHLRH
CLSCSKCRKEMGQVEISSCTVDRDTVCGCRKNQYRHYWSENLFQCFNCS
LCLNGTVHLSCQEKQNTVCTCHAGFFLRENECVSCSNCKKSLECTKLCL
PQIENVKGTEDSGTTDKTHTCPPCPAPELLGGPSVFLFPPKPKDTLMIS
RTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSD
GSFFLYSKLTVDKSRWQQGNVFSCSVMHEALHNHYTQKSLSL

### Signal Peptides

The TNF inhibitor may be operably linked to one or more signal peptides.

A “signal peptide” may refer to a short peptide that directs the insertion of proteins into the membrane of the endoplasmic reticulum. Signal peptides are typically N-terminal extensions of newly synthesized secretory and membrane proteins that are 16 to 30 amino acid residues in length and comprised of a hydrophilic, usually positively charged N-terminal region, a central hydrophobic domain, and a C-terminal region with the cleavage site for signal peptidase. Besides these common characteristics, signal peptides do not share sequence similarity and some are more than 50 amino acid residues long (see e.g. Kapp, K., et al., 2009. Protein transport into the endoplasmic reticulum, pp. 1-16).

The TNF inhibitor may be operably linked to any suitable signal peptide(s). The SPdb signal peptide database is a repository of experimentally determined and computationally predicted signal peptides (see e.g. Choo, K. H., et al., 2005. BMC bioinformatics, 6 (1), pp. 1-8). Suitable signal peptides include, a Human Growth Hormone (HGH) signal peptide, an interleukin-2 (IL-2) signal peptide, a CD5 signal peptide, an immunoglobulin Kappa light chain signal peptide, a trypsinogen signal peptide, a serum albumin signal peptide, or a prolactin signal peptide.

In some embodiments, the TNF inhibitor is operably linked to one or more signal peptide selected from any of: a Human Growth Hormone (HGH) signal peptide, an interleukin-2 (IL-2) signal peptide, a CD5 signal peptide, an immunoglobulin Kappa light chain signal peptide, a trypsinogen signal peptide, a serum albumin signal peptide, and a prolactin signal peptide. In some embodiments, the TNF inhibitor is operably linked to one or more Human Growth Hormone (HGH) signal peptide.

In some embodiments, the TNF inhibitor is operably linked to one or more signal peptide comprising or consisting of an amino acid sequence having at least 70%, at least 75%, at least 80%, at least 85%, at least 90%, at least 95%, at least 96%, at least 97%, at least 98%, at least 99% identity, or 100% identity to SEQ ID NO: 46. In some embodiments the TNF inhibitor is operably linked to one or more signal peptide comprising or consisting of the amino acid sequence of SEQ ID NO: 46.

Example HGH signal peptide
(SEQ ID NO: 46)
MATGSRTSLLLAFGLLCLPWLQEGSA

### Example Nucleotide Sequences

The TNF inhibitor may be encoded by any suitable nucleotide sequence.

In some embodiments, the nucleotide sequence is codon-optimised, for example codon-optimised for expression in humans. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

In preferred embodiments, the TNF inhibitor is an anti-TNF antigen-binding fragment (Fab). Suitably, a nucleotide sequence encoding an anti-TNF Fab may comprise from 5′ to 3′: a first signal sequence; a nucleotide encoding a heavy chain; a linker sequence; a second signal sequence; and a nucleotide encoding a light chain. Suitably, a nucleotide sequence encoding an anti-TNF Fab may comprise from 5′ to 3′: a first signal sequence; a nucleotide encoding a light chain; a linker sequence; a second signal sequence; and a nucleotide encoding a heavy chain.

### Heavy Chains and Light Chains

The nucleotide encoding a heavy chain and the nucleotide encoding a light chain may encode any heavy chain and light chain combination described herein. For example, the nucleotide sequence encoding an anti-TNF Fab may comprise from 5′ to 3′:
(a) a first signal sequence; a nucleotide encoding an adalimumab heavy chain, or a derivative thereof; a linker sequence; a second signal sequence; and a nucleotide encoding an adalimumab light chain, or a derivative thereof;
(b) a first signal sequence; a nucleotide encoding an infliximab heavy chain, or a derivative thereof; a linker sequence; a second signal sequence; and a nucleotide encoding an infliximab light chain, or a derivative thereof;
(c) a first signal sequence; a nucleotide encoding a golimumab heavy chain, or a derivative thereof; a linker sequence; a second signal sequence; and a nucleotide encoding a golimumab light chain, or a derivative thereof;
(d) a first signal sequence; a nucleotide encoding a certolizumab heavy chain, or a derivative thereof; a linker sequence; a second signal sequence; and a nucleotide encoding a certolizumab light chain, or a derivative thereof;
(e) a first signal sequence; a nucleotide encoding an adalimumab light chain, or a derivative thereof; a linker sequence; a second signal sequence; and a nucleotide encoding an adalimumab heavy chain, or a derivative thereof;
(f) a first signal sequence; a nucleotide encoding an infliximab light chain, or a derivative thereof; a linker sequence; a second signal sequence; and a nucleotide encoding an infliximab heavy chain, or a derivative thereof;
(g) a first signal sequence; a nucleotide encoding a golimumab light chain, or a derivative thereof; a linker sequence; a second signal sequence; and a nucleotide encoding a golimumab heavy chain, or a derivative thereof; or
(h) a first signal sequence; a nucleotide encoding a certolizumab light chain, or a derivative thereof; a linker sequence; a second signal sequence; and a nucleotide encoding a certolizumab heavy chain, or a derivative thereof.

In some embodiments, the nucleotide sequence encoding an anti-TNF Fab may comprise from 5′ to 3′: a first signal sequence; a nucleotide encoding an adalimumab heavy chain, or a derivative thereof; a linker sequence; a second signal sequence; and a nucleotide encoding an adalimumab light chain, or a derivative thereof. In some embodiments, the nucleotide sequence encoding an anti-TNF Fab may comprise from 5′ to 3′: a first signal sequence; a nucleotide encoding an adalimumab light chain, or a derivative thereof; a linker sequence; a second signal sequence; and a nucleotide encoding an adalimumab heavy chain, or a derivative thereof.

In some embodiments, the nucleotide sequence encoding an adalimumab heavy chain comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 47. In some embodiments, the nucleotide sequence encoding an adalimumab heavy chain comprises or consists of SEQ ID NO: 47.

Example nucleotide sequence encoding an
adalimumab heavy chain
(SEQ ID NO: 47)
gaggtgcaactggtggaaagcggcggcggcctggtccagcctggaaggt
ccctgagactgagctgtgccgccagcggctttaccttcgacgactacgc
catgcactgggtgcgccaggcccctggcaagggcctggaatgggtctcc
gccatcacctggaatagcggccacatcgactacgccgatagcgtggaag
gcagattcaccatcagccgggacaacgccaagaactctctgtatctgca
aatgaacagcctgcgggctgaagatacagccgtgtactattgcgccaaa
gtgagctacctctccaccgccagcagcctggactattggggacagggca
ccctggtgaccgtgtctagcgcctccacaaagggcccttctgtgtttcc
actggctccaagctccaaaagcacatctggaggaaccgctgccctgggc
tgcctggttaaggactacttccccgagcctgtgaccgtgagctggaaca
gcggcgccctgacatctggtgttcataccttccctgccgttctgcaatc
ttctggactctacagcctgtcttctgtggtgaccgtgcccagcagcagc
cttggaacacagacctacatctgcaatgtgaaccacaagcctagcaaca
ccaaggtggacaagaaggtg

In some embodiments, the nucleotide sequence encoding an adalimumab light chain comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 48. In some embodiments, nucleotide sequence encoding an adalimumab light chain comprises or consists of SEQ ID NO: 48.

Example nucleotide sequence encoding an
adalimumab light chain
(SEQ ID NO: 48)
gacatccagatgacccagagcccttcctcactgagcgccagcgtgggc
gacagagtgactattacatgcagagccagccaaggcatccggaactac
ctggcctggtatcagcagaagcccggcaaagcccctaagctgctgatc
tacgccgccagcaccctgcaaagcggcgtgcctagcagattcagcggc
tcaggctctggcactgatttcaccctgaccatctcctctctgcaacct
gaggacgtggccacatactactgccagagatacaacagagccccatac
acctttggccagggcacaaaagtggaaatcaagagaaccgtggccgct
cccagtgtgttcatcttcccccccagtgatgagcagctgaagtccggc
acagcctctgtcgtgtgcctgctgaacaacttctaccccagagaggcc
aaggtgcagtggaaggtggataatgccctgcaaagcggcaacagccag
gagagcgtgacagagcaggacagcaaggacagcacctacagcctctct
agcacactgaccctgagcaaggccgactacgagaagcacaaggtgtac
gcatgcgaggtgacccaccagggcctgagcagtcctgtgaccaagagc
ttcaaccggggcgagtgt

In some embodiments, the nucleotide sequence encoding an anti-TNF Fab may comprise from 5′ to 3′: a first signal sequence; a nucleotide encoding an infliximab heavy chain, or a derivative thereof; a linker sequence; a second signal sequence; and a nucleotide encoding an infliximab light chain, or a derivative thereof. In some embodiments, the nucleotide sequence encoding an anti-TNF Fab may comprise from 5′ to 3′: a first signal sequence; a nucleotide encoding an infliximab light chain, or a derivative thereof; a linker sequence; a second signal sequence; and a nucleotide encoding an infliximab heavy chain, or a derivative thereof.

In some embodiments, the nucleotide sequence encoding an infliximab heavy chain comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 49. In some embodiments, nucleotide sequence encoding an infliximab light chain comprises or consists of SEQ ID NO: 49.

Example nucleotide sequence encoding an
infliximab heavy chain
(SEQ ID NO: 49)
gaggtgaaactggaggaaagtggaggaggcctcgttcaaccaggcggct
ccatgaagctgtcgtgtgtggcatctggcttcatcttcagcaatcactg
gatgaactgggtcaggcaatctcctgagaaggggctagagtgggtggcg
gagatccgctcaaaatcaatcaattccgccacacattatgcagagtcag
taaaagggcggttcaccatttctagagatgacagcaaaagcgccgtgta
cctccagatgaccgacctgcgaacagaggacactggggtctactactgc
tcccggaactactatggctccacctatgactactggggccaagggacca
cattgacagtatcctcagcctccactaaaggtccttcagtgtttccgct
ggctccctcctccaaaagtacgtcaggcggcaccgctgctctgggctgt
ctggtgaaggattacttccctgaacctgtgactgtttcctggaacagtg
gagccttgacttcaggagtccacacatttccggcagtgctccagagcag
tggtctctattccctaagcagtgtagtgaccgtgccctctagcagcctc
ggaacccagacatacatctgcaatgtcaatcacaagccaagcaatacaa
aagtggacaagaaggtt

In some embodiments, the nucleotide sequence encoding an infliximab heavy chain comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 50. In some embodiments, nucleotide sequence encoding an infliximab light chain comprises or consists of SEQ ID NO: 50.

Example nucleotide sequence encoding an
infliximab light chain
(SEQ ID NO: 50)
gacattctcctgacccagtcccctgctatcttgtctgtctcccccggag
agcgcgtctccttctcttgcagagcttcccagtttgtgggcagcagcat
tcactggtatcagcagagaacaaatggatcaccaaggcttttgatcaag
tatgcttcagaaagcatgagtgggataccatccaggtttagtggaagtg
gctctggtactgacttcactctctctataaacacggtggaaagcgaaga
tattgctgactattactgtcagcaaagccatagctggccatttactttt
ggatcagggaccaacctggaagtcaagagaactgtggccgcgccttcgg
tttttattttccccccatctgatgaacagctgaagagcggtacagccag
tgtagtgtgcctgctcaacaacttctaccctagagaagccaaggtgcag
tggaaggtcgacaatgcattacagagcgggaacagccaggaaagtgtta
ctgagcaggatagcaaggacagcacctactctctgtctagcacactcac
tttgtctaaagcagattatgagaaacataaagtttatgcctgtgaagtt
acccaccagggcctgagcagtcccgtcaccaagtctttcaaccgcgggg
agtgc

### Signal Sequences

The first signal sequence and the second signal sequence may encode the same signal peptide or different signal peptides. In some embodiments, the first signal sequence and the second signal sequence encode the same signal peptide. The first signal sequence and the second signal sequence may be any signal sequence described herein.

In some embodiments, the first signal sequence and/or the second signal sequence encode any of: a Human Growth Hormone (HGH) signal peptide, an interleukin-2 (IL-2) signal peptide, a CD5 signal peptide, an immunoglobulin Kappa light chain signal peptide, a trypsinogen signal peptide, a serum albumin signal peptide, and a prolactin signal peptide.

In some embodiments, the first signal sequence and/or the second signal sequence encode a Human Growth Hormone (HGH) signal peptide. In some embodiments, the first signal sequence and the second signal sequence encode a Human Growth Hormone (HGH) signal peptide.

In some embodiments, the first signal sequence and/or the second signal sequence comprises or consists of a nucleotide sequence having at least 70%, at least 75%, at least 80%, at least 85%, at least 90%, at least 95%, at least 96%, at least 97%, at least 98%, at least 99% identity, or 100% identity to any of SEQ ID NOs: 51-54. In some embodiments, the first signal sequence and/or the second signal sequence comprises or consists of the nucleotide sequence of any of SEQ ID NOs: 51-54.

Example HGH signal sequence
(SEQ ID NO: 51)
atggcaacaggtagtcgaaccagcctattactggccttcggtctcctgt
gtctgccctggcttcaagagggctctgc
Example HGH signal sequence
(SEQ ID NO: 52)
atggccacgggaagtcggacttccttactactcgcctttggtcttcttt
gcttgccatggctccaggagggtagtgca
Example HGH signal sequence
(SEQ ID NO: 53)
atggccacaggctctcggaccagcctgctgctggccttcggcctgctgt
gtctgccttggctgcaagagggcagcgcc
Example HGH signal sequence
(SEQ ID NO: 54)
atggctaccggcagcagaaccagcctgctgctggcattcggccttctgt
gcctgccttggctgcaagagggctctgcc

### Linker Sequences

The linker sequence may comprise one or more cleavage sites. As used herein, “cleavage sites” may include nucleotide sequences encoding specific peptide sequences at which site-specific proteases may cleave or cut the peptide (also known as enzymatically cleavable peptide motifs) and nucleotide sequences encoding self-cleaving peptides.

In preferred embodiments, the linker sequence encodes a cleavage site. In some embodiments, the linker sequence encodes a self-cleaving peptide and/or an enzymatically cleavable peptide motif.

In some embodiments, the linker sequence encodes a 2A self-cleaving peptide. 2A self-cleaving peptides are a class of 18-22 aa-long peptides, which can induce ribosomal skipping during translation of a protein in a cell. Suitable 2A self-cleaving peptides include T2A, P2A, E2A and F2A, or derivatives thereof.

In some embodiments, the linker sequence encodes a 2A self-cleaving peptide having at least 70%, at least 75%, at least 80%, at least 85%, at least 90%, at least 95%, at least 96%, at least 97%, at least 98%, at least 99% identity, or 100% identity to any of SEQ ID NOs: 55-58. In some embodiments, the linker sequence encodes a 2A self-cleaving peptide comprising or consisting of any of SEQ ID NOs: 55-58.

In some embodiments, the linker sequence encodes a 2A self-cleaving peptide having at least 70%, at least 75%, at least 80%, at least 85%, at least 90%, at least 95%, at least 96%, at least 97%, at least 98%, at least 99% identity, or 100% identity to SEQ ID NO: 55. In some embodiments, the linker sequence encodes a 2A self-cleaving peptide comprising or consisting of SEQ ID NO: 55.

Example P2A peptide sequence
(SEQ ID NO: 55)
ATNFSLLKQAGDVEENPGP
Example T2A peptide sequence
(SEQ ID NO: 56)
EGRGSLLTCGDVEENPGP
Example E2A peptide sequence
(SEQ ID NO: 57)
QCTNYALLKLAGDVESNPGP
Example F2A peptide sequence
(SEQ ID NO: 58)
VKQTLNFDLLKLAGDVESNPGP

In some embodiments, the linker sequence comprises a nucleotide sequence having at least 70%, at least 75%, at least 80%, at least 85%, at least 90%, at least 95%, at least 96%, at least 97%, at least 98%, at least 99% identity, or 100% identity to any of SEQ ID NOs: 59-60. In some embodiments, the linker sequence comprises any of SEQ ID NOs: 59-60.

Example P2A nucleotide sequence
(SEQ ID NO: 59)
gctacgaatttttcattgctcaagcaagcgggagatgtggaggagaac
cctggcccc
Example P2A nucleotide sequence
(SEQ ID NO: 60)
gctaccaacttcagcctcctgaaacaggccggcgatgtggaggaaaac
cctggacct

The linker sequence may comprise any other suitable nucleotide sequences, for example nucleotide sequences which aid expression of the anti-TNF Fab. Suitably, the linker sequence may comprise a furin site and/or a fusion protein linker sequence.

In some embodiments, the linker sequence comprises a furin site. Furin is a protease enzyme that may cleave at a conserved polybasic RNRR site. Suitably, the furin site encodes RKRR. In some embodiments, the linker sequence comprises fusion protein linker sequence. Fusion protein linker sequences may join two protein domains together. Suitably, the fusion protein linker sequence encodes SGSG.

In some embodiments, the linker sequence encodes from 5′ to 3′: a furin site, a fusion protein linker sequence, and a 2A self-cleaving peptide.

In some embodiments, the linker sequence encodes an amino acid having at least 70%, at least 75%, at least 80%, at least 85%, at least 90%, at least 95%, at least 96%, at least 97%, at least 98%, at least 99% identity, or 100% identity to SEQ ID NO: 61. In some embodiments, the linker sequence encodes an amino acid comprising or consisting of SEQ ID NO: 61.

Example linker peptide sequence
(SEQ ID NO: 61)
RKRRSGSGATNFSLLKQAGDVEENPGP

In some embodiments, the linker sequence comprises or consists of a nucleotide sequence having at least 70%, at least 75%, at least 80%, at least 85%, at least 90%, at least 95%, at least 96%, at least 97%, at least 98%, at least 99% identity, or 100% identity to any of SEQ ID NOs: 62-63. In some embodiments, the linker sequence comprises or consists of any of SEQ ID NOs: 62-63.

Example linker sequence
(SEQ ID NO: 62)
aggaagcggcgttccgggagcggggctacgaatttttcattgctcaag
caagcgggagatgtggaggagaaccctggcccc
Example linker sequence
(SEQ ID NO: 63)
agaaagcggagaagcggaagcggtgctaccaacttcagcctcctgaaa
caggccggcgatgtggaggaaaaccctggacct

### Other Sequences

The nucleotide sequence encoding an anti-TNF Fab may comprise any other suitable sequences. For example, the anti-TNF Fab may comprise a HA tag for detection. Suitably, an HA tag may comprise or consist of SEQ ID NO: 64.

Example HA tag
(SEQ ID NO: 64)
YPYDVPDYA

### Example Anti-TNF Fab Sequences

In some embodiments, the nucleotide sequence encoding an anti-TNF Fab encodes an amino acid sequence comprising or consisting of from 5′ to 3′: an amino acid sequence having at least 70% identity to SEQ ID NO: 46, an amino acid sequence having at least 70% identity to SEQ ID NO: 7, an amino acid sequence having at least 70% sequence identity to any of SEQ ID NOs: 55-58, an amino acid sequence having at least 70% identity to SEQ ID NO: 46, and an amino acid sequence having at least 70% identity to SEQ ID NO: 8.

In some embodiments, the nucleotide sequence encoding an anti-TNF Fab encodes an amino acid sequence having at least 70%, at least 75%, at least 80%, at least 85%, at least 90%, at least 95%, at least 96%, at least 97%, at least 98%, at least 99% identity, or 100% identity to SEQ ID NO: 65. In some embodiments, the nucleotide sequence encoding an anti-TNF Fab encodes the amino acid sequence of SEQ ID NO: 65.

Example adalimumab Fab amino acid sequence
(SEQ ID NO: 65)
MATGSRTSLLLAFGLLCLPWLQEGSAEVOLVESGGGLVQPGRSLRLSCAA
SGFTEDDYAMHWVRQAPGKGLEWVSAITWNSGHIDYADSVEGRFTISRDN
AKNSLYLQMNSLRAEDTAVYYCAKVSYLSTASSLDYWGQGTLVTVSSAST
KGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVRKRRS
GSGATNFSLLKQAGDVEENPGPMATGSRTSLLLAFGLLCLPWLQEGSADI
QMTQSPSSLSASVGDRVTITCRASQGIRNYLAWYQQKPGKAPKLLIYAAS
TLQSGVPSRESGSGSGTDETLTISSLQPEDVATYYCQRYNRAPYTFGQGT
KVEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSENRGEC

In some embodiments, the nucleotide sequence encoding an anti-TNF Fab comprises or consists of from 5′ to 3′: a nucleotide sequence having at least 70% identity to any of SEQ ID NOs: 51-54, a nucleotide sequence having at least 70% identity to SEQ ID NO: 47, a nucleotide sequence having at least 70% sequence identity to SEQ ID NO: 59 or 60, a nucleotide sequence having at least 70% identity to any of SEQ ID NOs: 51-54, and a nucleotide sequence having at least 70% identity to SEQ ID NO: 48.

In some embodiments, the nucleotide sequence encoding an anti-TNF Fab comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 66. In some embodiments, the nucleotide sequence encoding an anti-TNF Fab comprises or consists of SEQ ID NO: 66.

Example adalimumab Fab nucleotide sequence
(SEQ ID NO: 66)
atggccacaggctctcggaccagcctgctgctggccttcggcctgctgtg
tctgccttggctgcaagagggcagcgccgaggtgcaactggtggaaagcg
gcggcggcctggtccagcctggaaggtccctgagactgagctgtgccgcc
agcggctttaccttcgacgactacgccatgcactgggtgcgccaggcccc
tggcaagggcctggaatgggtctccgccatcacctggaatagcggccaca
tcgactacgccgatagcgtggaaggcagattcaccatcagccgggacaac
gccaagaactctctgtatctgcaaatgaacagcctgcgggctgaagatac
agccgtgtactattgcgccaaagtgagctacctctccaccgccagcagcc
tggactattggggacagggcaccctggtgaccgtgtctagcgcctccaca
aagggcccttctgtgtttccactggctccaagctccaaaagcacatctgg
aggaaccgctgccctgggctgcctggttaaggactacttccccgagcctg
tgaccgtgagctggaacagcggcgccctgacatctggtgttcataccttc
cctgccgttctgcaatcttctggactctacagcctgtcttctgtggtgac
cgtgcccagcagcagccttggaacacagacctacatctgcaatgtgaacc
acaagcctagcaacaccaaggtggacaagaaggtgagaaagcggagaagc
ggaagcggtgctaccaacttcagcctcctgaaacaggccggcgatgtgga
ggaaaaccctggacctatggctaccggcagcagaaccagcctgctgctgg
cattcggccttctgtgcctgccttggctgcaagagggctctgccgacatc
cagatgacccagagcccttcctcactgagcgccagcgtgggcgacagagt
gactattacatgcagagccagccaaggcatccggaactacctggcctggt
atcagcagaagcccggcaaagcccctaagctgctgatctacgccgccagc
accctgcaaagcggcgtgcctagcagattcagcggctcaggctctggcac
tgatttcaccctgaccatctcctctctgcaacctgaggacgtggccacat
actactgccagagatacaacagagccccatacacctttggccagggcaca
aaagtggaaatcaagagaaccgtggccgctcccagtgtgttcatcttccc
ccccagtgatgagcagctgaagtccggcacagcctctgtcgtgtgcctgc
tgaacaacttctaccccagagaggccaaggtgcagtggaaggtggataat
gccctgcaaagcggcaacagccaggagagcgtgacagagcaggacagcaa
ggacagcacctacagcctctctagcacactgaccctgagcaaggccgact
acgagaagcacaaggtgtacgcatgcgaggtgacccaccagggcctgagc
agtcctgtgaccaagagcttcaaccggggcgagtgt

In some embodiments, the nucleotide sequence encoding an anti-TNF Fab encodes an amino acid sequence comprising or consisting of from 5′ to 3′: an amino acid sequence having at least 70% identity to SEQ ID NO: 46, an amino acid sequence having at least 70% identity to SEQ ID NO: 18, an amino acid sequence having at least 70% sequence identity to any of SEQ ID NOs: 55-58, an amino acid sequence having at least 70% identity to SEQ ID NO: 46, and an amino acid sequence having at least 70% identity to SEQ ID NO: 19.

In some embodiments, the nucleotide sequence encoding an anti-TNF Fab encodes an amino acid sequence having at least 70%, at least 75%, at least 80%, at least 85%, at least 90%, at least 95%, at least 96%, at least 97%, at least 98%, at least 99% identity, or 100% identity to SEQ ID NO: 67. In some embodiments, the nucleotide sequence encoding an anti-TNF Fab encodes the amino acid sequence of SEQ ID NO: 67.

Example infliximab Fab amino acid sequence
(SEQ ID NO: 67)
MATGSRTSLLLAFGLLCLPWLQEGSAEVKLEESGGGLVQPGGSMKLSCVA
SGFIFSNHWMNWVRQSPEKGLEWVAEIRSKSINSATHYAESVKGRFTISR
DDSKSAVYLQMTDLRTEDTGVYYCSRNYYGSTYDYWGQGTTLTVSSASTK
GPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVRKRRSG
SGATNFSLLKQAGDVEENPGPMATGSRTSLLLAFGLLCLPWLQEGSADIL
LTQSPAILSVSPGERVSFSCRASQFVGSSIHWYQQRTNGSPRLLIKYASE
SMSGIPSRESGSGSGTDETLSINTVESEDIADYYCQQSHSWPFTFGSGTN
LEVKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSS
PVTKSENRGEC

In some embodiments, the nucleotide sequence encoding an anti-TNF Fab comprises or consists of from 5′ to 3′: a nucleotide sequence having at least 70% identity to any of SEQ ID NOs: 51-54, a nucleotide sequence having at least 70% identity to SEQ ID NO: 49, a nucleotide sequence having at least 70% sequence identity to SEQ ID NO: 59 or 60, a nucleotide sequence having at least 70% identity to any of SEQ ID NOs: 51-54, and a nucleotide sequence having at least 70% identity to SEQ ID NO: 50.

In some embodiments, the nucleotide sequence encoding an anti-TNF Fab comprises or consists of a nucleotide sequence having at least 70%, at least 75%, at least 80%, at least 85%, at least 90%, at least 95%, at least 96%, at least 97%, at least 98%, at least 99% identity, or 100% identity to SEQ ID NO: 68. In some embodiments, the nucleotide sequence encoding an anti-TNF Fab comprises or consists of SEQ ID NO: 68.

Example infliximab Fab nucleotide sequence
(SEQ ID NO: 68)
atggcaacaggtagtcgaaccagcctattactggccttcggtctcctgtg
tctgccctggcttcaagagggctctgctgaggtgaaactggaggaaagtg
gaggaggcctcgttcaaccaggcggctccatgaagctgtcgtgtgtggca
tctggcttcatcttcagcaatcactggatgaactgggtcaggcaatctcc
tgagaaggggctagagtgggtggcggagatccgctcaaaatcaatcaatt
ccgccacacattatgcagagtcagtaaaagggcggttcaccatttctaga
gatgacagcaaaagcgccgtgtacctccagatgaccgacctgcgaacaga
ggacactggggtctactactgctcccggaactactatggctccacctatg
actactggggccaagggaccacattgacagtatcctcagcctccactaaa
ggtccttcagtgtttccgctggctccctcctccaaaagtacgtcaggcgg
caccgctgctctgggctgtctggtgaaggattacttccctgaacctgtga
ctgtttcctggaacagtggagccttgacttcaggagtccacacatttccg
gcagtgctccagagcagtggtctctattccctaagcagtgtagtgaccgt
gccctctagcagcctcggaacccagacatacatctgcaatgtcaatcaca
agccaagcaatacaaaagtggacaagaaggttaggaagcggcgttccggg
agcggggctacgaatttttcattgctcaagcaagcgggagatgtggagga
gaaccctggccccatggccacgggaagtcggacttccttactactcgcct
ttggtcttctttgcttgccatggctccaggagggtagtgcagacattctc
ctgacccagtcccctgctatcttgtctgtctcccccggagagcgcgtctc
cttctcttgcagagcttcccagtttgtgggcagcagcattcactggtatc
agcagagaacaaatggatcaccaaggcttttgatcaagtatgcttcagaa
agcatgagtgggataccatccaggtttagtggaagtggctctggtactga
cttcactctctctataaacacggtggaaagcgaagatattgctgactatt
actgtcagcaaagccatagctggccatttacttttggatcagggaccaac
ctggaagtcaagagaactgtggccgcgccttcggtttttattttcccccc
atctgatgaacagctgaagagcggtacagccagtgtagtgtgcctgctca
acaacttctaccctagagaagccaaggtgcagtggaaggtcgacaatgca
ttacagagcgggaacagccaggaaagtgttactgagcaggatagcaagga
cagcacctactctctgtctagcacactcactttgtctaaagcagattatg
agaaacataaagtttatgcctgtgaagttacccaccagggcctgagcagt
cccgtcaccaagtctttcaaccgcggggagtgc

### Promoters

The vector of the present invention may comprise a promoter, preferably an inflammation-inducible promoter. Suitably, the promoter may be operably linked to the nucleotide sequence encoding a TNF inhibitor (e.g. anti-TNF antibody fragment). The term “operably linked” may mean that the components described are in a relationship permitting them to function in their intended manner.

A “promoter” may refer to a region of DNA that leads to initiation of transcription of a gene. Promoters are typically located near the transcription start sites of genes, upstream on the DNA (towards the 5′ region of the sense strand). Any suitable promoter may be used, the selection of which may be readily made by the skilled person.

A promoter typically comprises a “core” and a “proximal” region. The “core promoter region” may comprise promoter elements such as a transcription start site, RNA polymerase binding sites and general transcription factor binding sites (e.g. TATA box, B recognition element). The “proximal promoter region” may comprise primary regulatory elements and specific transcription factor binding sites which are required, for example, to facilitate effective and controllable transcription. The size and components of both the core and proximal promoter regions typically vary in a gene specific manner.

In some embodiments, the promoter is an ocular tissue-specific promoter. As used herein, an “ocular tissue-specific promoter” is a promoter which preferentially facilitates expression of a gene in ocular cells (e.g. photoreceptors, RPE cells, retinal ganglion cells etc.).

In other embodiments, the promoter is a constitutive promoter. As used herein, a “constitutive promoter” is a promoter which is always active. Exemplary constitutive promoters include a chicken beta-actin (CBA) promoter, or a variant or fragment thereof. The promoter may comprise or consist of a CAG promoter, or a variant or fragment thereof.

### Inflammation-Inducible Promoters

The vector of the present invention preferably comprises an inflammation-inducible promoter. Suitably, the nucleotide sequence encoding a TNF inhibitor (e.g. anti-TNF antibody fragment) is operably linked to the inflammation-inducible promoter. The promoter may facilitate expression of the TNF inhibitor in response to inflammation.

The use of an inflammation-inducible promoter is advantageous because the TNF inhibitor may be expressed when inflammation occurs at a sub-clinical level and thereby prevent damage caused by flare-ups, before symptoms occur.

As used herein, an “inflammation-inducible promoter” may refer to a promoter which preferentially facilitates expression of an operably-linked transgene in response to inflammation. Inflammation may be characterised by, at a tissue level, redness, swelling, heat, pain and/or loss of tissue function (see e.g. Chen, L., et al., 2018. Oncotarget, 9 (6), p. 7204). Inflammation may be characterised by, at a cellular level, elevated levels of pro-inflammatory cytokines, activated immune cells, or acute phase proteins. Inflammation may be considered acute, i.e. an immediate response to harmful stimuli, or chronic, i.e. prolonged inflammation.

Suitably, an inflammation-inducible promoter may facilitate higher expression of an operably-linked transgene in response to higher levels of inflammation. Higher expression may be measured for example by measuring the expression of a transgene, e.g. green fluorescence protein (GFP), operably linked to the promoter, wherein expression of the transgene correlates with the ability of the promoter to facilitate expression of a gene. Levels of inflammation can be determined by suitable methods known in the art (see e.g. Menzel et al., 2021, Antioxidants, 10 (3), p. 414). For example, measurements of levels of markers of inflammation can be taken from appropriate media, such as body fluids. Appropriate body fluids may include blood, urine or vitreus humor.

Suitably, an inflammation-inducible promoter may facilitate higher expression of an operably-linked transgene in an ocular cell (e.g. photoreceptors, RPE cells, retinal ganglion cells etc.) in response to higher levels of inflammation in the ocular cell. The levels of inflammation in the eye may be determined by suitable non-invasive methods known in the art. For example, by grading ocular inflammation (see e.g. McNeil, R., 2016. Eye news, 22 (5), pp. Jan. 4, 2019) or by laser flare photometry (see e.g. Tugal-Tutkun, I. and Herbort, C. P., 2010. International ophthalmology, 30 (5), pp. 453-464).

The inflammation-inducible promoter may be (or may be derived from) a promoter associated with a gene with increased expression in response to inflammation. Suitably, the inflammation-inducible promoter may be (or may be derived from) a promoter associated with a gene with increased expression in ocular cells in response to inflammation (e.g. uveitis). For example, genes with increase expression in uveitis may include CCL16, IL15, CCL7, CYSLTR1, IL17, IL4, CCL8, IL15RA, CCR1, CDC25A, LAT, MEF2B, CSF3, MAP2K7, IL20, TYK2, MAF, COL1A1, IKBKG, MGC27165, IL10, CIITA, NFATC2IP, MEF2D, GSK3A, TH1L, IL3, IL10RB, SMAD9, C19orf10, IL13RA2, TGFB2, LEP, HAVCR2, SOCS5, IRF1, TXLNA, RFX2, FADD, TGIF, CCR4, RFX1, MIF, IL10RA, CXCL5, LTA, IGFBP3, NFkBIB, CXCL13, IKBKE, MEF2A, LAG3, ICAM1, AMHR2, CCL15, and PDGFB (see e.g. Li, Z., et al., 2008. The Journal of Immunology, 181 (7), pp. 5147-5157). Methods to identify promoters associated with genes will be known to those of skill in the art.

In some embodiments, the inflammation inducible promoter comprises an IFN-beta minimal promoter, or a fragment or derivative thereof. In some embodiments, the inflammation-inducible promoter comprises a nucleotide sequence which is at least 70% identical to SEQ ID NO: 69 or a fragment thereof. In some embodiments, the inflammation-inducible promoter comprises a nucleotide sequence which is at least 75%, at least 80%, at least 85%, at least 90%, at least 95%, at least 96%, at least 97%, at least 98%, or at least 99% identical to SEQ ID NO: 69 or a fragment thereof. In some embodiments, the inflammation-inducible promoter comprises SEQ ID NO: 69.

Example IFN-beta minimal promoter
(SEQ ID NO: 69)
agcttgaataaaatgaatattagaagctgttagaataagagaaaatgaca
gaggaaaactgaaagggagaactgaaagtgggaaattcctctgaggcaga
aaggaccatcccttataaatagcacaggccatgaaggaagatcattctca
ctgcagcctttgacagcctttgcctcatcttg

The inflammation-inducible promoter may comprise one or more inflammation-associated transcription factor binding motif. An “inflammation-associated transcription factor binding motif” or “inflammation-associated transcription factor binding site” may refer to a nucleotide sequence that an inflammation-associated transcription factor binds. Exemplary inflammation-associated transcription factors include but are not limited to AP-1, NF-κB, IRFs, STATs and NFAT (see e.g. Smale ST. Cell. 2010 Mar. 19; 140 (6): 833-44., Platanitis E, Decker T. Front Immunol. 2018 Nov. 13; 9:2542, Pessler F, Dai L, Cron RQ, Schumacher HR. Autoimmun Rev. 2006 February; 5 (2): 106-10). Exemplary inflammation-associated transcription factor binding motifs may include, but are not limited to: AP-1 binding motifs, NF-κB binding motifs (KB sites), the interferon-stimulated response element (ISRE), the gamma-interferon-activated sequence (GAS), and NFAT binding motifs.

In some embodiments, the inflammation-inducible promoter comprises at least one inflammation-associated transcription factor binding motif, which may be selected from an AP-1 transcription factor binding motif; a NF-κB transcription factor binding motif; an IRF transcription factor binding motif; a STAT transcription factor binding motif; and a NFAT transcription factor binding motif; or any combination thereof.

In some embodiments, the inflammation-inducible promoter comprises two or more inflammation-associated transcription factor binding motifs. In some embodiments, the inflammation-inducible promoter comprises three or more, four or more, five or more, six or more, seven or more, eight or more, nine or more, or ten or more inflammation-associated transcription factor binding motifs.

### AP-1 Binding Motifs

In some embodiments, the inflammation-inducible promoter comprises one or more AP-1 binding motif.

Activator protein 1 (AP-1) is a transcription factor that regulates gene expression in response to a variety of stimuli, including cytokines, growth factors, stress, and bacterial and viral infections. An “AP-1 binding motif”, also known as an “AP-1 transcription factor binding motif” or an “AP-1 promoter site”, is a DNA sequence to which AP-1 transcription factors are able to bind (see e.g. Kim, H., et al., 1997. Biochemical Journal, 324 (2), pp. 547-553).

In some embodiments, the inflammation-inducible promoter comprises two or more AP-1 binding motifs. In some embodiments, the inflammation-inducible promoter comprises three or more AP-1 binding motifs. In some embodiments, the inflammation-inducible promoter comprises four or more AP-1 binding motifs. In some embodiments, the inflammation-inducible promoter comprises five or more AP-1 binding motifs. In some embodiments, the inflammation-inducible promoter comprises five AP-1 binding motifs.

Exemplary AP-1 binding motifs are shown as SEQ ID NO: 70 (where “s” is g or c and “m” is a or c), SEQ ID NO: 71, SEQ ID NO: 72, and SEQ ID NO: 73. Any other variant or derivative to which AP-1 binds may be used in the present invention.

In some embodiments, the inflammation-inducible promoter comprises the nucleotide sequence SEQ ID NO: 70.

| | Example AP-1 binding consensus motif |
|---|---|
| (SEQ ID NO: 70) | |
| | tgastma |

In some embodiments, the inflammation-inducible promoter comprises the nucleotide sequence SEQ ID NO: 71, or a derivative thereof having one or two nucleotide substitutions.

| | Example AP-1 binding motif 1 |
|---|---|
| (SEQ ID NO: 71) | |
| | tgagtca |

In some embodiments, the inflammation-inducible promoter comprises the nucleotide sequence SEQ ID NO: 72, or a derivative thereof having one or two nucleotide substitutions.

| | Example AP-1 binding motif 2 |
|---|---|
| (SEQ ID NO: 72) | |
| | tgactca |

In some embodiments, the inflammation-inducible promoter comprises the nucleotide sequence SEQ ID NO: 73, or a derivative thereof having one or two nucleotide substitutions.

| | Example AP-1 binding motif 3 |
|---|---|
| (SEQ ID NO: 73) | |
| | tgactaa |

### NF-kB Binding Motifs

In some embodiments, the inflammation-inducible promoter comprises one or more NF-kB binding motif.

Nuclear factor kappa-light-chain-enhancer of activated B cells (NF-kB) is a protein complex that controls transcription of DNA in response to stimuli such as stress, cytokines, free radicals, heavy metals, ultraviolet irradiation, oxidized LDL, and bacterial or viral antigens. An “NF-kB binding motif”, also known as an “NF-kB transcription factor binding motif” or an “NF-kB promoter site”, is a DNA sequence to which NF-kB transcription factors are able to bind (see e.g. Natoli, G., 2006. FEBS letters, 580 (12), pp. 2843-2849).

In some embodiments, the inflammation-inducible promoter comprises two or more NF-kB binding motifs. In some embodiments, the inflammation-inducible promoter comprises three or more NF-kB binding motifs. In some embodiments, the inflammation-inducible promoter comprises four or more NF-kB binding motifs. In some embodiments, the inflammation-inducible promoter comprises five or more NF-kB binding motifs. In some embodiments, the inflammation-inducible promoter comprises five NF-kB binding motifs.

Exemplary NF-kB binding motifs are shown as SEQ ID NO: 74 (where “r” is a purine, “y” is a pyrimidine and “n” is any nucleotide) and SEQ ID NO: 75. Any other variant or derivative to which NF-kB binds may be used in the present invention.

In some embodiments, the inflammation-inducible promoter comprises the nucleotide sequence SEQ ID NO: 74.

| | Example NF-KB binding consensus motif |
|---|---|
| (SEQ ID NO: 74) | |
| | gggrnyyycc |

In some embodiments, the inflammation-inducible promoter comprises the nucleotide sequence SEQ ID NO: 75, or a derivative thereof having one or two nucleotide substitutions.

| | Example NF-KB binding motif |
|---|---|
| (SEQ ID NO: 75) | |
| | ggggactttccact |

### Other Inflammation-Associated Transcription Factor Binding Motifs

In some embodiments, the inflammation-inducible promoter comprises one or more, two or more, three or more, four or more, or five or more interferon-stimulated response elements (ISREs). An ISRE (also known as an IRF transcription factor binding motif) is a DNA sequence to which IRF transcription factors are able to bind. All IRF family members possess an N-terminal DNA-binding domain that recognises the ISRE, which may be characterized by the consensus sequence AANNGAAA (see e.g. Yanai, H., et al., 2012. Oncoimmunology, 1 (8), pp. 1376-1386).

In some embodiments, the inflammation-inducible promoter comprises one or more, two or more, three or more, four or more, or five or more gamma-interferon-activated sequences (GASs). A GAS (also known as a “STAT transcription factor binding motif”) is a DNA sequence to which STAT transcription factors are able to bind. Several members of the STAT protein family in particular STAT1, STAT2, STAT3, STAT4 and STAT6 act as transcription factors in modulating inflammatory responses. STAT transcription factors bind to similar sequences and may have the palindromic core motif TTCN₂₋₄GAA (see e.g. Ehret, G. B., et al., 2001. Journal of Biological Chemistry, 276 (9), pp. 6675-6688).

In some embodiments, the inflammation-inducible promoter comprises one or more, two or more, three or more, four or more, or five or more NFAT binding motifs. A NFAT transcription factor binding motif is a DNA sequence to which NFAT transcription factors are able to bind. The NFAT family acts synergistically with AP-1 proteins on DNA elements which contain adjacent NFAT and AP-1 binding sites, to regulate the expression of inducible genes. An NFAT binding motif may be characterized by the consensus sequence TGGAAA (see e.g. Macian, F., et al., 2001. Oncogene, 20 (19), pp. 2476-2489).

### Example Inflammation-Inducible Promoters

In some embodiments, the inflammation-inducible promoter comprises a combination of two or more different inflammation-associated transcription factor binding motifs. For example, the inflammation-inducible promoter may comprise any combination of: one or more AP-1 binding motif, one or more NF-κB binding motifs, one or more gamma-interferon-activated sequence (GAS), one or more interferon-stimulated response element (ISRE), and one or more NFAT binding motif.

In some embodiments, the inflammation-inducible promoter comprises one or more AP-1 binding motifs and/or one or more NF-κB binding motifs. In some embodiments, the inflammation-inducible promoter comprises two or more AP-1 binding motifs and/or two or more NF-κB binding motifs. In some embodiments, the inflammation-inducible promoter comprises three or more AP-1 binding motifs and/or three or more NF-κB binding motifs. In some embodiments, the inflammation-inducible promoter comprises four or more AP-1 binding motifs and/or four or more NF-κB binding motifs. In some embodiments, the inflammation-inducible promoter comprises five or more AP-1 binding motifs and/or five or more NF-κB binding motifs.

The inflammation-associated transcription factor binding motifs may be coupled. The term “coupled” may mean that the inflammation-associated transcription factor binding motifs are in a relationship permitting them to function in their intended manner (e.g. to bind inflammation-associated transcription factor). Suitably, the inflammation-associated transcription factor binding motifs may be linked by short nucleotide sequences or may be directly linked, or any combination thereof. Suitably, the inflammation-associated transcription factor binding motifs are linked by linker sequences of from 1 to about 20 nucleotides, or from 1 to about 10 nucleotides. Suitably, the inflammation-associated transcription factor binding motifs are directly linked (i.e. with no linker sequence in between).

Suitably, the inflammation-inducible promoter may comprise one or more inflammation-associated transcription factor binding sites and a minimal promoter (e.g. an IFN-beta minimal promoter). Suitably, the inflammation-inducible promoter may comprise a proximal promoter region comprising one or more inflammation-associated transcription factor binding sites. Suitably, the inflammation-inducible promoter may comprise a proximal promoter region comprising one or more inflammation-associated transcription factor binding sites and a core promoter region derived from a gene with selective expression in response to inflammation.

In some embodiments, the inflammation-inducible promoter comprises or consists of a nucleotide sequence which is at least 70% identical to SEQ ID NO: 76, or a fragment thereof. Suitably the inflammation-inducible promoter comprises or consists of a nucleotide sequence which is at least 75%, at least 80%, at least 85%, at least 90%, at least 95%, at least 96%, at least 97%, at least 98%, or at least 99% identical to SEQ ID NO: 76 or a fragment thereof. In some embodiments, the inflammation-inducible promoter comprises or consists of the sequence SEQ ID NO: 76 or a fragment thereof.

Example inflammation-inducible promoter
(SEQ ID NO: 76)
tgagtcactgactcagtgagtcactgactcagtgagtaaggaattctggg
gactttccactggggactttccactggggactttccactggggactttcc
actggggactttccactcctgcaggagcttgaataaaatgaatattagaa
gctgttagaataagagaaaatgacagaggaaaactgaaagggagaactga
aagtgggaaattcctctgaggcagaaaggaccatcccttataaatagcac
aggccatgaaggaagatcattctcactgcagcctttgacagcctttgcct
catcttg

In some embodiments, a nucleotide sequence encoding a TNF inhibitor operably linked to an inflammation-inducible promoter comprises or consists of a nucleotide sequence which is at least 70% identical to SEQ ID NO: 77 or a fragment thereof. Suitably, the nucleotide sequence encoding a TNF inhibitor operably linked to an inflammation-inducible promoter comprises or consists of a nucleotide sequence which is at least 75%, at least 80%, at least 85%, at least 90%, at least 95%, at least 96%, at least 97%, at least 98%, or at least 99% identical to SEQ ID NO: 77 or a fragment thereof. In some embodiments, the nucleotide sequence encoding a TNF inhibitor operably linked to an inflammation-inducible promoter has a nucleotide sequence comprising or consisting of the sequence SEQ ID NO: 77 or a fragment thereof.

Example inflammation-inducible promoter and
adalimumab Fab nucleotide sequence
(SEQ ID NO: 77)
Tgagtcactgactcagtgagtcactgactcagtgagtaaggaattctggg
gactttccactggggactttccactggggactttccactggggactttcc
actggggactttccactcctgcaggagcttgaataaaatgaatattagaa
gctgttagaataagagaaaatgacagaggaaaactgaaagggagaactga
aagtgggaaattcctctgaggcagaaaggaccatcccttataaatagcac
aggccatgaaggaagatcattctcactgcagcctttgacagcctttgcct
catcttgcagatcctgcagaagttggtcgtgaggcactgggcaggtaagt
atcaaggttacaagacaggtttaaggagaccaatagaaactgggcttgtc
gagacagagaagactcttgcgtttctgataggcacctattggtcttactg
acatccactttgcctttctctccacaggtgtccaggcggccgccatggcc
acaggctctcggaccagcctgctgctggccttcggcctgctgtgtctgcc
ttggctgcaagagggcagcgccgaggtgcaactggtggaaagcggcggcg
gcctggtccagcctggaaggtccctgagactgagctgtgccgccagcggc
tttaccttcgacgactacgccatgcactgggtgcgccaggcccctggcaa
gggcctggaatgggtctccgccatcacctggaatagcggccacatcgact
acgccgatagcgtggaaggcagattcaccatcagccgggacaacgccaag
aactctctgtatctgcaaatgaacagcctgcgggctgaagatacagccgt
gtactattgcgccaaagtgagctacctctccaccgccagcagcctggact
attggggacagggcaccctggtgaccgtgtctagcgcctccacaaagggc
ccttctgtgtttccactggctccaagctccaaaagcacatctggaggaac
cgctgccctgggctgcctggttaaggactacttccccgagcctgtgaccg
tgagctggaacagcggcgccctgacatctggtgttcataccttccctgcc
gttctgcaatcttctggactctacagcctgtcttctgtggtgaccgtgcc
cagcagcagccttggaacacagacctacatctgcaatgtgaaccacaagc
ctagcaacaccaaggtggacaagaaggtgagaaagcggagaagcggaagc
ggtgctaccaacttcagcctcctgaaacaggccggcgatgtggaggaaaa
ccctggacctatggctaccggcagcagaaccagcctgctgctggcattcg
gccttctgtgcctgccttggctgcaagagggctctgccgacatccagatg
acccagagcccttcctcactgagcgccagcgtgggcgacagagtgactat
tacatgcagagccagccaaggcatccggaactacctggcctggtatcagc
agaagcccggcaaagcccctaagctgctgatctacgccgccagcaccctg
caaagcggcgtgcctagcagattcagcggctcaggctctggcactgattt
caccctgaccatctcctctctgcaacctgaggacgtggccacatactact
gccagagatacaacagagccccatacacctttggccagggcacaaaagtg
gaaatcaagagaaccgtggccgctcccagtgtgttcatcttcccccccag
tgatgagcagctgaagtccggcacagcctctgtcgtgtgcctgctgaaca
acttctaccccagagaggccaaggtgcagtggaaggtggataatgccctg
caaagcggcaacagccaggagagcgtgacagagcaggacagcaaggacag
cacctacagcctctctagcacactgaccctgagcaaggccgactacgaga
agcacaaggtgtacgcatgcgaggtgacccaccagggcctgagcagtcct
gtgaccaagagcttcaaccggggcgagtgt

### Other Regulatory Elements

The vector of the present invention may comprise one or more further regulatory elements which may act pre- or post-transcriptionally. Suitably, the nucleotide sequence encoding a TNF inhibitor is operably linked to one or more further regulatory elements which may act pre- or post-transcriptionally.

As used herein, a “regulatory element” may refer to any nucleotide sequence that facilitates expression of a polypeptide, e.g. acts to increase expression of a transcript or to enhance mRNA stability. Suitable regulatory elements include for example enhancer elements, post-transcriptional regulatory elements, introns, polyadenylation sites, and Kozak sequences.

### Enhancers

The vector of the present invention may comprise an enhancer. Suitably, the nucleotide sequence encoding a TNF inhibitor is operably linked to an enhancer. The enhancer may facilitate expression of the TNF inhibitor in ocular cells (e.g. retinal ganglion cells, RPE cells, photoreceptors, glial cells).

An “enhancer” or “enhancer element” may refer to a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

The vector of the present invention may comprise an ocular tissue-specific enhancer. Suitably, the enhancer may be operably linked to the nucleotide sequence encoding a TNF inhibitor.

As used herein, a “tissue-specific enhancer” is an enhancer which preferentially facilitates expression of a gene in specific cells or tissues. Suitably, a tissue-specific enhancer may facilitate higher expression of a gene in specific cells-types as compared to other cell-types. Higher expression may be measured for example by measuring the expression of a transgene, e.g. green fluorescence protein (GFP), operably linked to the enhancer, wherein expression of the transgene correlates with the ability of the enhancer to facilitate expression of a gene. Suitable tissue-specific enhancers will be known to those of skill in the art. The enhancer may be a retinal-specific enhancer, preferably a retinal ganglion-specific enhancer. Suitably, the enhancer may be (or may be derived from) an enhancer associated with a gene with selective expression in human retinal cells. Methods to identify the enhancer regions associated with genes will be known to those of skill in the art.

### Polyadenylation Sequences

The vector of the present invention may comprise a polyadenylation sequence. Suitably, the nucleotide sequence encoding a TNF inhibitor is operably linked to a polyadenylation sequence. A polyadenylation sequence may be inserted after the nucleotide sequence to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

Suitable polyadenylation sequences will be known to those of skill in the art (see e.g. Schambach, A., et al., 2007. Molecular Therapy, 15 (6), pp. 1167-1173; and Choi, J. H. et al., 2014. Molecular brain, 7 (1), pp. 1-10). Example polyadenylation sequences include the bovine growth hormone (bGH) polyadenylation sequence, the SV40 polyadenylation sequence, and the rabbit beta-globin polyadenylation sequence.

In some embodiments, the polyadenylation sequence comprises or consists of a nucleotide sequence which is at least 70% identical to SEQ ID NO: 78 or a fragment thereof. Suitably, the polyadenylation sequence comprises or consists of a nucleotide sequence which is at least 75%, at least 80%, at least 85%, at least 90%, at least 95%, at least 96%, at least 97%, at least 98%, or at least 99% identical to SEQ ID NO: 78 or a fragment thereof.

In some embodiments, the polyadenylation sequence comprises or consists of the nucleotide sequence SEQ ID NO: 78 or a fragment thereof.

Example bGH polyadenylation sequence
(SEQ ID NO: 78)
gcctcgactgtgccttctagttgccagccatctgttgtttgcccctcccc
cgtgccttccttgaccctggaaggtgccactcccactgtcctttcctaat
aaaatgaggaaattgcatcgcattgtctgagtaggtgtcattctattctg
gggggtggggtggggcaggacagcaagggggaggattgggaagacaatag
caggcatgctgggga

### Post-Transcriptional Regulatory Elements

The vector of the present invention may comprise a post-transcriptional regulatory element. Suitably, the nucleotide sequence encoding a TNF inhibitor is operably linked to a post-transcriptional regulatory elements.

The vector of the present invention may comprise a woodchuck hepatitis post-transcriptional regulatory element (WPRE). Suitably, the nucleotide sequence encoding a TNF inhibitor is operably linked to the WPRE.

Suitable WPRE sequences will be known to those of skill in the art (see e.g. Zufferey, R., et al., 1999. Journal of virology, 73 (4), pp. 2886-2892; and Zanta-Boussif, M. A. et al., 2009. Gene therapy, 16 (5), pp. 605-619). Suitably, the WPRE is a wild-type WPRE or is a mutant WPRE. For example, the WPRE may be mutated to abrogate translation of the woodchuck hepatitis virus X protein (WHX) e.g. by mutating the WHX ORF translation start codon.

In some embodiments, the WPRE comprises or consists of a nucleotide sequence which is at least 70% identical to SEQ ID NO: 79 or a fragment thereof. Suitably, the WPRE comprises or consists of a nucleotide sequence which is at least 75%, at least 80%, at least 85%, at least 90%, at least 95%, at least 96%, at least 97%, at least 98%, or at least 99% identical to SEQ ID NO: 79 or a fragment thereof.

In some embodiments, the WPRE comprises or consists of the nucleotide sequence SEQ ID NO: 79 or a fragment thereof.

Example WPRE sequence
(SEQ ID NO: 79)
aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcg

### Introns

The vector of the present invention may comprise an intron. Suitably, the nucleotide sequence encoding a TNF inhibitor is operably linked to an intron. An intron may be inserted between the promoter and nucleotide sequence encoding a TNF inhibitor to increase expression.

Suitable introns will be known to those of skill in the art (see e.g. Powell, S. K., et al., 2015. Discovery medicine, 19 (102), p. 49) and may include an MVM intron, a F.IX truncated intron 1, a chimeric β-globin/immunoglobulin heavy chain intron, a chimeric adenovirus/immunoglobulin intron, and a SV40 intron.

In some embodiments, the intron is an SV40 intron.

In some embodiments, the intron comprises or consists of a nucleotide sequence which is at least 70% identical to SEQ ID NO: 80 or a fragment thereof. Suitably, the intron comprises or consists of a nucleotide sequence which is at least 75%, at least 80%, at least 85%, at least 90%, at least 95%, at least 96%, at least 97%, at least 98%, or at least 99% identical to SEQ ID NO: 80 or a fragment thereof.

In some embodiments, the intron comprises or consists of the nucleotide sequence SEQ ID NO: 80 or a fragment thereof.

Example SV40 intron
(SEQ ID NO: 80)
gtaagtatcaaggttacaagacaggtttaaggagaccaatagaaactggg
cttgtcgagacagagaagactcttgcgtttctgataggcacctattggtc
ttactgacatccactttgcctttctctccacag

### Kozak Sequences

The vector of the present invention may comprise a Kozak sequence. Suitably, the nucleotide sequence encoding a TNF inhibitor is operably linked to a Kozak sequence. A Kozak sequence may be inserted before the start codon to improve the initiation of translation.

Suitable Kozak sequences will be known to those of skill in the art (see e.g. Kozak, M., 1987. Nucleic acids research, 15 (20), pp. 8125-8148). A consensus Kozak sequence in vertebrates may have the sequence of SEQ ID NO: 95 or SEQ ID NO: 96.

Suitably, the Kozak sequence may comprise or consist of the nucleotide sequence of SEQ ID NO: 95 or 96, or variants thereof which have five or fewer deletions, substitutions or insertions. Suitably, the variants may have four or fewer, three or fewer, two or fewer, or one deletion(s), substitution(s) or insertion(s). Suitably, the variants may have three or fewer, two or fewer, or one deletion(s) and/or three or fewer, two or fewer, or one substitution(s). Suitably, the variants may have three or fewer, two or fewer, or one deletion(s) and/or three or fewer, two or fewer, or one substitution(s). Suitably, the variants may have one deletion and/or one substitution. Suitably, the variants may have one deletion and one substitution.

| | Example consensus Kozak sequence 1 |
|---|---|
| (SEQ ID NO: 95) | |
| | gccrccatgg |
| | Example consensus Kozak sequence 2 |
| (SEQ ID NO: 96) | |
| | gccgccrccatgg |

### Inflammation-Inhibiting Oligonucleotides

The vector of the present invention may comprise a sequence encoding an inflammation-inhibiting oligonucleotide. Suitably, the nucleotide sequence encoding a TNF inhibitor is operably linked to a sequence encoding an inflammation-inhibiting oligonucleotide.

The insertion of a sequence encoding an inflammation-inhibiting oligonucleotide into a vector may reduce innate immune and T cell responses and enhanced gene expression by “cloaking” the vector from inducing unwanted immune responses. Suitable sequences will be known to those of skill in the art (see e.g. Chan, Y. K., et al., 2021. Science translational medicine, 13 (580), p.eabd3438).

The sequence encoding an inflammation-inhibiting oligonucleotide may antagonise TLR9 activation. In some embodiments, the inflammation-inhibiting oligonucleotide may be a TLR9-inhibiting oligonucleotide. In some embodiments, the sequence encoding an inflammation-inhibiting oligonucleotide includes one or more TLR9i sequences, for example one or more, two or more, three or more TLR9i sequences. Suitable TLR9i sequences are known in the art and a suitable TLR9i sequence is shown in SEQ ID NO: 81. In some embodiments, a TLR9i sequence comprises or consists of a nucleotide sequence which is at least 92%, at least 96% or 100% identical to SEQ ID NO: 81. The TLR9i sequences may be separated by any suitable linker (e.g. AAAAA linkers).

| | Example TLR9i sequence |
|---|---|
| (SEQ ID NO: 81) | |
| | tttagggttagggttagggttaggg |

In some embodiments, the sequence encoding an inflammation-inhibiting oligonucleotide comprises or consists of a nucleotide sequence which is at least 70% identical to SEQ ID NO: 82 or a fragment thereof. Suitably, the sequence encoding an inflammation-inhibiting oligonucleotide comprises or consists of a nucleotide sequence which is at least 75%, at least 80%, at least 85%, at least 90%, at least 95%, at least 96%, at least 97%, at least 98%, or at least 99% identical to SEQ ID NO: 82 or a fragment thereof.

In some embodiments, the sequence encoding an inflammation-inhibiting oligonucleotide comprises or consists of the nucleotide sequence SEQ ID NO: 82 or a fragment thereof.

Example io2 sequence
(SEQ ID NO: 82)
tttagggttagggttagggttagggaaaaatttagggttagggttagggt
tagggaaaaatttagggttagggttagggttagggaaaaatgcagcggta
agttcccatccaggttttttgcagcggtaagttcccatccaggttttttg
cagcggtaagttcccatccaggttttt

### Vectors

The vector of the present invention may be capable of transducing ocular cells (e.g. retinal ganglion cells, RPE cells, photoreceptors, glial cells). In some embodiments, the vector of the present invention is capable of specifically transducing ocular cells.

In some embodiments, the vector of the present invention is capable of transducing retinal cells. In some embodiments, the vector of the present invention is capable of specifically transducing retinal cells. The retina is the multi-layered membrane, which lines the inner posterior chamber of the eye and senses an image of the visual world which is communicated to the brain via the optic nerve. In order from the inside to the outside of the eye, the retina comprises the layers of the neurosensory retina and retinal pigment epithelium, with the choroid lying outside the retinal pigment epithelium.

In some embodiments, the vector of the present invention is capable of transducing retinal ganglion cells. In some embodiments, the vector of the present invention is capable of specifically transducing retinal ganglion cells. A retinal ganglion cell is a type of neuron located near the inner surface of the retina of the eye.

Suitable vectors for transducing ocular cells include viral vectors such as parvovirus vectors (e.g. AAV vectors), lentivirus vectors, adenovirus vectors and also non-viral delivery systems (see e.g. Rodrigues, G. A., et al., 2019. Pharmaceutical research, 36 (2), pp. 1-20).

The vector of the present invention may be a viral vector. The viral vector of the invention is preferably an adeno-associated viral (AAV), although it is contemplated that other viral vectors may be used. In some embodiments, the viral vector is any of a parvoviral vector, an adenoviral vector, a herpes simplex viral vector, an anelloviral vector, a retroviral vector, or a lentiviral vector.

The vector of the present invention may be in the form of a viral vector particle. In some embodiments, the viral vector is any of a parvoviral vector particle, an adenoviral vector particle, a herpes simplex viral vector particle, an anelloviral vector particle, a retroviral vector particle, or a lentiviral vector particle. Preferably, the viral vector of the present invention is in the form of an AAV vector particle.

Methods of preparing and modifying viral vectors and viral vector particles, such as those derived from AAV, are known in the art. Suitable methods are described in Ayuso, E., et al., 2010. Current gene therapy, 10 (6), pp. 423-436, Merten, O. W., et al., 2016. Molecular Therapy-Methods & Clinical Development, 3, p. 16017; and Nadeau, I. and Kamen, A., 2003. Biotechnology advances, 20 (7-8), pp. 475-489.

### Parvovirus Vectors

The vector of the present invention may be a parvovirus vector. The vector of the present invention may be in the form of a parvovirus vector particle.

Parvoviruses, and especially the adeno-associated virus (AAV), provide a versatile platform for the rational design of human gene-therapy vectors. Typically, all parvoviruses are composed of a small, non-enveloped capsid containing a single-stranded DNA genome. Suitably, the parvovirus vector is from the Parvovirinae subfamily, which includes Dependoparvovirus, Protoparvovirus, and Bocaparvovirus. The vector of the present invention may be a hybrid gene therapy vector based on parvoviruses (see e.g. Fakhiri, J. and Grimm, D., 2021. Molecular Therapy, 29 (12), pp. 3359-3382).

In some embodiments, the vector of the present invention is a dependoparvovirus vector. In some embodiments, the vector of the present invention is in the form of a dependoparvovirus vector. Some dependoparvoviruses are also known as adeno-associated viruses because they cannot replicate productively in their host cell without the cell being co-infected by a helper virus such as an adenovirus.

In preferred embodiments, the vector of the present invention is an adeno-associated viral (AAV) vector. In preferred embodiments, the vector of the present invention is in the form of an AAV vector particle.

### AAV Genomes

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11. In some embodiments, the AAV vector of the present invention is an AAV1, AAV2, AAV4, AAV5, AAV6, AAV7, AAV8, or AAV9 serotype, or a variant thereof. In some embodiments, the AAV vector of the present invention is an AAV2 serotype, or a variant thereof.

The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle, which determines the AAV serotype.

The AAV genome may be the full genome of a naturally occurring AAV. For example, a vector comprising a full AAV genome may be used to prepare an AAV vector or vector particle.

Preferably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11. Suitably, the AAV genome is a derivative of AAV2.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof.

Suitably, the AAV genome may comprise at least one, such as two, AAV2 ITRs, or variants thereof. In some embodiments, the AAV genome comprises an AAV2 5′ITR and/or an AAV2 3′ITR.

In some embodiments, the AAV genome comprises a 5′ITR having at least 70%, at least 75%, at least 80%, at least 85%, at least 90%, at least 95%, at least 96%, at least 97%, at least 98%, at least 99% identity, or 100% identity to SEQ ID NO: 83. In some embodiments, the AAV genome comprises a 5′ITR comprising or consisting of SEQ ID NO: 83.

Example AAV2 5′ ITR
(SEQ ID NO: 83)
ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttcct

In some embodiments, the AAV genome comprises a 3′ITR having at least 70%, at least 75%, at least 80%, at least 85%, at least 90%, at least 95%, at least 96%, at least 97%, at least 98%, at least 99% identity, or 100% identity to SEQ ID NO: 84. In some embodiments, the AAV genome comprises a 3′ITR comprising or consisting of SEQ ID NO: 84.

Example AAV2 3′ ITR
(SEQ ID NO: 84)
aggaacccctagtgatggagttggccactccctctctgcgcgctcgctcg
ctcactgaggccgggcgaccaaaggtcgcccgacgcccgggctttgcccg
ggggcctcagtgagcgagcgagcgcgcag

The one or more ITRs may flank the nucleotide sequence encoding a TNF inhibitor at either end. The inclusion of one or more ITRs is preferred to aid concatemer formation of the AAV vector in the nucleus of a host cell, for example following the conversion of single-stranded vector DNA into double-stranded DNA by the action of host cell DNA polymerases. The formation of such episomal concatamers protects the AAV vector during the life of the host cell, thereby allowing for prolonged expression of the transgene in vivo.

Suitably, the AAV genome may comprise one or more AAV2 ITR sequences flanking the nucleotide sequence encoding a TNF inhibitor. Suitably, the AAV genome may comprise two AAV2 ITR sequences flanking either side of the nucleotide sequence encoding a TNF inhibitor.

Suitably, ITR elements will be the only sequences retained from the native AAV genome in the derivative. A derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

### AAV Capsid Proteins

The AAV vector particle may be encapsidated by capsid proteins. The serotype may facilitate the transduction of ocular cells (e.g. retinal ganglion cells, RPE cells, photoreceptors, glial cells), for example specific transduction of ocular cells. The AAV vector particle may be an ocular tissue-specific vector particle. The AAV vector particle may be encapsidated by an ocular tissue-specific capsid. The AAV vector particle may comprise an ocular tissue-specific capsid protein.

In some embodiments, the AAV vector particle is a retinal-specific vector particle. In some embodiments, the AAV vector particle is encapsidated by a retinal-specific capsid. In some embodiments, the AAV vector particle comprises a retinal-specific capsid protein.

In some embodiments, the AAV vector particle is a retinal ganglion-specific vector particle. In some embodiments, the AAV vector particle is encapsidated by a retinal ganglion-specific capsid. In some embodiments, the AAV vector particle comprises a retinal ganglion-specific capsid protein.

Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of retinal cells compared to an AAV vector comprising a naturally occurring AAV genome. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of retinal cells, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

For example, a directed evolution approach has been leveraged to generate novel AAV vectors that can more effectively cross biological barriers and target specific cell types. An example is the identification of the AAV.7m8 variant which, following intravitreal injection, is capable of efficient gene delivery to all retina layers in both mice and primates. Similarly, SH10, an AAV6 variant, has increased tropism for glial cells following intravitreal delivery and has been shown to rescue retinal function in a rat model of RP (see e.g. Rodrigues, G. A., et al., 2019. Pharmaceutical research, 36 (2), pp. 1-20).

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

For example, AAV variants gave been generated by site-directed mutagenesis of surface-exposed tyrosine residues, which prevents capsid phosphorylation and subsequent ubiquitination and proteasome-mediated degradation. AAV2, AAV8, and AAV9 carrying these mutations have been shown to have increased transduction efficiency both in vitro and in vivo (see e.g. Petrs-Silva, H., et al., 2009. Molecular therapy, 17 (3), pp. 463-471).

The capsid protein may be an artificial capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned.

The capsid protein may comprise a mutation or modification relative to the wild type capsid protein which improves the ability to transduce ocular cells relative to an unmodified or wild type viral particle. Improved ability to transduce ocular cells may be measured for example by measuring the expression of a transgene, e.g. GFP, carried by the AAV vector particle, wherein expression of the transgene in ocular cells correlates with the ability of the AAV vector particle to transduce ocular cells.

Suitably, the AAV vector particle of the present invention is an AAV1, AAV2, AAV4, AAV5, AAV6, AAV7, AAV8, or AAV9 vector particle, or a variant thereof. AAV vector particles with these serotypes can transduce ocular cells.

The AAV vector particle of the present invention may comprise AAV1, AAV2, AAV4, AAV5, AAV6, AAV7, AAV8, or AAV9 capsid proteins, or variants thereof. Suitably, the AAV vector particle may comprise AAV1, AAV2, AAV4, AAV5, AAV6, AAV7, AAV8, or AAV9 capsid proteins VP1, VP2 and VP3, or variants thereof.

In one embodiment, the AAV vector particle comprises one or more AAV2 ITR sequences flanking the nucleotide sequence encoding a TNF inhibitor and AAV1, AAV2, AAV4, AAV5, AAV6, AAV7, AAV8, or AAV9 capsid proteins, or variants thereof. In one embodiment, the AAV vector particle comprises an AAV2 genome and AAV1, AAV2, AAV4, AAV5, AAV6, AAV7, AAV8, or AAV9 capsid proteins, or variants thereof.

### AAV2 Vectors and Variants Thereof

In some embodiments, the AAV vector particle is an AAV2 vector particle, or a variant thereof. In some embodiments, the AAV vector particle comprises AAV2 capsid proteins, or variants thereof. Suitably, the AAV vector particle may comprise AAV2 capsid protein VP1, VP2 and VP3, or variants thereof.

AAV2 variants include AAV2.tYF, AAV2.7m8, R100, AAV2.GL, AAV2.NN, LSV1, R195-003, and dyno-86m. In some embodiments, the AAV vector particle is an AAV2 vector particle, an AAV2.tYF vector particle, an AAV2.7m8 vector particle, a R100 vector particle, an AAV2.GL vector particle, an AAV2.NN vector particle, an LSV1 vector particle, an R195-003 vector particle, or a dyno-86m vector particle. In some embodiments, the AAV vector particle comprises AAV2 capsid proteins, AAV2.tYF capsid proteins, AAV2.7m8 capsid proteins, R100 capsid proteins, AAV2.GL capsid proteins, AAV2.NN capsid proteins, LSV1 capsid proteins, R195-003 capsid proteins, or dyno-86m capsid proteins. Suitably, the AAV vector particle may comprise AAV2 capsid protein VP1, VP2 and VP3, AAV2.tYF capsid protein VP1, VP2 and VP3, AAV2.7m8 capsid protein VP1, VP2 and VP3, R100 capsid protein VP1, VP2 and VP3, AAV2.GL capsid protein VP1, VP2 and VP3, AAV2.NN capsid protein VP1, VP2 and VP3, LSV1 capsid protein VP1, VP2 and VP3, R195-003 capsid protein VP1, VP2 and VP3, or dyno-86m capsid protein VP1, VP2 and VP3.

In some embodiments, the AAV vector particle is an AAV2 vector particle, an AAV2.tYF vector particle, an AAV2.7m8 vector particle, a R100 vector particle, an AAV2.GL vector particle, or an AAV2.NN vector particle. In some embodiments, the AAV vector particle comprises AAV2 capsid proteins, AAV2.tYF capsid proteins, AAV2.7m8 capsid proteins, R100 capsid proteins, AAV2.GL capsid proteins, or AAV2.NN capsid proteins. Suitably, the AAV vector particle may comprise AAV2 capsid protein VP1, VP2 and VP3, AAV2.tYF capsid protein VP1, VP2 and VP3, AAV2.7m8 capsid protein VP1, VP2 and VP3, R100 capsid protein VP1, VP2 and VP3, AAV2.GL capsid protein VP1, VP2 and VP3, or AAV2.NN capsid protein VP1, VP2 and VP3.

In some embodiments, the AAV vector particle is an AAV2 vector particle. In some embodiments, the AAV vector particle comprises AAV2 capsid proteins. In some embodiments, the AAV vector particle comprises AAV2 capsid protein VP1, VP2 and VP3.

Suitably, an AAV2 VP1 capsid protein may comprise or consist of the amino acid sequence SEQ ID NO: 85, or a variant which is at least 90% identical to SEQ ID NO: 85. Suitably, the variant may be at least 95%, at least 96%, at least 97%, at least 98% or at least 99% identical to SEQ ID NO: 85. Suitably, an AAV2 VP2 and VP3 capsid protein may be an N-terminal truncation of SEQ ID NO: 85, or an N-terminal truncation of a variant which is at least 90% identical, at least 95%, at least 96%, at least 97%, at least 98% or at least 99% identical to SEQ ID NO: 85.

Example AAV2 VP1 capsid protein
(SEQ ID NO: 85)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSFGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHFSPRDWQRLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTESYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTESAAKFASFITQYSTGQVSVEIEWELQKENSKRWNPEIQY
TSNYNKSVNVDFTVDINGVYSEPRPIGTRYLTRNL

In some embodiments, the AAV vector particle is an AAV2.tYF vector particle. In some embodiments, the AAV vector particle comprises AAV2.tYF capsid proteins. In some embodiments, the AAV vector particle comprises AAV2.tYF capsid protein VP1, VP2 and VP3. Single phenylalanine (F) for tyrosine (Y) substitutions had increased the potency of AAV2 following intraocular injection (see e.g. Petrs-Silva, H., et al., 2009. Molecular therapy, 17 (3), pp. 463-471).

Suitably, an AAV2.tYF VP1 capsid protein may comprise or consist of the amino acid sequence SEQ ID NO: 86, or a variant which is at least 90% identical to SEQ ID NO: 86. Suitably, the variant may be at least 95%, at least 96%, at least 97%, at least 98% or at least 99% identical to SEQ ID NO: 86. Suitably, an AAV2.tYF VP2 and VP3 capsid protein may be an N-terminal truncation of SEQ ID NO: 86, or an N-terminal truncation of a variant which is at least 90% identical, at least 95%, at least 96%, at least 97%, at least 98% or at least 99% identical to SEQ ID NO: 86.

Example AAV2. tYF VP1 capsid protein
(SEQ ID NO: 86)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSEGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHESPRDWORLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTESYTFEDVPFHSSYAHSQSLDRLMNPLIDQYLYFLSRTNT
PSGTTTQSRLQFSQAGASDIRDQSRNWLPGPCYRQQRVSKTSADNNNSEF
SWTGATKYHLNGRDSLVNPGPAMASHKDDEEKFFPQSGVLIFGKQGSEKT
NVDIEKVMITDEEEIRTTNPVATEQYGSVSTNLORGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDINGVYSEPRPIGTRFLTRNL

In some embodiments, the AAV vector particle is an AAV2.7m8 vector particle. In some embodiments, the AAV vector particle comprises AAV2.7m8 capsid proteins. In some embodiments, the AAV vector particle comprises AAV2.7m8 capsid protein VP1, VP2 and VP3. AAV2.7m8 is an engineered capsid with a 10-amino acid insertion in adeno-associated virus (AAV) surface variable region VIII (VR-VIII) resulting in the ability to efficiently transduce retina cells following intravitreal administration (see e.g. Bennett, A., et al., 2020. Journal of structural biology, 209 (2), p. 107433).

Suitably, an AAV2.7m8 VP1 capsid protein may comprise or consist of the amino acid sequence SEQ ID NO: 87, or a variant which is at least 90% identical to SEQ ID NO: 87. Suitably, the variant may be at least 95%, at least 96%, at least 97%, at least 98% or at least 99% identical to SEQ ID NO: 87. Suitably, an AAV2.7m8 VP2 and VP3 capsid protein may be an N-terminal truncation of SEQ ID NO: 87, or an N-terminal truncation of a variant which is at least 90% identical, at least 95%, at least 96%, at least 97%, at least 98% or at least 99% identical to SEQ ID NO: 87.

Example AAV2. 7m8 VP1 capsid protein
(SEQ ID NO: 87)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSEGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHFSPRDWQRLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLORGNLALGETTRPARQA
ATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEN
SKRWNPEIQYTSNYNKSVNVDFTVDINGVYSEPRPIGTRYLTRNL

In some embodiments, the AAV vector particle is a R100 vector particle. In some embodiments, the AAV vector particle comprises R100 capsid proteins. In some embodiments, the AAV vector particle comprises R100 capsid protein VP1, VP2 and VP3. R100 demonstrated superior transduction of human retinal cells compared to wildtype AAV (see e.g. Kotterman, M., et al., 2021. bioRxiv 2021.06.24.449775).

Suitably, a R100 VP1 capsid protein may comprise or consist of the amino acid sequence SEQ ID NO: 88, or a variant which is at least 90% identical to SEQ ID NO: 88. Suitably, the variant may be at least 95%, at least 96%, at least 97%, at least 98% or at least 99% identical to SEQ ID NO: 88. Suitably, a R100 and VP3 capsid protein may be an N-terminal truncation of SEQ ID NO: 88, or an N-terminal truncation of a variant which is at least 90% identical, at least 95%, at least 96%, at least 97%, at least 98% or at least 99% identical to SEQ ID NO: 88.

Example R100 VP1 capsid protein
(SEQ ID NO: 88)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSEGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHESPRDWQRLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNLAISDQTKHARQA
ATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEN
SKRWNPEIQYTSNYNKSVNVDFTVDINGVYSEPRPIGTRYLTRNL

In some embodiments, the AAV vector particle is an AAV2.GL vector particle. In some embodiments, the AAV vector particle comprises AAV2.GL capsid proteins. In some embodiments, the AAV vector particle comprises AAV2.GL capsid protein VP1, VP2 and VP3. In some embodiments, the AAV vector particle is an AAV2.NN vector particle. In some embodiments, the AAV vector particle comprises AAV2.NN capsid proteins. In some embodiments, the AAV vector particle comprises AAV2.NN capsid protein VP1, VP2 and VP3. AAV2.GL and AAV2.NN mediate widespread and high-level retinal transduction after intravitreal injection in mice, dogs and non-human primates (see e.g. Pavlou, M., et al., 2021. EMBO molecular medicine, 13 (4), p.e13392).

Suitably, an AAV2.GL VP1 capsid protein may comprise or consist of the amino acid sequence SEQ ID NO: 89, or a variant which is at least 90% identical to SEQ ID NO: 89. Suitably, the variant may be at least 95%, at least 96%, at least 97%, at least 98% or at least 99% identical to SEQ ID NO: 89. Suitably, an AAV2.GL VP2 and VP3 capsid protein may be an N-terminal truncation of SEQ ID NO: 89, or an N-terminal truncation of a variant which is at least 90% identical, at least 95%, at least 96%, at least 97%, at least 98% or at least 99% identical to SEQ ID NO: 89.

Example AAV2. GL VP1 capsid protein
(SEQ ID NO: 89)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSFGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHESPRDWQRLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIEGKQGSEKT
NVDIEKVMITDEEEIRTTNPVATEQYGSVSTNLQRGNAAAGLSPPTRAAR
QAATADVNTQGVLPGMVWQDRDVYLQGPIWAKIPHTDGHFHPSPLMGGFG
LKHPPPQILIKNTPVPANPSTTFSAAKFASFITQYSTGQVSVEIEWELQK
ENSKRWNPEIQYTSNYNKSVNVDFTVDINGVYSEPRPIGTRYLTRNL

Suitably, an AAV2.NN VP1 capsid protein may comprise or consist of the amino acid sequence SEQ ID NO: 90, or a variant which is at least 90% identical to SEQ ID NO: 90. Suitably, the variant may be at least 95%, at least 96%, at least 97%, at least 98% or at least 99% identical to SEQ ID NO: 90. Suitably, an AAV2.NN VP2 and VP3 capsid protein may be an N-terminal truncation of SEQ ID NO: 90, or an N-terminal truncation of a variant which is at least 90% identical, at least 95%, at least 96%, at least 97%, at least 98% or at least 99% identical to SEQ ID NO: 90.

Example AAV2.NN VP1 capsid protein
(SEQ ID NO: 90)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSEGGNLGRAVEQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDENRFHCHESPRDWQRLI
NNNWGFRPKRLNFKLFNIQVKEVTQNDGTTTIANNLTSTVQVFTDSEYQL
PYVLGSAHQGCLPPFPADVEMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNAAANNPTPSRAAR
QAATADVNTQGVLPGMVWQDRDVYLQGPIWAKIPHTDGHFHPSPLMGGFG
LKHPPPQILIKNTPVPANPSTTFSAAKFASFITQYSTGQVSVEIEWELQK
ENSKRWNPEIQYTSNYNKSVNVDFTVDINGVYSEPRPIGTRYLTRNL

In some embodiments, the AAV vector particle is a LSV1 vector particle. In some embodiments, the AAV vector particle comprises LSV1 capsid proteins. In some embodiments, the AAV vector particle comprises LSV1 capsid protein VP1, VP2 and VP3. Loop swap variant 1 (LSV1) transduces the retina and retinal pigment epithelium (RPE) from the vitreous and is based on AAV2.5T, a substitution from aa 571-579 (see e.g. Baker, C. K., et al., 2022, Molecular Therapy, 30 (4), p. 575).

Suitably, a LSV1 VP1 capsid protein may comprise or consist of the amino acid sequence SEQ ID NO: 94 or a variant which is at least 90% identical to SEQ ID NO: 94 Suitably, the variant may be at least 95%, at least 96%, at least 97%, at least 98% or at least 99% identical to SEQ ID NO: 94 Suitably, a LSV1 VP2 and VP3 capsid protein may be an N-terminal truncation of SEQ ID NO: 94 or an N-terminal truncation of a variant which is at least 90% identical, at least 95%, at least 96%, at least 97%, at least 98% or at least 99% identical to SEQ ID NO: 94

Example LSV1 capsid protein
(SEQ ID NO: 94)
MAADGYLPDWLEDTLSEGIRQWWKLKPGPPPPKPAERHKDDSRGLVLPGY
KYLGPENGLDKGEPVNEADAAALEHDKAYDRQLDSGDNPYLKYNHADAEF
QERLKEDTSEGGNLGRAVEQAKKRVLEPFGLVEEGAKTAPTGKRIDDHFP
KRKKARTEEDSKPSTSSDAEAGPSGSQQLQIPAQPASSLGADTMSAGGGG
PLGDNNQGADGVGNASGDWHCDSTWMGDRVVTKSTRTWVLPSYNNHQYRE
IKSGSVDGSNANAYFGYSTPWGYFDFNRFHSHWSPRDWORLINNYWGFRP
RSLRVKIFNIQVKEVTVQDSTTTIANNLTSTVQVFTDDDYQLPYVVGNGT
EGCLPAFPPQVFTLPQYGYATLNRDNTENPTERSSFFCLEYFPSKMLRTG
NNFEFTYNFEEVPFHSSFAPSQNLEKLANPLVDQYLYRFVSTNNTGGVQF
NKNLAGRYANTYKNWFPGPMGRTQGWNLGSGVNRASVSAFATTNRMELEG
ASYQVPPQPNGMTNNLQGSNTYALENTMIENSQPANPGTTATYLEGNMLI
TSESETQPVNRVAYNVGGQMLAHKFKSGDAPTTGTYNLQEIVPGSVWMER
DVYLQGPIWAKIPETGAHFHPSPAMGGFGLKHPPPMMLIKNTPVPGNITS
FSDVPVSSFITQYSTGQVTVEMEWELKKENSKRWNPEIQYTNNYNDPQFV
DFAPDSTGEYRTTRPIGTRYLTRPL

In some embodiments, the AAV vector particle is a R195-003 vector particle. In some embodiments, the AAV vector particle comprises R195-003 capsid proteins. In some embodiments, the AAV vector particle comprises R195-003 capsid protein VP1, VP2 and VP3 (see e.g. Human Gene Therapy Methods 2022; 33 (23-24): A27-A28).

In some embodiments, the AAV vector particle is a dyno-86m vector particle. In some embodiments, the AAV vector particle comprises dyno-86m capsid proteins. In some embodiments, the AAV vector particle comprises dyno-86m capsid protein VP1, VP2 and VP3 (see e.g. Molecular Therapy 2023; 31 (4), S1, p. 1284).

In some embodiments, the AAV vector particle comprises one or more AAV2 ITR sequences flanking the nucleotide sequence encoding a TNF inhibitor and AAV2 capsid proteins, or variants thereof. In some embodiments, the AAV vector particle comprises an AAV2 genome and AAV2 capsid proteins, or variants thereof.

### Other Parvovirus Vectors

In some embodiments, the vector of the present invention is a protoparvovirus vector. In some embodiments, the vector of the present invention is in the form of a protoparvovirus vector. Protoparvoviruses have been studied extensively and utilized as vectors, including the minute virus of mice (MVM), the rat parvovirus H1, and the LuIII virus. Human variants that have been found recently include bufavirus (BuV), tusavirus (TuV), and cutavirus (CuV) (see e.g. Fakhiri, J. and Grimm, D., 2021. Molecular Therapy, 29 (12), pp. 3359-3382).

In some embodiments, the vector of the present invention is a bocaparvovirus vector. In some embodiments, the vector of the present invention is in the form of a bocaparvovirus vector. The use of Human bocavirus 1 (HBoV1) as a parvoviral vector for gene delivery has been described (see e.g. Shao, L., et al., 2021. Frontiers in Microbiology, 12, p. 1463).

### Other Viral Vectors

### Retroviral and Lentiviral Vectors

The vector of the present invention may be a retroviral vector or a lentiviral vector. The vector of the present invention may be a retroviral vector particle or a lentiviral vector particle.

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV).

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells. In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Preferably, the viral vector used in the present invention has a minimal viral genome. By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilization by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site) or by modifying or deleting essential att sequences from the vector LTR, or by a combination of the above.

### Adenoviral Vectors

The vector of the present invention may be an adenoviral vector. The vector of the present invention may be an adenoviral vector particle.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Herpes Simplex Viral Vector

The vector of the present invention may be a herpes simplex viral vector. The vector of the present invention may be a herpes simplex viral vector particle.

Herpes simplex virus (HSV) is a neurotropic DNA virus with favorable properties as a gene delivery vector. HSV is highly infectious, so HSV vectors are efficient vehicles for the delivery of exogenous genetic material to cells. Viral replication is readily disrupted by null mutations in immediate early genes that in vitro can be complemented in trans, enabling straightforward production of high-titre pure preparations of non-pathogenic vector. The genome is large (152 Kb) and many of the viral genes are dispensable for replication in vitro, allowing their replacement with large or multiple transgenes. Latent infection with wild-type virus results in episomal viral persistence in sensory neuronal nuclei for the duration of the host lifetime. The vectors are non-pathogenic, unable to reactivate and persist long-term. The latency active promoter complex can be exploited in vector design to achieve long-term stable transgene expression in the nervous system. HSV vectors transduce a broad range of tissues because of the wide expression pattern of the cellular receptors recognized by the virus. Increasing understanding of the processes involved in cellular entry has allowed targeting the tropism of HSV vectors.

### Vaccinia Virus Vectors

The vector of the present invention may be a vaccinia viral vector. The vector of the present invention may be a vaccinia viral vector particle.

Vaccinia virus is large enveloped virus that has an approximately 190 kb linear, double-stranded DNA genome. Vaccinia virus can accommodate up to approximately 25 kb of foreign DNA, which also makes it useful for the delivery of large genes. A number of attenuated vaccinia virus strains are known in the art that are suitable for gene therapy applications, for example the MVA and NYVAC strains.

### Anellovirus Vectors

The vector of the present invention may be an anelloviral vector. The vector of the present invention may be an anelloviral vector particle.

Anelloviruses are small, single stranded circular DNA viruses. They are extremely diverse and have not been associated with any disease so far. Anelloviruses may infect the complete human population, and there is no evidence of disease association or of viral clearance from infected individuals (see e.g. Venkataraman, T., et al., 2022. bioRxiv 2022.03.28.486145).

### Non-Viral Delivery Systems

In some embodiments, the vector is a non-viral vector. Suitable non-viral delivery systems will be known to the skilled person (see e.g. Zulliger, R., et al., 2015. Journal of Controlled Release, 219, pp. 471-487; and Oliveira, A. V., et al., 2017. Materials Science and Engineering: C, 77, pp. 1275-1289).

In some embodiments, the vector is a plasmid. In some embodiments, the plasmid is modified to facilitate cellular and/or nuclear uptake. In some embodiments, the plasmid is comprised in a non-viral particle, e.g. a lipoplex particle or a polyplex particle.

Non-viral delivery systems include but are not limited to transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs), and combinations thereof.

### Example Vectors

The vector of the present invention may comprise from 5′ to 3′: a promoter (e.g. an inflammation-inducible promoter) and a nucleotide sequence encoding a TNF inhibitor.

The vector of the present invention may further comprise any other suitable elements, such as any other elements described herein or one or more spacer sequence. The spacer sequence(s) may comprise, for example, at least one (e.g. 1, 2, 3, 4, 5, 6, 7, 8, 9, 10), at least two, at least three, at least four, at least five, at least six, at least seven, at least eight, at least nine, or at least ten nucleotide bases. A spacer sequence may comprise a restriction site to enable the insertion of one or more further elements.

In preferred embodiments, the vector of the present invention is an AAV vector. In some embodiments, the AAV genome comprises from 5′ to 3′: a 5′ITR; an inflammation inducible promoter; a nucleotide sequence encoding a TNF inhibitor; and a 3′ITR. In some embodiments, the AAV genome comprises from 5′ to 3′: a 5′ITR; an inflammation inducible promoter; a nucleotide sequence encoding a TNF inhibitor; a polyadenylation sequence; and a 3′ITR. In some embodiments, the AAV genome comprises from 5′ to 3′: a 5′ITR; an inflammation inducible promoter; a nucleotide sequence encoding a TNF inhibitor; a WPRE; a polyadenylation sequence and a 3′ITR. In some embodiments, the AAV genome comprises from 5′ to 3′: a 5′ITR; an inflammation inducible promoter; an intron; a nucleotide sequence encoding a TNF inhibitor; a WPRE; a polyadenylation sequence; and a 3′ITR. In some embodiments, the AAV genome comprises from 5′ to 3′: a 5′ITR; an inflammation inducible promoter; an intron; a nucleotide sequence encoding a TNF inhibitor; a WPRE; a polyadenylation sequence; a sequence encoding an inflammation-inhibiting oligonucleotide; and a 3′ITR.

In one aspect, the present invention provides a vector comprising or consisting of a nucleotide sequence which is at least 70% identical to SEQ ID NO: 91 or a fragment thereof. Suitably, the vector comprises or consists of a nucleotide sequence which is at least 75%, at least 80%, at least 85%, at least 90%, at least 95%, at least 96%, at least 97%, at least 98%, or at least 99% identical to SEQ ID NO: 91 or a fragment thereof. In some embodiments, the vector comprises or consists of the nucleotide sequence SEQ ID NO: 91 or a fragment thereof.

In some embodiments, the AAV genome comprises or consists of a nucleotide sequence which is at least 70% identical to SEQ ID NO: 91 or a fragment thereof. Suitably, the AAV genome comprises or consists of a nucleotide sequence which is at least 75%, at least 80%, at least 85%, at least 90%, at least 95%, at least 96%, at least 97%, at least 98%, or at least 99% identical to SEQ ID NO: 91 or a fragment thereof. In some embodiments, the AAV genome comprises or consists of the nucleotide sequence SEQ ID NO: 91 or a fragment thereof.

Example AAV vector encoding adalimumab Fab
(SEQ ID NO: 91)
ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttccttgtagttaatgattaacccg
ccatgctacttatctacgtagccatgctctaggaagatcggaattcgccc
ttaagctagctgagtcactgactcagtgagtcactgactcagtgagtaag
gaattctggggactttccactggggactttccactggggactttccactg
gggactttccactggggactttccactcctgcaggagcttgaataaaatg
aatattagaagctgttagaataagagaaaatgacagaggaaaactgaaag
ggagaactgaaagtgggaaattcctctgaggcagaaaggaccatccctta
taaatagcacaggccatgaaggaagatcattctcactgcagcctttgaca
gcctttgcctcatcttgcagatcctgcagaagttggtcgtgaggcactgg
gcaggtaagtatcaaggttacaagacaggtttaaggagaccaatagaaac
tgggcttgtcgagacagagaagactcttgcgtttctgataggcacctatt
ggtcttactgacatccactttgcctttctctccacaggtgtccaggcggc
cgccatggccacaggctctcggaccagcctgctgctggccttcggcctgc
tgtgtctgccttggctgcaagagggcagcgccgaggtgcaactggtggaa
agcggcggcggcctggtccagcctggaaggtccctgagactgagctgtgc
cgccagcggctttaccttcgacgactacgccatgcactgggtgcgccagg
cccctggcaagggcctggaatgggtctccgccatcacctggaatagcggc
cacatcgactacgccgatagcgtggaaggcagattcaccatcagccggga
caacgccaagaactctctgtatctgcaaatgaacagcctgcgggctgaag
atacagccgtgtactattgcgccaaagtgagctacctctccaccgccagc
agcctggactattggggacagggcaccctggtgaccgtgtctagcgcctc
cacaaagggcccttctgtgtttccactggctccaagctccaaaagcacat
ctggaggaaccgctgccctgggctgcctggttaaggactacttccccgag
cctgtgaccgtgagctggaacagcggcgccctgacatctggtgttcatac
cttccctgccgttctgcaatcttctggactctacagcctgtcttctgtgg
tgaccgtgcccagcagcagccttggaacacagacctacatctgcaatgtg
aaccacaagcctagcaacaccaaggtggacaagaaggtgagaaagcggag
aagcggaagcggtgctaccaacttcagcctcctgaaacaggccggcgatg
tggaggaaaaccctggacctatggctaccggcagcagaaccagcctgctg
ctggcattcggccttctgtgcctgccttggctgcaagagggctctgccga
catccagatgacccagagcccttcctcactgagcgccagcgtgggcgaca
gagtgactattacatgcagagccagccaaggcatccggaactacctggcc
tggtatcagcagaagcccggcaaagcccctaagctgctgatctacgccgc
cagcaccctgcaaagcggcgtgcctagcagattcagcggctcaggctctg
gcactgatttcaccctgaccatctcctctctgcaacctgaggacgtggcc
acatactactgccagagatacaacagagccccatacacctttggccaggg
cacaaaagtggaaatcaagagaaccgtggccgctcccagtgtgttcatct
tcccccccagtgatgagcagctgaagtccggcacagcctctgtcgtgtgc
ctgctgaacaacttctaccccagagaggccaaggtgcagtggaaggtgga
taatgccctgcaaagcggcaacagccaggagagcgtgacagagcaggaca
gcaaggacagcacctacagcctctctagcacactgaccctgagcaaggcc
gactacgagaagcacaaggtgtacgcatgcgaggtgacccaccagggcct
gagcagtcctgtgaccaagagcttcaaccggggcgagtgttacccatacg
atgttccagattacgcttgataagcttggatccaatcaacctctggatta
caaaatttgtgaaagattgactggtattcttaactatgttgctcctttta
cgctatgtggatacgctgctttaatgcctttgtatcatgctattgcttcc
cgtatggctttcattttctcctccttgtataaatcctggttgctgtctct
ttatgaggagttgtggcccgttgtcaggcaacgtggcgtggtgtgcactg
tgtttgctgacgcaacccccactggttggggcattgccaccacctgtcag
ctcctttccgggactttcgctttccccctccctattgccacggcggaact
catcgccgcctgccttgcccgctgctggacaggggctcggctgttgggca
ctgacaattccgtggtgttgtcggggaaatcatcgtcctttccttggctg
ctcgcctgtgttgccacctggattctgcgcgggacgtccttctgctacgt
cccttcggccctcaatccagcggaccttccttcccgcggcctgctgccgg
ctctgcggcctcttccgcgtcttcgagatctgcctcgactgtgccttcta
gttgccagccatctgttgtttgcccctcccccgtgccttccttgaccctg
gaaggtgccactcccactgtcctttcctaataaaatgaggaaattgcatc
gcattgtctgagtaggtgtcattctattctggggggtggggtggggcagg
acagcaagggggaggattgggaagacaatagcaggcatgctggggaggta
cctttagggttagggttagggttagggaaaaatttagggttagggttagg
gttagggaaaaatttagggttagggttagggttagggaaaaatgcagcgg
taagttcccatccaggttttttgcagcggtaagttcccatccaggttttt
tgcagcggtaagttcccatccaggtttttctcgagttaagggcgaattcc
cgataaggatcttcctagagcatggctacgtagataagtagcatggcggg
ttaatcattaactacaaggaacccctagtgatggagttggccactccctc
tctgcgcgctcgctcgctcactgaggccgggcgaccaaaggtcgcccgac
gcccgggctttgcccgggcggcctcagtgagcgagcgagcgcgcag

In one aspect, the present invention provides a vector comprising or consisting of a nucleotide sequence which is at least 70% identical to SEQ ID NO: 92 or a fragment thereof. Suitably, the vector comprises or consists of a nucleotide sequence which is at least 75%, at least 80%, at least 85%, at least 90%, at least 95%, at least 96%, at least 97%, at least 98%, or at least 99% identical to SEQ ID NO: 92 or a fragment thereof. In some embodiments, the vector comprises or consists of the nucleotide sequence SEQ ID NO: 92 or a fragment thereof.

In some embodiments, the AAV genome comprises or consists of a nucleotide sequence which is at least 70% identical to SEQ ID NO: 92 or a fragment thereof. Suitably, the AAV genome comprises or consists of a nucleotide sequence which is at least 75%, at least 80%, at least 85%, at least 90%, at least 95%, at least 96%, at least 97%, at least 98%, or at least AAV genome % identical to SEQ ID NO: 92 or a fragment thereof. In some embodiments, the vector comprises or consists of the nucleotide sequence SEQ ID NO: 92 or a fragment thereof.

Example AAV vector encoding infliximab Fab
(SEQ ID NO: 92)
ctgcgcgctcgctcgctcactgaggccgcccgggcaaagcccgggcgtcg
ggcgacctttggtcgcccggcctcagtgagcgagcgagcgcgcagagagg
gagtggccaactccatcactaggggttccttgtagttaatgattaacccg
ccatgctacttatctacgtagccatgctctaggaagatcggaattcgccc
ttaagctagctgagtcactgactcagtgagtcactgactcagtgagtaag
gaattctggggactttccactggggactttccactggggactttccactg
gggactttccactggggactttccactcctgcaggagcttgaataaaatg
aatattagaagctgttagaataagagaaaatgacagaggaaaactgaaag
ggagaactgaaagtgggaaattcctctgaggcagaaaggaccatccctta
taaatagcacaggccatgaaggaagatcattctcactgcagcctttgaca
gcctttgcctcatcttgcagatcctgcagaagttggtcgtgaggcactgg
gcaggtaagtatcaaggttacaagacaggtttaaggagaccaatagaaac
tgggcttgtcgagacagagaagactcttgcgtttctgataggcacctatt
ggtcttactgacatccactttgcctttctctccacaggtgtccaggcggc
cgccatggcaacaggtagtcgaaccagcctattactggccttcggtctcc
tgtgtctgccctggcttcaagagggctctgctgaggtgaaactggaggaa
agtggaggaggcctcgttcaaccaggcggctccatgaagctgtcgtgtgt
ggcatctggcttcatcttcagcaatcactggatgaactgggtcaggcaat
ctcctgagaaggggctagagtgggtggcggagatccgctcaaaatcaatc
aattccgccacacattatgcagagtcagtaaaagggcggttcaccatttc
tagagatgacagcaaaagcgccgtgtacctccagatgaccgacctgcgaa
cagaggacactggggtctactactgctcccggaactactatggctccacc
tatgactactggggccaagggaccacattgacagtatcctcagcctccac
taaaggtccttcagtgtttccgctggctccctcctccaaaagtacgtcag
gcggcaccgctgctctgggctgtctggtgaaggattacttccctgaacct
gtgactgtttcctggaacagtggagccttgacttcaggagtccacacatt
tccggcagtgctccagagcagtggtctctattccctaagcagtgtagtga
ccgtgccctctagcagcctcggaacccagacatacatctgcaatgtcaat
cacaagccaagcaatacaaaagtggacaagaaggttaggaagcggcgttc
cgggagcggggctacgaatttttcattgctcaagcaagcgggagatgtgg
aggagaaccctggccccatggccacgggaagtcggacttccttactactc
gcctttggtcttctttgcttgccatggctccaggagggtagtgcagacat
tctcctgacccagtcccctgctatcttgtctgtctcccccggagagcgcg
tctccttctcttgcagagcttcccagtttgtgggcagcagcattcactgg
tatcagcagagaacaaatggatcaccaaggcttttgatcaagtatgcttc
agaaagcatgagtgggataccatccaggtttagtggaagtggctctggta
ctgacttcactctctctataaacacggtggaaagcgaagatattgctgac
tattactgtcagcaaagccatagctggccatttacttttggatcagggac
caacctggaagtcaagagaactgtggccgcgccttcggtttttattttcc
ccccatctgatgaacagctgaagagcggtacagccagtgtagtgtgcctg
ctcaacaacttctaccctagagaagccaaggtgcagtggaaggtcgacaa
tgcattacagagcgggaacagccaggaaagtgttactgagcaggatagca
aggacagcacctactctctgtctagcacactcactttgtctaaagcagat
tatgagaaacataaagtttatgcctgtgaagttacccaccagggcctgag
cagtcccgtcaccaagtctttcaaccgcggggagtgctacccatacgatg
ttccagattacgcttgataagcttggatccaatcaacctctggattacaa
aatttgtgaaagattgactggtattcttaactatgttgctccttttacgc
tatgtggatacgctgctttaatgcctttgtatcatgctattgcttcccgt
atggctttcattttctcctccttgtataaatcctggttgctgtctcttta
tgaggagttgtggcccgttgtcaggcaacgtggcgtggtgtgcactgtgt
ttgctgacgcaacccccactggttggggcattgccaccacctgtcagctc
ctttccgggactttcgctttccccctccctattgccacggcggaactcat
cgccgcctgccttgcccgctgctggacaggggctcggctgttgggcactg
acaattccgtggtgttgtcggggaaatcatcgtcctttccttggctgctc
gcctgtgttgccacctggattctgcgcgggacgtccttctgctacgtccc
ttcggccctcaatccagcggaccttccttcccgcggcctgctgccggctc
tgcggcctcttccgcgtcttcgagatctgcctcgactgtgccttctagtt
gccagccatctgttgtttgcccctcccccgtgccttccttgaccctggaa
ggtgccactcccactgtcctttcctaataaaatgaggaaattgcatcgca
ttgtctgagtaggtgtcattctattctggggggtggggtggggcaggaca
gcaagggggaggattgggaagacaatagcaggcatgctggggaggtacct
ttagggttagggttagggttagggaaaaatttagggttagggttagggtt
agggaaaaatttagggttagggttagggttagggaaaaatgcagcggtaa
gttcccatccaggttttttgcagcggtaagttcccatccaggttttttgc
agcggtaagttcccatccaggtttttctcgagttaagggcgaattcccga
taaggatcttcctagagcatggctacgtagataagtagcatggcgggtta
atcattaactacaaggaacccctagtgatggagttggccactccctctct
gcgcgctcgctcgctcactgaggccgggcgaccaaaggtcgcccgacgcc
cgggctttgcccgggcggcctcagtgagcgagcgagcgcgcag

### Variants, Derivatives, Analogues, and Fragments

In addition to the specific polypeptides and polynucleotides mentioned herein, the invention also encompasses variants, derivatives, and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one or all of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one or all of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Polypeptides used in the invention may also have deletions, insertions or substitutions of amino acid residues which produce a silent change and result in a functionally equivalent polypeptide. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The effect of additions, deletions, substitutions, modifications, replacements and/or variations may be predicted using any suitable prediction tool e.g. SIFT (Vaser, R., et al., 2016. Nature protocols, 11 (1), pp. 1-9), PolyPhen-2 (Adzhubei, I., et al., 2013. Current protocols in human genetics, 76 (1), pp. 7-20), CADD (Rentzsch, P., et al., 2021. Genome medicine, 13 (1), pp. 1-12), REVEL (Ioannidis, N. M., et al., 2016. The American Journal of Human Genetics, 99 (4), pp. 877-885), MetaLR (Dong, C., et al., 2015. Human molecular genetics, 24 (8), pp. 2125-2137), and/or MutationAssessor (Reva, B., et al., 2011. Nucleic acids research, 39 (17), pp.e118-e118) or based on clinical data e.g. ClinVar (Landrum, M. J., et al., 2016. Nucleic acids research, 44 (D1), pp.D862-D868). Suitable additions, deletions, substitutions, modifications, replacements and/or variations may be considered tolerated, benign, and/or likely benign.

Typically, a variant may have a certain identity with the wild type amino acid sequence or the wild type nucleotide sequence.

In the present context, a variant sequence is taken to include an amino acid sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, at least 55%, at least 60%, at least 65%, at least 70%, at least 75%, at least 80%, at least 85% or at least 90% identical, suitably at least 95%, at least 96%, at least 97%, at least 98% or at least 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (see e.g. Devereux, J., et al., 1984. Nucleic acids research, 12 (1), pp. 387-395). Examples of other software that can perform sequence comparisons include, but are not limited to, the BLAST package (see e.g. Altschul, S. F., et al., 1990. Journal of molecular biology, 215 (3), pp. 403-410), BLAST 2 (see e.g. Tatusova, T. A. and Madden, T. L., 1999. FEMS microbiology letters, 174 (2), pp. 247-250), FASTA (see e.g. Pearson, W. R. and Lipman, D. J., 1988. PNAS, 85 (8), pp. 2444-2448), EMBOSS Needle (Madeira, F., et al., 2019. Nucleic acids research, 47 (W1), pp.W636-W641) and the GENEWORKS suite of comparison tools. For some applications, it is preferred to use EMBOSS Needle.

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives, and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded polypeptide. These methods are only illustrative of the numerous standard techniques known in the art for manipulation of DNA sequences and other known techniques may also be used.

### Vectors, Kits, and Systems

In one aspect, wherein the vector is a viral vector, the present invention provides a vector encoding the viral genome of the present invention. The vector may be a transfer vector, as described herein. For example, the vector may be a plasmid and/or the viral genome may be operably linked to a promoter (e.g. a viral promoter, such as a CMV promoter).

In one aspect, the present invention provides a kit or system for producing the vector (e.g. viral vector) of the present invention. The kit or system may be a virus packaging kit or system or a virus production kit or system. As used herein, a “virus packaging kit or system” may comprise one or more components, and optionally instructions, for packaging the viral vector of the present invention. As used herein, a “virus production kit or system” may comprise one or more components, and optionally instructions, for producing the viral vector of the present invention.

The kit or system may comprise a transfer vector encoding the viral genome of the present invention and optionally one or more helper vectors. The kit or system may further comprise host cells (e.g. packaging cells or producer cells) and/or other reagents (e.g. transfection reagent, culture medium, etc.). The kit or system may further comprise any other suitable components, and optionally instructions for packaging and/or producing the viral vector of the present invention.

### Cells

In one aspect, the present invention provides a cell comprising the vector (e.g. viral vector) of the present invention. The cell may be an isolated cell. Suitably, the cell is a mammalian cell, for example a human cell. The cell may be an isolated human cell.

Suitably, the cell may be a producer cell. The term “producer cell” includes a cell that produces viral particles, after transient transfection, stable transfection or vector transduction of all the elements necessary to produce the viral particles or any cell engineered to stably comprise the elements necessary to produce the viral particles. In some embodiments, the producer cell is an AAV producer cell. Suitable producer cells will be known to those of skill in the art (see e.g. Martin, J., et al. 2013. Human gene therapy methods, 24 (4), pp. 253-269) and may include HEK293, COS-1, COS-7, CV-1, HeLa, CHO, and A549 cell lines. In some embodiments, the producer cell is a HEK293 cell, or a derivative thereof (e.g. a HEK293T cell).

Suitably, the cell may be a packaging cell. The term “packaging cell” includes a cell which contains some or all of the elements necessary for packaging a recombinant virus genome. Typically, such packaging cells contain one or more vectors which are capable of expressing viral structural proteins (e.g. AAV rep and cap genes) and/or one or more genes encoding the viral structural proteins have been integrated into the genome of the packaging cell. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell”. In some embodiments, the packaging cell is an AAV packaging cell. Suitable packaging cells will be known to those of skill in the art (see e.g. Martin, J., et al. 2013. Human gene therapy methods, 24 (4), pp. 253-269).

### Pharmaceutical Composition

In one aspect, the present invention provides a pharmaceutical composition comprising the vector or cell of the present invention. In preferred embodiments, the pharmaceutical composition comprises the vector of the present invention in the form of a viral vector particle.

A pharmaceutical composition is a composition that comprises or consists of a therapeutically effective amount of a pharmaceutically active agent e.g. the vector. A pharmaceutical composition preferably includes a pharmaceutically acceptable carrier, diluent or excipient (including combinations thereof).

By “pharmaceutically acceptable” is included that the formulation is sterile and pyrogen free. The carrier, diluent, and/or excipient must be “acceptable” in the sense of being compatible with the vector and not deleterious to the recipients thereof. Typically, the carriers, diluents, and excipients will be saline or infusion media which will be sterile and pyrogen free, however, other acceptable carriers, diluents, and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as (or in addition to) the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The vector, cell, or pharmaceutical composition according to the present invention may be administered in a manner appropriate for treating and/or preventing the diseases described herein. Suitable administration routes will be known to the skilled person. The quantity and frequency of administration may be determined by the skilled person, for example depending by such factors as the condition of the subject, and the type and severity of the subject's disease. The pharmaceutical composition may be formulated accordingly.

The vector, cell or pharmaceutical composition according to the present invention may be administered locally to the eye. Suitably, the vector, cell or pharmaceutical composition according to the present invention is administered by subretinal injection, direct retinal injection, subconjunctivital injection, sub-Tenon's injection, periocular injection, suprachoroidal injection, or intravitreal injection. The pharmaceutical composition may be formulated accordingly.

In some embodiments, the vector, cell, or pharmaceutical composition of the present invention is administered intraocularly. The term “intraocular” may refer to the interior of the eye, thus intraocular administration may relate to the administration to the interior of the eye of a subject. In some embodiments, the vector, cell, or pharmaceutical composition is administered to the eye of a subject by subretinal, direct retinal, suprachoroidal, or intravitreal injection. The skilled person will be familiar with and well able to carry out individual subretinal, direct retinal, suprachoroidal, or intravitreal injections (see e.g. Hartman, R. R. and Kompella, U. B., 2018. Journal of Ocular Pharmacology and Therapeutics, 34 (1-2), pp. 141-153).

In some embodiments, the vector, cell, or pharmaceutical composition is administered to the eye of a subject by subretinal, suprachoroidal, or intravitreal injection. In preferred embodiments, the vector, cell, or pharmaceutical composition of the present invention is administered by intravitreal injection.

The pharmaceutical compositions may comprise vectors or cells of the invention in infusion media, for example sterile isotonic solution. The pharmaceutical composition may be enclosed in ampoules, disposable syringes or multiple dose vials made of glass or plastic.

The vector, cell or pharmaceutical composition may be administered in a single or in multiple doses. Suitably, the vector, cell or pharmaceutical composition may be administered in a single, one off dose. The pharmaceutical composition may be formulated accordingly.

The vector, cell or pharmaceutical composition may be administered at varying doses (e.g. measured in viral genomes (vg) per mL). The physician in any event may determine the actual dosage which will be most suitable for any individual subject and the dosage may, for example, vary with the age, weight and response of the particular subject.

Suitably, the vector of the present invention is administered at a dose of at least about 10¹⁰ vg/mL, at least about 10¹¹ vg/mL, at least about 10¹² vg/mL, or at least about 5×10¹² vg/mL. Suitably, the vector of the present invention is administered at a dose of about 10¹³ vg/mL or less, about 10¹² vg/mL or less, or about 10¹¹ vg/mL or less. Suitably, the vector of the present invention is administered in a dose of from about 10¹⁰ to about 10¹³ vg/mL, or from about 10¹¹ to about 10¹³ vg/mL. Suitably, the vector of the present invention is administered in a dose of from about 10¹⁰ to about 10¹² vg/mL. Suitably, the vector of the present invention is administered in a dose of from about 10¹¹ to about 10¹³ vg/mL. Suitably, the vector of the present invention is administered in a dose of from about 10¹² to about 10¹³ vg/mL. Suitably, the vector of the present invention is administered in a dose of from about 10¹² to about 5×10¹² vg/mL. The pharmaceutical composition may be formulated accordingly.

Suitably, the vector of the present invention is administered at a dose of at least about 109 vg/eye, at least about 2×109 vg/eye, at least about 5×109 vg/eye, at least about 10¹⁰ vg/eye, at least about 2×10¹⁰ vg/eye, at least about 5×10¹⁰ vg/eye, or at least about 10¹¹ vg/eye. Suitably, the vector of the present invention is administered at a dose of about 10¹³ vg/eye or less or about 5×10¹² vg/eye or less. Suitably, the vector of the present invention is administered at a dose of from about 10° vg/eye to about 5×10¹² vg/eye, from about 10¹⁰ vg/eye to about 5×10¹² vg/eye, from about 10¹⁰ vg/eye to about 10¹² vg/eye, or from about 10¹⁰ vg/eye to about 5×10¹¹ vg/eye.

Suitably, the vector of the present invention may be administered in combination with one or more other therapeutic agents. The one or more other therapeutic agent may be administered separately, simultaneously or sequentially. The pharmaceutical composition may further comprise one or more other therapeutic agents. For example, the vector of the present invention may be administered in combination with one or more immunosuppressant (e.g. antimetabolites, calcineurinic inhibitors, and alkylating agents).

The vector of the present invention may reduce the requirement for immunosuppressant therapy. In some embodiments, the vector of the present invention is administered in the absence of immunosuppressant therapy (i.e. the subject does not undergo an immunosuppressant therapy).

The invention further includes kits comprising the vector, cell and/or pharmaceutical composition of the present invention. Preferably said kits are for use in the methods and used as described herein, e.g., the therapeutic methods as described herein. Preferably said kits comprise instructions for use of the kit components.

Methods for Treating and/or Preventing Disease

In one aspect, the present invention provides the vector, cell and/or pharmaceutical composition according to the present invention for use as a medicament.

In one aspect, the present invention provides use of the vector, cell or pharmaceutical composition according to the present invention in the manufacture of a medicament.

In one aspect, the present invention provides a method of administering a therapeutically effective amount of the vector, cell or pharmaceutical composition according to the present invention to a subject in need thereof.

The vector, cell or pharmaceutical composition may be administered to any subject in need thereof. The subject may be a mammal (e.g. a human). The vector, cell or pharmaceutical composition according to the present invention may be administered to a subject with or at risk of an inflammatory eye disease.

### Inflammatory Eye Diseases

The vector, cell or pharmaceutical composition according to the present invention may be used to prevent and/or treat inflammatory eye diseases.

In one aspect, the present invention provides the vector, cell or pharmaceutical composition according to the present invention for use in preventing and/or treating an inflammatory eye disease.

In one aspect, the present invention provides use of the vector, cell or pharmaceutical composition according to the present invention in the manufacture of a medicament for preventing or treating an inflammatory eye disease.

In one aspect, the present invention provides a method of preventing or treating an inflammatory eye disease, the method comprising administering a therapeutically effective amount of the vector, cell or pharmaceutical composition according to the present invention to a subject in need thereof.

As used herein, an “inflammatory eye disease” may refer to any disorder associated with eye inflammation including uveitis, scleritis, keratitis, conjunctivitis, iritis, chorioretinitis, choroiditis, retinitis, and retinochoroiditis.

Following administration of the vector, cell and/or pharmaceutical composition according to the present invention, one or more symptoms of inflammatory eye disease may be prevented and/or treated in the subject. Any suitable method for determining the severity of inflammatory eye disease may be used (see e.g. McNeil, R., 2016. Eye news, 22 (5), pp. 1-4).

The vector, cell and/or pharmaceutical composition according to the present invention may prevent and/or reduce intraocular inflammation. Any suitable method may be used to determine intraocular inflammation. Suitable methods to quantify intraocular inflammation include laser flare photometry (see e.g. Tugal-Tutkun, I. and Herbort, C. P., 2010. International ophthalmology, 30 (5), pp. 453-464).

The vector, cell and/or pharmaceutical composition according to the present invention may reduce the inflammatory eye disease relapse rate. Any suitable method may be used to determine the relapse rate. For example, a recurrence (or flare) of uveitis is typically defined as an anterior chamber cells and/or vitreous haze grading of ≥2+ using the SUN grading system (see e.g. McNeil, R., 2016. Eye news, 22 (5), pp. 1-4).

The vector, cell and/or pharmaceutical composition according to the present invention may prevent and/or reduce vision loss. Inflammatory eye diseases such as uveitis is a major cause of visual morbidity (see e.g. Durrani, O. M., et al., 2004. British Journal of Ophthalmology, 88 (9), pp. 1159-1162). The vector, cell and/or pharmaceutical composition according to the present invention may maintain or improve visual acuity.

### Uveitis

In preferred embodiments, the inflammatory eye disease is uveitis.

In one aspect, the present invention provides the vector, cell or pharmaceutical composition according to the present invention for use in preventing and/or treating uveitis.

In one aspect, the present invention provides use of the vector, cell or pharmaceutical composition according to the present invention in the manufacture of a medicament for preventing or treating uveitis.

In one aspect, the present invention provides a method of preventing or treating uveitis, the method comprising administering a therapeutically effective amount of the vector, cell or pharmaceutical composition according to the present invention to a subject in need thereof.

Uveitis refers to a group of intraocular inflammatory diseases of the uvea (i.e., the iris, ciliary body, and choroid) and adjacent structures, including the cornea, vitreous humor, retina, and optic nerve. Uveitis can be classified based on the primary anatomical site of inflammation (i.e., anterior, intermediate, and posterior) and/or etiologic origin, including infectious, non-infectious, or masquerade (see e.g. Rosenbaum, J. T., et al., 2019. Seminars in Arthritis and Rheumatism, 49 (3), pp. 438-445; and Standardization of Uveitis Nomenclature (SUN) Working Group, 2005. American journal of ophthalmology, 140 (3), pp. 509-516).

The uveitis may be anterior uveitis, intermediate uveitis, posterior uveitis, or panuveitis. The uveitis may be infectious, non-infectious, or masquerade. In preferred embodiments, the uveitis is non-infectious uveitis.

In some embodiments, the uveitis is selected from one or more of: sympathetic ophthalmia. Birdshot Chorioretinopathy, Sarcoid uveitis, Intermediate uveitis, Vogt Koyanaga Harada syndrome, JIA-associated uveitis, idiopathic retinal vasculitis, HLA-B27 associated non-anterior uveitis.

In some embodiments, the uveitis is sympathetic ophthalmia. Sympathetic ophthalmia is a rare, bilateral granulomatous uveitis that occurs after either surgical or accidental trauma to one eye (see e.g. Damico, F. M., et al., 2005. Seminars in ophthalmology, 20 (3), pp. 191-197).

In some embodiments, the uveitis is Birdshot Chorioretinopathy. Birdshot retinochoroidopathy is a rare, chronic, bilateral, posterior uveitis (see e.g. Levinson, R. D., et al., 2006. American journal of ophthalmology, 141 (1), pp. 185-187).

In some embodiments, the uveitis is sarcoid uveitis. Sarcoid uveitis may also be known as sarcoid-related uveitis. Sarcoidosis is a disease that causes noncaseating granulomatous inflammation in one or more organs. The most common ocular manifestations are uveitis, dry eye and conjunctival nodules (see e.g. Jamilloux, Y., et al., 2014. Autoimmunity reviews, 13 (8), pp. 840-849).

In some embodiments, the uveitis is intermediate uveitis. Intermediate uveitis may refer to inflammation in the anterior vitreous, ciliary body and the peripheral retina (see e.g. Babu, B. M. and Rathinam, S. R., 2010. Indian journal of ophthalmology, 58 (1), p. 21).

In some embodiments, the uveitis is Vogt Koyanaga Harada syndrome. Vogt-Koyanagi-Harada syndrome is a bilateral, chronic, diffuse granulomatous panuveitis frequently associated with neurological, auditory, and integumentary manifestations (see e.g. Fang, W. and Yang, P., 2008. Current eye research, 33 (7), pp. 517-523).

In some embodiments, the uveitis is JIA-associated uveitis. JIA is the most common rheumatic disease of childhood, with JIA-associated uveitis its most common extra-articular manifestation (see e.g. Clarke, S. L., et al., 2016. Pediatric Rheumatology, 14 (1), pp. 1-11).

In some embodiments, the uveitis is idiopathic retinal vasculitis. Retinal vasculitis is a sight-threatening inflammatory eye condition that involves the retinal vessels. Based on the etiology, retinal vasculitis may be classified as either idiopathic or secondary to infection, neoplasia, or a systemic inflammatory disease (see e.g. Talat, L., et al., 2014. Journal of ophthalmology, 197675).

In some embodiments, the uveitis is HLA-B27 associated non-anterior uveitis. Human Leukocyte Antigen (HLA)-B27-associated uveitis is the most commonly diagnosed cause of acute anterior uveitis (see e.g. Loh, A. R. and Acharya, N. R., 2010. American journal of ophthalmology, 150 (4), pp. 534-542).

### EXAMPLES

The invention will now be further described by way of examples, which are meant to serve to assist one of ordinary skill in the art in carrying out the invention and are not intended in any way to limit the scope of the invention.

### Example 1—Local Anti-TNF Antibody Fragment Administration Suppresses Experimental Autoimmune Uveoretinitis (EAU) In Vivo

B10.RIII mice were immunized for Experimental Autoimmune Uveoretinitis (EAU) and eyes monitored using Topical Endoscopic Fundal Imaging (TEFI) from day 10 onward to select experimental mice displaying clinically evident disease. Groups of mice were injected via intravitreal route with 15 μg infliximab or vehicle control (EAU) on day 10. Eyes were enucleated (day 14), and retinal infiltrate characterized. Representative fundus images (FIG. 1A), clinical disease scores (FIG. 1B) and flow cytometric analysis of total CD45+ cell numbers from single eyes at day 14 (FIG. 1C), demonstrate efficacy of intravitreal Infliximab derived Fab molecules to suppress inflammation and retinal infiltrate in the B10.RIII EAU model. **P<0.005; Data presented as means+/−SEM, representative of two independent experiments.

### Example 2—Therapeutic Vector Design

FIG. 2 is a schematic showing the vector organization of CMV. Infliximab Fab (constitutive CMV promoter) and AP1-NFkB. Infliximab Fab (inflammation-inducible promoter comprised of 5 repeated AP1 and NFkB binding sites). The heavy and light chains of Infliximab Fab are separated by a self-cleaving 2A peptide to generate the two separate peptides, which then form the Fab in situ. Following transduction of cells, AAV persists as episomal DNA in the nucleus.

### Example 3—Evaluation of Constitutive Transgene Expression In Vitro and In Vivo

To evaluate constitutive Infliximab Fab transgene expression, HEK-293T (standard cell line for AAV development) or ARPE-19 (ocular cell line) cells were transduced with AAV2.CMV. Infliximab or AAV.CMV.NULL vectors [MOI 1E5 vg/cell], and culture supernatants assayed using a clinical IFX ELISA kit (FIG. 3A). Detectable expression of Infliximab Fab from both cell types was evident by 72 hrs (˜30 ng/ml). ****P<0.0001. Data presented as means+/−SEM.

To evaluate in vivo transgene expression, AAV2.CMV. Infliximab or AAV.CMV.NULL was administered by intravitreal (IVT) injection at 5E12 vg/ml into eyes of healthy B10.RIII mice. At 4 wks post-AAV, mice were killed, eyes dissected and supernatants (retina and vitreous) assayed using the clinical IFX ELISA kit (FIG. 3B). Detectable expression of in vivo Infliximab Fab (˜1.5 ng/ml) was observed in eyes receiving the 5E12 vg/ml dose. *P<0.05. Data presented as means+/−SEM, with each data point representing a single eye.

These data demonstrate that AAV can mediate constitutive expression of an anti-TNF Fab.

### Example 4—Evaluation of Inducible Transgene Expression In Vitro and In Vivo Clinical Model

To demonstrate inducibility of the inflammation responsive promotor design, HEK-293T cells transduced with AAV2.AP1-NFkB.EGFP (reporter vector) or AAV2.AP1-NFkB. Infliximab (therapeutic vector) were stimulated with recIL-1b (2 ng/ml). Activation leads to visible GFP expression at 24 hs, increasing in intensity by 72 hrs, with no GFP signal observed with AAV2.AP1-NFkB.NULL (control vector) (FIG. 4A). A higher GFP signal is observed with the constitutive AAV.CMV.EGFP vector. Stimulation results in a rapid induction of Infliximab Fab expression (8 hrs), accumulation reaching ˜20 ng/ml at 72 hrs (FIG. 4B). Images captured on EVOS FL, 10× magnification. ****P<0.0001; Data presented as means+/−SEM.

To demonstrate in vivo inducibility of the inflammation responsive promotor design, AAV2.AP1-NFkB.EGFP (reporter) or AAV.AP1-NFkB.NULL (control) at a 5E12 vg/ml dose was administered by intravitreal (IVT) injection C57BL/6J mice. At 4 wks post-AAV injection, mice were immunized to induce experimental autoimmune uveoretinitis (EAU), and imaged to monitor onset of ocular inflammation. At day 14 EAU, representative fundus and OCT images demonstrate clear clinical signs of disease (perivascular sheathing and vitreous infiltrate), and induction of GFP expression (FIG. 5A). In mice that only received AAV (no EAU), no clinical signs of disease or expression of the GFP transgene are observed.

To demonstrate in vivo inducibility of the therapeutic transgene, groups of mice were injected with AAV2.AP1-NFkB. Infliximab or AAV2.AP1-NFkB.NULL in the contralateral eye, and EAU induced at 4 wks post-AAV. At 3 wks post-EAU, mice were killed, eyes dissected and supernatants (retina and vitreous) assayed using the clinical IFX ELISA kit (FIG. 5B). Detectable expression of the Infliximab Fab is only observed in EAU eyes receiving the therapeutic vector and not the control. *P<0.05. Data presented as means+/−SEM, with each data point representing a single eye.

These data show inducibility of the inflammation responsive promotor design to express either a reporter (eGFP) or therapeutic (anti-TNF Fab) transgene in vitro and in vivo.

### Example 5—Evaluation of Constitutive Therapeutic Transgene Efficacy In Vivo

To demonstrate efficacy of the constitutive therapeutic transgene, groups of mice were injected with AAV7m8.CMV. Infliximab or AAV7m8.CMV.NULL in the contralateral eye, followed by intravitreal administration of recombinant human TNF (rec_hTNF) at 4 wks post-AAV. At 18 hrs (peak of inflammatory response to rec_hTNF), representative fundus and OCT infliximab eyes (FIG. 6A). In mice that only received AAV, no clinical signs of disease are observed. At 18 hrs mice were killed, eyes dissected prepared for flow cytometric analysis to determine absolute numbers of Ly6C+ monocytes (predominant infiltrate in this model) from single eyes. A significant reduction in number of monocytes was observed in eyes receiving the therapeutic vector and not the control (FIG. 6B), this effect is further highlighted further with paired (contralateral eye) analysis (FIG. 6C). **Wilcoxon signed rank test; *P<0.05 Wilcoxon matched pairs analysis.

These data show efficacy of the constitutive promotor design encoding a therapeutic (Infliximab Fab) transgene, to suppress human TNF mediated inflammation in vivo.

### Example 6—Constitutive Expression of Other Anti-TNF Biologics In Vitro and In Vivo

To evaluate constitutive expression of additional anti-TNF biologics, HEK-293T cells were transfected with the huTNFRI-huIgG plasmid, and culture supernatants assayed using an anti-human TNF antibody ELISA kit (FIG. 7A). Detectable expression of huTNFRI-huIgG was evident by 48 hrs. To evaluate AAV-mediated transgene expression, HEK-293T cells were transduced with AAV7m8.CMV.huTNFRI-huIgG or AAV7m8.CMV.NULL vectors [MOI 1E5 vg/cell], and culture supernatants assayed using an anti-human TNF antibody ELISA kit (FIG. 7B). Detectable expression of huTNFRI-IgG was evident by 72 hrs (˜30 ng/ml).

To evaluate in vivo constitutive transgene expression of the huTNFRI-huIgG, AAV7m8.CMV.huTNFRI-huIgG or AAV7m8.CMV.NULL was administered by intravitreal (IVT) injection at range of doses [2E8 or 2E9 vg/eye] into the eyes of healthy C57BL/6J mice. At 4 wks post-AAV, mice were killed, eyes dissected and supernatants (retina and vitreous) assayed using the anti-human TNF antibody ELISA kit (FIG. 7C). Detectable expression in vivo of huTNFRI-huIgG at ˜4 ng/ml and 9 ng/ml was observed in eyes receiving 2E8 and 2E9 vg/eye doses respectively.

These data provide additional evidence that gene therapy vectors can mediate constitutive expression of multiple clinically relevant anti-TNF biologics.

### Example 7—Bioactivity of Constitutively Expressed Anti-TNF Transgenes In Vitro

To determine whether the transgene products are bioactive and functional, we evaluated AAV plasmids encoding Adalimumab Fab, huTNFRI-huIgG, and two modified constructs engineered to express a huTNFRI ectodomain fused to mouse Fc (huTNFRI-msIgG) and a murine version (msTNFRI-msIgG). We also evaluated bioactivity of Infliximab Fab using the AAV2.CMV. Infliximab viral vector.

HEK-BLUE TNF reporter cells were first transfected with AAV.CMV.huTNFRI-huIgG, AAV.CMV.huTNFRI-msIgG, AAV.CMV.msTNFRI-msIgG, AAV.CMV.ADALIMUMAB plasmids or AAV2.CMV. Infliximab for 48 hrs. Cells were stimulated with recombinant human TNF or mouse TNF (0.5 ng/ml) for a further 24 hours and NFkB activation assessed. All the TNFRI antibody-like plasmid constructs were bioactive, inhibiting huTNF-and msTNF-mediated activation in the reporter cell line compared to NULL or recTNF alone (FIG. 8A). For the monoclonal Fab based anti-TNF biologics (both human specific), plasmid expression of Adalimumab Fab (FIG. 8B) or AAV-mediated Infliximab Fab expression (FIG. 8C) both inhibit activation with huTNF.

These data demonstrate vectorized anti-TNF biologics, including huTNFRI-huIgG and Adalimumab are bioactive, and neutralize TNF mediated signalling and NFkB activation in vitro.

### Example 8—Evaluation of Expression and Bioactivity of an Inducible Anti-TNF Transgene In Vitro and In Vivo Clinical Disease Model

To further demonstrate inducibility of anti-TNF biologics via an inflammation responsive promotor, HEK-293T cells were transduced with AAV7m8.AP1-NFkB.huTNFRI-huIgG or AAV7m8.AP1-NFkB.NULL vectors [MOI 1E5 vg/cell] and stimulated with IL-1b (2 ng/ml). We selected this construct for further characterization as huTNFRI-huIgG can potently neutralize both human and mouse TNF, facilitating efficacy testing in mice in vivo. Stimulation results in robust induction of huTNFRI-huIgG expression by 24 hrs, accumulation reaching ˜25 ng/ml at 48 hrs (FIG. 9A).

To determine bioactivity of induced huTNFRI-huIgG protein, conditioned media (cell supernatant collected from the expression assay at 48 hrs), was “spiked” with recombinant human or mouse TNF (final concentration 10 ng/ml), and incubated with HEK-BLUE reporter cells for 24 hrs. In response to both huTNF or mTNF stimulation, or with conditioned media from AAV7m8.AP1-NFkB.NULL, NFkB activation is robustly induced in the reporter cells. When supplemented with conditioned media from cells transduced with AAV7m8.AP1-NFkB.huTNFRI-huIgG and stimulated with IL-1b, NFkB activation in the reporter cells is completely suppressed, indicating potent bioactivity of the induced transgene (FIG. 9B).

To demonstrate in vivo inducibility of the therapeutic transgene, mice were injected with AAV7m8.AP1-NFkB.huTNFRI-huIgG, and EAU induced at 4 wks post-AAV. At day 19 post-EAU, when mild to moderate clinical signs of inflammation (not yet peak disease) were observed, mice were killed, eyes dissected and supernatants (retina and vitreous) assayed using the anti-human TNF antibody ELISA. We found that clinical disease drove detectable expression of the huTNFRI-huIgG in EAU eyes receiving the therapeutic vector and not the control (AAV only) (FIG. 10A).

Using the recombinant human TNF model, we also evaluated inducibility of the huTNFRI-huIgG transgene in response to an acute inflammatory stimulus. Mice were injected with AAV7m8.AP1-NFkB.huTNFRI-huIgG and AAV7m8.AP1-NFkB.NULL (contralateral eye control) at 2E9 vg/eye. At 4 wks post-AAV, mice received bilateral administration of recombinant human TNF (rec_hTNF), 18 hrs later were killed, eyes dissected and ocular supernatants (retina and vitreous) assayed for huTNFRI-huIgG expression. Acute activation elicits a significant increase in expression of the huTNFRI-huIgG compared to the control (FIG. 10B).

These data show inducibility and sensitivity of the inflammation responsive promotor design to express a bioactive therapeutic (anti-TNF biologic) transgene in vitro and in vivo, using two different disease models, one with chronic low-grade inflammation and other with acute ocular inflammation.

### Example 9—Evaluation of Inducible Therapeutic Transgene Efficacy In Vivo

To demonstrate efficacy of the inducible therapeutic transgene, B10.RIII mice were injected with AAV7m8.AP1-NFkB.huTNFRI-huIgG or AAV7m8.CMV.NULL [2E9 vg/eye] in contralateral eyes, and then immunized for EAU at 4 wks post-AAV. At day 11, representative fundus and OCT images demonstrate increased inflammation (peri-vascular sheathing and vitreous infiltrate) in the eyes receiving the control NULL vector (FIG. 11A). The contralateral eyes of the same four animals, which received the inducible therapeutic vector, showed substantially reduced clinical inflammation, both peri-vascular sheathing and vitreous infiltrate. At this time-point mice were killed, eyes dissected prepared for flow cytometric analysis to determine absolute numbers of CD45+ (all leukocytes), CD3+ (lymphocytes), CD4+ (Th T cells) and CD11b+ (macrophages and monocytes) populations from single eyes (FIG. 11B). We observed a trend of reduction in immune cell infiltrates with AAV7m8.AP1-NFkB.huTNFRI-huIgG compared to AAV7m8.CMV.NULL.

These data show efficacy of inducible anti-TNF to locally suppress ocular inflammation in a mouse model of human uveitis in vivo.

### Example 10—Methods

Viral vectors: Design, cloning and sequencing of plasmids performed in-house. Production, QC and quantification of ultra-pure AAV2 preparations performed by Vector Builder. Production, QC and quantification of ultra-pure AAV7m8 preparations performed by Vector BioLabs.

| Name | Promotor | Transgene | Vector Type |
|---|---|---|---|
| AAV2.CMV.Infliximab | Constitutive | Infliximab Fab | Therapeutic |
| AAV2.CMV.NULL | Constitutive | Non-coding sequence | Control |
| AAV2.AP1-NFkB.Infliximab | Inducible | Infliximab Fab | Therapeutic |
| AAV2.AP1-NFkB.EGFP | Inducible | Enhanced GFP | Reporter |
| AAV2.AP1-NFkB.NULL | Inducible | Non-coding sequence | Control |
| AAV7m8.CMV.huTNFRI-huIgG | Constitutive | huTNFRI-huIgG | Therapeutic |
| AAV7m8.CMV.huTNFRI-msIgG | Constitutive | huTNFRI-msIgG | Therapeutic |
| AAV7m8.CMV.msTNFRI-msIgG | Constitutive | msTNFRI-msIgG | Therapeutic |
| AAV7m8.CMV.Adalimumab | Constitutive | Adalimumab Fab | Therapeutic |
| AAV7m8.CMV.NULL | Constitutive | Non-coding sequence | Control |
| AAV7m8.AP1-NFkB.huTNFRI-huIgG | Inducible | huTNFRI-huIgG | Therapeutic |
| AAV7m8.AP1-NFkB.huTNFRI-msIgG | Inducible | huTNFRI-msIgG | Therapeutic |
| AAV7m8.AP1-NFkB.msTNFRI-msIgG | Inducible | msTNFRI-msIgG | Therapeutic |
| AAV7m8.AP1-NFkB.Adalimumab | Inducible | Adalimumab Fab | Therapeutic |
| AAV7m8.AP1-NFkB.NULL | Inducible | Non-coding sequence | Control |

Cell lines: HEK 293T or ARPE-19 cells cultured using standard complete medium. Plasmid transduction of cells performed using lipofectamine at concentration of 0.25 ug/ml. AAV transduction of cells performed at range of Multiplicity of Infection (MOI; 1E4-1E5 vg/cell).

Anti-TNF constructs: Infliximab Fab was expressed via encoding the heavy and light chains of Infliximab Fab separated by a self-sleaving 2A peptide to generate the two separate peptides, which then form the Fab in situ. Adalimumab Fab was expressed similarly, but encoding the heavy and light chains of adalimumab Fab. For both Fabs, a Human Growth Hormone (HGH) signal peptide was added to the N-terminus of both heavy chain and light chain to allow secretion.

For antibody-like molecules, huTNFRI-huIgG was expressed by encoding the extracellular domain (N-terminus 211 amino acid residues, which include a native signal peptide) of human tumour necrosis factor receptor type 1 (TNFRI, also known as p55 receptor), followed by the Fc region of human IgG1. Similarly, huTNFRI-msIgG was expressed, but switching the Fc region of human IgG1 for that of mouse IgG1. Similarly, msTNFRI-msIgG was expressed, but additionally switching the extracellular domain (N-terminus 211 amino acid residues, which include a native signal peptide) of huTNFRI for the extracellular domain (N-terminus 212 amino acid residues, which include a native signal peptide) of msTNFRI. These soluble fusion proteins bind to and neutralize TNF.

Infliximab Fab and huTNFRI-huIgG Characterization: Levels of secreted Fab or IgG (cell culture or ex vivo retinal supernatants) assayed using a clinical IFX ELISA kit (R-Biopharm) or Anti-human TNF antibody ELISA kit (LS Bio; LS-F55832). Bioactivity of Infliximab Fab or huTNFRI-huIgG (i.e. the ability to neutralize human/mouse TNF) evaluated using TNF-α Reporter HEK 293 cells (Invivogen), which permits monitoring of NF-κB pathway activation.

Therapeutic intervention (AAV or biologics): Intravitreal injections performed under ketamine-based recovery anaesthesia, with an operating microscope using a 33G Hamilton syringe to inject up to 2 μl volumes.

Uveitis model: Experimental Autoimmune Uveoretinitis (EAU) is an established preclinical model of human non-infectious Uveitis (Khalili, H., et al., Sci Rep, 2016. 6: p. 36905). Induced using standard immunizing protocol, with retinal peptides (RBP-3), with additional adjuvant (CFA and Pertussis Toxin) by appropriate systemic route (e.g. subcutaneous or intraperitoneal injection). EAU susceptibility is mouse strain dependent, with different RBP-3 epitopes eliciting acute, severe inflammation (B10.RIII) or persistent disease with reduced disease severity (C57BL/6J). Initial therapeutic AAV2. Infliximab vector testing uses the B10.RIII strain, and subsequent therapeutic AAV7m8.huTNFRI-huIgG vector used both strains.

Recombinant human TNF model: To evaluate efficacy of therapeutic anti-TNF vectors to human protein, acute inflammation can be induced by intravitreal injection of recombinant human TNF (20 ng/eye). Susceptibility is not mouse strain dependent, with similar inflammatory kinetics in B10.RIII or C57BL/6J.

Clinical assessment: The Micron IV system permits repeated in vivo ocular assessment (Fundus, Fluorescence, OCT & ERG) of disease severity. Ex vivo, single retinas are processed for routine 15-colour flow cytometry (FACS) to immuno-phenotype and quantify CD45+ infiltrate cell populations.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example: Skoog, D. A., et al. (2013) Fundamentals of Analytical Chemistry, 9th edition, Cengage learning; Walker J. M. (2009) The Protein Protocols Handbook, 3rd edition, Springer Nature; Green, M. R. and Sambrook, J. (2012) Molecular Cloning: A Laboratory Manual, 4th Edition, Cold Spring Harbor Laboratory Press; Ausubel, F. M., et al. (2003) Current Protocols in Molecular Biology, John Wiley & Sons; Hill, A. J. (2013) DNA Sequencing Protocols, Humana Press; Nielsen, B. S. and Jones, J. (2021) In Situ Hybridization Protocols, Springer US; Herdewijn, P. (2010) Oligonucleotide Synthesis: Methods and Applications, Humana Press; and Luo, Y. (2019) CRISPR Gene Editing: Methods and Protocols, Springer New York. Each of these general texts is herein incorporated by reference.

1. A vector comprising a nucleotide sequence encoding an anti-TNF antibody or a fragment thereof, wherein the nucleotide sequence encoding the anti-TNF antibody or a fragment thereof is operably linked to an inflammation-inducible promoter.

2. The vector according to claim 1, wherein the anti-TNF antibody or fragment thereof is an antibody fragment, preferably wherein the antibody fragment is an antigen-binding fragment (Fab), a fragment antibody (F(ab′)₂), a single chain antibody (scFv), or a single-domain antibody (sdAb).

3. A vector comprising a nucleotide sequence encoding an anti-TNF antibody fragment selected from an antigen-binding fragment (Fab), a fragment antibody (F(ab′)₂), a single chain antibody (scFv), and a single-domain antibody (sdAb).

4. The vector according to claim 3, wherein the nucleotide sequence encoding an anti-TNF antibody fragment is operably linked to an inflammation-inducible promoter.

5. The vector according to any preceding claim, wherein the anti-TNF antibody or fragment thereof is any of adalimumab or a fragment thereof, infliximab or a fragment thereof, golimumab or a fragment thereof, or certolizumab or a fragment thereof.

6. The vector according to any preceding claim, wherein the anti-TNF antibody or fragment thereof is adalimumab or a fragment thereof, or infliximab or a fragment thereof.

7. The vector according to any preceding claim, wherein the anti-TNF antibody or fragment thereof is adalimumab or a fragment thereof.

8. The vector according to any preceding claim, wherein the anti-TNF antibody or fragment thereof is an antigen-binding fragment (Fab).

9. The vector according to any preceding claim, wherein the anti-TNF antibody or fragment thereof is an antigen binding fragment (Fab) of adalimumab.

10. The vector according to any preceding claim, wherein the anti-TNF antibody or fragment thereof comprises one or more CDR regions selected from SEQ ID NOs: 1 to 6 or derivatives thereof comprising one amino acid substitution.

11. The vector according to any preceding claim, wherein the anti-TNF antibody or fragment thereof comprises CDR regions HCDR1, HCDR2, HCDR3, LCDR1, LCDR2, LCDR3 comprising or consisting of SEQ ID NOs: 1, 2, 3, 4, 5 and 6 respectively or derivatives thereof comprising one amino acid substitution.

12. The vector according to any preceding claim, wherein the anti-TNF antibody or fragment thereof comprises a heavy chain comprising or consisting of a sequence with at least 70% identity to SEQ ID NO: 7 and/or a light chain comprising or consisting of a sequence with at least 70% identity to SEQ ID NO: 8.

13. The vector according to claim 12, wherein the heavy chain is encoded by a nucleotide sequence having at least 70% identity to SEQ ID NO: 47 and/or the light chain is encoded by a nucleotide sequence having at least 70% identity to SEQ ID NO: 48.

14. The vector according to claim 13, wherein the nucleotide sequence encoding the heavy chain and the nucleotide sequence encoding the light chain are connected via a linker sequence.

15. The vector according to claim 14, wherein the linker sequence encodes a 2A self-cleaving peptide, and/or an enzymatically cleavable peptide motif, preferably wherein the linker sequence encodes a 2A self-cleaving peptide having at least 70% sequence identity to any of SEQ ID NOs: 55-58.

16. The vector according to any of claims 13 to 15, wherein the nucleotide sequence encoding the heavy chain and/or the nucleotide sequence encoding the light chain is operably linked to a signal sequence, optionally wherein the signal sequence encodes a signal peptide selected from any of: a Human Growth Hormone (HGH) signal peptide, an interleukin-2 (IL-2) signal peptide, a CD5 signal peptide, an immunoglobulin Kappa light chain signal peptide, a trypsinogen signal peptide, a serum albumin signal peptide, and a prolactin signal peptide.

17. The vector according to any preceding claim, wherein the nucleotide sequence encoding an anti-TNF antibody or a fragment encodes a heavy chain comprising or consisting of a sequence with at least 70% identity to SEQ ID NO: 7, a 2A self-cleaving peptide having at least 70% sequence identity to any of SEQ ID NOs: 55-58, and a light chain comprising or consisting of a sequence with at least 70% identity to SEQ ID NO: 8.

18. The vector according to any preceding claim, wherein the nucleotide sequence encoding an anti-TNF antibody or a fragment thereof comprises or consists of: a nucleotide sequence having at least 70% identity to SEQ ID NO: 47, a nucleotide sequence having at least 70% sequence identity to SEQ ID NO: 59 or 60, and a nucleotide sequence having at least 70% identity to SEQ ID NO: 48.

19. The vector according to any preceding claim, wherein the nucleotide sequence encoding an anti-TNF antibody or a fragment thereof comprises or consists of a nucleotide sequence having at least 70% identity to SEQ ID NO: 66.

20. The vector according to any of claim 1, 2 or 4-19, wherein the inflammation-inducible promoter comprises one or more inflammation-inducible transcription factor binding motif selected from: an AP-1 transcription factor binding motif; a NF-κB transcription factor binding motif; an IRF transcription factor binding motif; a STAT transcription factor binding motif; and a NFAT transcription factor binding motif or any combination thereof.

21. The vector according to any of claim 1, 2 or 4-20, wherein the inflammation-inducible promoter comprises one or more AP-1 binding motif and/or one or more NF-κB binding motif.

22. The vector according to any of claim 1, 2 or 4-21, wherein the inflammation-inducible promoter comprises two or more AP-1 binding motifs and/or two or more NF-κB binding motifs, three or more AP-1 binding motifs and/or three or more NF-κB binding motifs, four or more AP-1 binding motifs and/or four or more NF-κB binding motifs, or five or more AP-1 binding motifs and/or five or more NF-κB binding motifs

23. The vector according to any of claim 1, 2 or 4-22, wherein the inflammation-inducible promoter comprises at least one AP-1 binding motif coupled to at least one NF-κB binding motif.

24. The vector according to any of claim 1, 2 or 4-23, wherein the inflammation-inducible promoter comprises five AP-1 binding motifs coupled to five NF-κB binding motifs.

25. The vector according to any of claims 20-24, wherein the AP-1 binding motif comprises or consists of SEQ ID NO: 70, or wherein the AP-1 binding motif comprises or consists of any of SEQ ID NOs: 71-73 or derivatives thereof comprising one nucleotide substitution.

26. The vector according to any of claims 20-25, wherein the NF-κB binding motif comprises or consists of SEQ ID NO: 74, or wherein the NF-κB binding motif comprises or consists of SEQ ID NO: 75 or a derivative thereof comprising two or fewer nucleotide substitutions.

27. The vector according to any of claim 1, 2 or 4-26, wherein the inflammation-inducible promoter comprises or consists of a nucleotide sequence having at least 70% identity to SEQ ID NO: 76.

28. The vector according to any preceding claim, wherein the vector comprises a nucleotide sequence having at least 70% identity to SEQ ID NO: 77.

29. The vector according to any preceding claim, wherein the nucleotide sequence encoding the anti-TNF antibody or a fragment thereof is operably linked to a polyadenylation sequence, optionally wherein the polyadenylation sequence is selected from any of: a bovine growth hormone (bGH) polyadenylation sequence, a SV40 polyadenylation sequence, and a rabbit beta-globin polyadenylation sequence.

30. The vector according to claim 29, wherein the polyadenylation sequence comprises or consists of a nucleotide sequence having at least 70% identity to SEQ ID NO: 78.

31. The vector according to any preceding claim, wherein the nucleotide sequence encoding the anti-TNF antibody or a fragment thereof is operably linked to a woodchuck hepatitis post-transcriptional regulatory element (WPRE).

32. The vector according to claim 31, wherein the WPRE comprises or consists of a nucleotide sequence having at least 70% identity to SEQ ID NO: 79.

33. The vector according to any preceding claim, wherein the nucleotide sequence encoding the anti-TNF antibody or a fragment thereof is operably linked to an intron, optionally wherein the intron is selected from a beta-globin intron or a SV40 intron.

34. The vector according to claim 33, wherein the intron comprises or consists of a nucleotide sequence having at least 70% identity to SEQ ID NO: 80.

35. The vector according to any previous claim, wherein the vector is a viral vector.

36. The vector according to any previous claim, wherein the vector is any of a parvoviral vector, an adenoviral vector, a herpes simplex viral vector, an anelloviral vector, a retroviral vector or a lentiviral vector, preferably wherein the vector is an adeno-associated virus (AAV) vector.

37. The vector according to any previous claim, wherein the vector is an AAV vector particle.

38. The vector according to claim 37, wherein the AAV vector particle is pseudotyped to confer ocular tissue tropism.

39. The vector according to claim 37 or 38, wherein the AAV vector particle comprises AAV2 capsid proteins or AAV2 capsid variant proteins, optionally wherein the AAV2 capsid variant is selected from any of: AAV2.tYF, AAV2.7m8, R100, AAV2.GL and AAV2.NN.

40. The vector according to any previous claim, wherein the vector comprises one or more inverted terminal repeats (ITRs).

41. The vector according to any previous claim, wherein the vector comprises a nucleotide sequence having at least 70% identity to SEQ ID NO: 91.

42. A vector comprising a nucleotide sequence having at least 70% identity to SEQ ID NO: 91.

43. An isolated cell comprising the vector according to any of claims 1-42.

44. A kit for the production of the vector of any one of claims 1-42.

45. A pharmaceutical composition comprising the vector according to any one of claims 1 to 42 or the isolated cell according to claim 43, in combination with a pharmaceutically acceptable carrier, diluent or excipient.

46. A vector according to any one of claims 1 to 42, an isolated cell according to claim 43, and/or a pharmaceutical composition according to claim 45, for use as a medicament.

47. Use of a vector according to any one of claims 1 to 42, an isolated cell according to claim 43, or a pharmaceutical composition according to claim 45, for the manufacture of a medicament.

48. A method comprising administering a vector according to any one of claims 1 to 42, an isolated cell according to claim 43, or a pharmaceutical composition according to claim 45, to a subject in need thereof.

49. A vector for use in preventing or treating an inflammatory eye disease, wherein the vector comprises a nucleotide sequence encoding a TNF inhibitor, and wherein the nucleotide sequence encoding the TNF inhibitor is operably linked to an inflammation-inducible promoter.

50. Use of a vector in the manufacture of a medicament for preventing or treating an inflammatory eye disease, wherein the vector comprises a nucleotide sequence encoding a TNF inhibitor, and wherein the nucleotide sequence encoding the TNF inhibitor is operably linked to an inflammation-inducible promoter.

51. A method for preventing or treating an inflammatory eye disease, wherein the method comprises administering a vector to a subject in need thereof, wherein the vector comprises a nucleotide sequence encoding a TNF inhibitor, and wherein the nucleotide sequence encoding the TNF inhibitor is operably linked to an inflammation-inducible promoter.

52. A vector according to any one of claims 1 to 42, or a pharmaceutical composition according to claim 45, for use in preventing or treating an inflammatory eye disease.

53. Use of a vector according to any one of claims 1 to 42, or a pharmaceutical composition according to claim 45, for the manufacture of a medicament for preventing or treating an inflammatory eye disease.

54. A method of preventing or treating an inflammatory eye disease comprising administering a vector according to any one of claims 1 to 42, or a pharmaceutical composition according to claim 45, to a subject in need thereof.

55. The vector or pharmaceutical composition for use according to claim 49 or 52, the use according to claim 50 or 53, or the method according to claim 51 or 54, wherein the inflammatory eye disease is uveitis.

56. The vector or pharmaceutical composition for use according to any of claim 49, 52 or 55, the use according to any of claim 50, 53 or 55, or the method according to any of claim 51 or 54-55, wherein the vector or pharmaceutical composition is administered intraocularly.

57. The vector or pharmaceutical composition for use according to any of claim 49, 52, or 55-56, the use according to any of claim 50, 53 or 55-56, or the method according to any of claim 51 or 54-56, wherein the vector or pharmaceutical composition is administered via intravitreal, subretinal, direct retinal, subconjunctivital, sub-Tenon's or suprachoroidal injection.

58. The vector or pharmaceutical composition for use according to any of claim 49, 52, or 55-57, the use according to any of claim 50, 53 or 55-57, or the method according to any of claim 51 or 54-57, wherein the vector or pharmaceutical composition is administered via intravitreal injection.

59. The vector or pharmaceutical composition for use according to any of claim 49, 52, or 55-58, the use according to any of claim 50, 53 or 55-58, or the method according to any of claim 51 or 54-58, wherein the vector or pharmaceutical composition is administered as a single dose.

60. The vector or pharmaceutical composition for use according to any of claim 49, 52, or 55-59, the use according to any of claim 50, 53 or 55-59, or the method according to any of claim 51 or 54-59, wherein the vector is administered at a dose of at least about 1E10 vg/mL, at least about 1E11 vg/mL, at least about 1E12 vg/mL, or at least about 5E12 vg/mL.

61. The vector or pharmaceutical composition for use according to any of claim 49, 52, or 55-60, the use according to any of claim 50, 53 or 55-60, or the method according to any of claim 51 or 54-60, wherein the vector is administered at a dose of at least about 1E9 vg/eye, at least about 1E10 vg/eye, or at least about 1E11 vg/eye, preferably wherein the vector is administered at a dose of about 1E9 vg/eye to about 5E12 vg/eye.

62. The vector or pharmaceutical composition for use according to any of claim 49, 52, or 55-61, the use according to any of claim 50, 53 or 55-61, or the method according to any of claim 51 or 54-61, wherein the vector or pharmaceutical composition is administered in response to relapse of an inflammatory eye disease, preferably wherein the inflammatory eye disease is uveitis.


SEQUENCE LISTING (96 sequences)

SEQ ID NO: 1 - protein, 6 aa, synthetic construct
TFDDYA

SEQ ID NO: 2 - protein, 8 aa, synthetic construct
TWNSGHID

SEQ ID NO: 3 - protein, 10 aa, synthetic construct
VSYLSTASSL

SEQ ID NO: 4 - protein, 7 aa, synthetic construct
GIRNYLA

SEQ ID NO: 5 - protein, 7 aa, synthetic construct
YAASTLQ

SEQ ID NO: 6 - protein, 5 aa, synthetic construct
RYNRA

SEQ ID NO: 7 - protein, 120 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVS

SEQ ID NO: 8 - protein, 107 aa, synthetic construct [sequence omitted]

SEQ ID NO: 9 - protein, 219 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 451 aa, synthetic construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 6 aa, synthetic construct
IFSNHW

SEQ ID NO: 13 - protein, 10 aa, synthetic construct
RSKSINSATH

SEQ ID NO: 14 - protein, 7 aa, synthetic construct
NYYGSTY

SEQ ID NO: 15 - protein, 7 aa, synthetic construct
FVGSSIH

SEQ ID NO: 16 - protein, 7 aa, synthetic construct
KYASESM

SEQ ID NO: 17 - protein, 5 aa, synthetic construct
QSHSW

SEQ ID NO: 18 - protein, 120 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 19 - protein, 107 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 20 - protein, 218 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 21 - protein, 450 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 6 aa, synthetic construct
IFSSYA

SEQ ID NO: 24 - protein, 8 aa, synthetic construct
SYDGSNKK

SEQ ID NO: 25 - protein, 7 aa, synthetic construct
NYYYYGM

SEQ ID NO: 26 - protein, 7 aa, synthetic construct
SVYSYLA

SEQ ID NO: 27 - protein, 7 aa, synthetic construct
YDASNRA

SEQ ID NO: 28 - protein, 5 aa, synthetic construct
QRSNW

SEQ ID NO: 29 - protein, 126 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 30 - protein, 108 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 31 - protein, 224 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 32 - protein, 456 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 222 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLLLECINENNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 6 aa, synthetic construct
VFTDYG

SEQ ID NO: 35 - protein, 8 aa, synthetic construct
NTYIGEPI

SEQ ID NO: 36 - protein, 7 aa, synthetic construct
GYRSYAM

SEQ ID NO: 37 - protein, 7 aa, synthetic construct
NVGTNVA

SEQ ID NO: 38 - protein, 7 aa, synthetic construct
YSASFLY

SEQ ID NO: 39 - protein, 5 aa, synthetic construct
QYNIY

SEQ ID NO: 40 - protein, 118 aa, synthetic construct
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSS

SEQ ID NO: 41 - protein, 107 aa, synthetic construct
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIK

SEQ ID NO: 42 - protein, 216 aa, synthetic construct
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 43 - protein, 229 aa, synthetic construct
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 44 - protein, 214 aa, synthetic construct
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 26 aa, synthetic construct
MATGSRTSLLLAFGLLCLPWLQEGSA

SEQ ID NO: 47 - DNA, 657 bp, synthetic construct
  source 1..657: mol_type = other DNA
gaggtgcaactggtggaaagcggcggcggcctggtccagcctggaaggtccctgagactgagctgtgccgccagcggctttaccttcgacgactacgccatgcactgggtgcgccaggcccctggcaagggcctggaatgggtctccgccatcacctggaatagcggccacatcgactacgccgatagcgtggaaggcagattcaccatcagccgggacaacgccaagaactctctgtatctgcaaatgaacagcctgcgggctgaagatacagccgtgtactattgcgccaaagtgagctacctctccaccgccagcagcctggactattggggacagggcaccctggtgaccgtgtctagcgcctccacaaagggcccttctgtgtttccactggctccaagctccaaaagcacatctggaggaaccgctgccctgggctgcctggttaaggactacttccccgagcctgtgaccgtgagctggaacagcggcgccctgacatctggtgttcataccttccctgccgttctgcaatcttctggactctacagcctgtcttctgtggtgaccgtgcccagcagcagccttggaacacagacctacatctgcaatgtgaaccacaagcctagcaacaccaaggtggacaagaaggtg

SEQ ID NO: 48 - DNA, 642 bp, synthetic construct
  source 1..642: mol_type = other DNA
gacatccagatgacccagagcccttcctcactgagcgccagcgtgggcgacagagtgactattacatgcagagccagccaaggcatccggaactacctggcctggtatcagcagaagcccggcaaagcccctaagctgctgatctacgccgccagcaccctgcaaagcggcgtgcctagcagattcagcggctcaggctctggcactgatttcaccctgaccatctcctctctgcaacctgaggacgtggccacatactactgccagagatacaacagagccccatacacctttggccagggcacaaaagtggaaatcaagagaaccgtggccgctcccagtgtgttcatcttcccccccagtgatgagcagctgaagtccggcacagcctctgtcgtgtgcctgctgaacaacttctaccccagagaggccaaggtgcagtggaaggtggataatgccctgcaaagcggcaacagccaggagagcgtgacagagcaggacagcaaggacagcacctacagcctctctagcacactgaccctgagcaaggccgactacgagaagcacaaggtgtacgcatgcgaggtgacccaccagggcctgagcagtcctgtgaccaagagcttcaaccggggcgagtgt

SEQ ID NO: 49 - DNA, 654 bp, synthetic construct
  source 1..654: mol_type = other DNA
gaggtgaaactggaggaaagtggaggaggcctcgttcaaccaggcggctccatgaagctgtcgtgtgtggcatctggcttcatcttcagcaatcactggatgaactgggtcaggcaatctcctgagaaggggctagagtgggtggcggagatccgctcaaaatcaatcaattccgccacacattatgcagagtcagtaaaagggcggttcaccatttctagagatgacagcaaaagcgccgtgtacctccagatgaccgacctgcgaacagaggacactggggtctactactgctcccggaactactatggctccacctatgactactggggccaagggaccacattgacagtatcctcagcctccactaaaggtccttcagtgtttccgctggctccctcctccaaaagtacgtcaggcggcaccgctgctctgggctgtctggtgaaggattacttccctgaacctgtgactgtttcctggaacagtggagccttgacttcaggagtccacacatttccggcagtgctccagagcagtggtctctattccctaagcagtgtagtgaccgtgccctctagcagcctcggaacccagacatacatctgcaatgtcaatcacaagccaagcaatacaaaagtggacaagaaggtt

SEQ ID NO: 50 - DNA, 642 bp, synthetic construct
  source 1..642: mol_type = other DNA
gacattctcctgacccagtcccctgctatcttgtctgtctcccccggagagcgcgtctccttctcttgcagagcttcccagtttgtgggcagcagcattcactggtatcagcagagaacaaatggatcaccaaggcttttgatcaagtatgcttcagaaagcatgagtgggataccatccaggtttagtggaagtggctctggtactgacttcactctctctataaacacggtggaaagcgaagatattgctgactattactgtcagcaaagccatagctggccatttacttttggatcagggaccaacctggaagtcaagagaactgtggccgcgccttcggtttttattttccccccatctgatgaacagctgaagagcggtacagccagtgtagtgtgcctgctcaacaacttctaccctagagaagccaaggtgcagtggaaggtcgacaatgcattacagagcgggaacagccaggaaagtgttactgagcaggatagcaaggacagcacctactctctgtctagcacactcactttgtctaaagcagattatgagaaacataaagtttatgcctgtgaagttacccaccagggcctgagcagtcccgtcaccaagtctttcaaccgcggggagtgc

SEQ ID NO: 51 - DNA, 77 bp, synthetic construct
  source 1..77: mol_type = other DNA
atggcaacaggtagtcgaaccagcctattactggccttcggtctcctgtgtctgccctggcttcaagagggctctgc

SEQ ID NO: 52 - DNA, 78 bp, synthetic construct
  source 1..78: mol_type = other DNA
atggccacgggaagtcggacttccttactactcgcctttggtcttctttgcttgccatggctccaggagggtagtgca

SEQ ID NO: 53 - DNA, 78 bp, synthetic construct
  source 1..78: mol_type = other DNA
atggccacaggctctcggaccagcctgctgctggccttcggcctgctgtgtctgccttggctgcaagagggcagcgcc

SEQ ID NO: 54 - DNA, 78 bp, synthetic construct
  source 1..78: mol_type = other DNA
atggctaccggcagcagaaccagcctgctgctggcattcggccttctgtgcctgccttggctgcaagagggctctgcc

SEQ ID NO: 55 - protein, 19 aa, synthetic construct
ATNFSLLKQAGDVEENPGP

SEQ ID NO: 56 - protein, 18 aa, synthetic construct
EGRGSLLTCGDVEENPGP

SEQ ID NO: 57 - protein, 20 aa, synthetic construct
QCTNYALLKLAGDVESNPGP

SEQ ID NO: 58 - protein, 22 aa, synthetic construct
VKQTLNFDLLKLAGDVESNPGP

SEQ ID NO: 59 - DNA, 57 bp, synthetic construct
  source 1..57: mol_type = other DNA
gctacgaatttttcattgctcaagcaagcgggagatgtggaggagaaccctggcccc

SEQ ID NO: 60 - DNA, 57 bp, synthetic construct
  source 1..57: mol_type = other DNA
gctaccaacttcagcctcctgaaacaggccggcgatgtggaggaaaaccctggacct

SEQ ID NO: 61 - protein, 27 aa, synthetic construct
RKRRSGSGATNFSLLKQAGDVEENPGP

SEQ ID NO: 62 - DNA, 81 bp, synthetic construct
  source 1..81: mol_type = other DNA
aggaagcggcgttccgggagcggggctacgaatttttcattgctcaagcaagcgggagatgtggaggagaaccctggcccc

SEQ ID NO: 63 - DNA, 81 bp, synthetic construct
  source 1..81: mol_type = other DNA
agaaagcggagaagcggaagcggtgctaccaacttcagcctcctgaaacaggccggcgatgtggaggaaaaccctggacct

SEQ ID NO: 64 - protein, 9 aa, synthetic construct
YPYDVPDYA

SEQ ID NO: 65 - protein, 512 aa, synthetic construct
MATGSRTSLLLAFGLLCLPWLQEGSAEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVRKRRSGSGATNFSLLKQAGDVEENPGPMATGSRTSLLLAFGLLCLPWLQEGSADIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 1536 bp, synthetic construct
  source 1..1536: mol_type = other DNA
atggccacaggctctcggaccagcctgctgctggccttcggcctgctgtgtctgccttggctgcaagagggcagcgccgaggtgcaactggtggaaagcggcggcggcctggtccagcctggaaggtccctgagactgagctgtgccgccagcggctttaccttcgacgactacgccatgcactgggtgcgccaggcccctggcaagggcctggaatgggtctccgccatcacctggaatagcggccacatcgactacgccgatagcgtggaaggcagattcaccatcagccgggacaacgccaagaactctctgtatctgcaaatgaacagcctgcgggctgaagatacagccgtgtactattgcgccaaagtgagctacctctccaccgccagcagcctggactattggggacagggcaccctggtgaccgtgtctagcgcctccacaaagggcccttctgtgtttccactggctccaagctccaaaagcacatctggaggaaccgctgccctgggctgcctggttaaggactacttccccgagcctgtgaccgtgagctggaacagcggcgccctgacatctggtgttcataccttccctgccgttctgcaatcttctggactctacagcctgtcttctgtggtgaccgtgcccagcagcagccttggaacacagacctacatctgcaatgtgaaccacaagcctagcaacaccaaggtggacaagaaggtgagaaagcggagaagcggaagcggtgctaccaacttcagcctcctgaaacaggccggcgatgtggaggaaaaccctggacctatggctaccggcagcagaaccagcctgctgctggcattcggccttctgtgcctgccttggctgcaagagggctctgccgacatccagatgacccagagcccttcctcactgagcgccagcgtgggcgacagagtgactattacatgcagagccagccaaggcatccggaactacctggcctggtatcagcagaagcccggcaaagcccctaagctgctgatctacgccgccagcaccctgcaaagcggcgtgcctagcagattcagcggctcaggctctggcactgatttcaccctgaccatctcctctctgcaacctgaggacgtggccacatactactgccagagatacaacagagccccatacacctttggccagggcacaaaagtggaaatcaagagaaccgtggccgctcccagtgtgttcatcttcccccccagtgatgagcagctgaagtccggcacagcctctgtcgtgtgcctgctgaacaacttctaccccagagaggccaaggtgcagtggaaggtggataatgccctgcaaagcggcaacagccaggagagcgtgacagagcaggacagcaaggacagcacctacagcctctctagcacactgaccctgagcaaggccgactacgagaagcacaaggtgtacgcatgcgaggtgacccaccagggcctgagcagtcctgtgaccaagagcttcaaccggggcgagtgt

SEQ ID NO: 67 - protein, 511 aa, synthetic construct
MATGSRTSLLLAFGLLCLPWLQEGSAEVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVRKRRSGSGATNFSLLKQAGDVEENPGPMATGSRTSLLLAFGLLCLPWLQEGSADILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 1533 bp, synthetic construct
  source 1..1533: mol_type = other DNA
atggcaacaggtagtcgaaccagcctattactggccttcggtctcctgtgtctgccctggcttcaagagggctctgctgaggtgaaactggaggaaagtggaggaggcctcgttcaaccaggcggctccatgaagctgtcgtgtgtggcatctggcttcatcttcagcaatcactggatgaactgggtcaggcaatctcctgagaaggggctagagtgggtggcggagatccgctcaaaatcaatcaattccgccacacattatgcagagtcagtaaaagggcggttcaccatttctagagatgacagcaaaagcgccgtgtacctccagatgaccgacctgcgaacagaggacactggggtctactactgctcccggaactactatggctccacctatgactactggggccaagggaccacattgacagtatcctcagcctccactaaaggtccttcagtgtttccgctggctccctcctccaaaagtacgtcaggcggcaccgctgctctgggctgtctggtgaaggattacttccctgaacctgtgactgtttcctggaacagtggagccttgacttcaggagtccacacatttccggcagtgctccagagcagtggtctctattccctaagcagtgtagtgaccgtgccctctagcagcctcggaacccagacatacatctgcaatgtcaatcacaagccaagcaatacaaaagtggacaagaaggttaggaagcggcgttccgggagcggggctacgaatttttcattgctcaagcaagcgggagatgtggaggagaaccctggccccatggccacgggaagtcggacttccttactactcgcctttggtcttctttgcttgccatggctccaggagggtagtgcagacattctcctgacccagtcccctgctatcttgtctgtctcccccggagagcgcgtctccttctcttgcagagcttcccagtttgtgggcagcagcattcactggtatcagcagagaacaaatggatcaccaaggcttttgatcaagtatgcttcagaaagcatgagtgggataccatccaggtttagtggaagtggctctggtactgacttcactctctctataaacacggtggaaagcgaagatattgctgactattactgtcagcaaagccatagctggccatttacttttggatcagggaccaacctggaagtcaagagaactgtggccgcgccttcggtttttattttccccccatctgatgaacagctgaagagcggtacagccagtgtagtgtgcctgctcaacaacttctaccctagagaagccaaggtgcagtggaaggtcgacaatgcattacagagcgggaacagccaggaaagtgttactgagcaggatagcaaggacagcacctactctctgtctagcacactcactttgtctaaagcagattatgagaaacataaagtttatgcctgtgaagttacccaccagggcctgagcagtcccgtcaccaagtctttcaaccgcggggagtgc

SEQ ID NO: 69 - DNA, 182 bp, synthetic construct
  source 1..182: mol_type = other DNA
agcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 70-74 - skipped (5 entries)

SEQ ID NO: 75 - DNA, 14 bp, synthetic construct
  source 1..14: mol_type = other DNA
ggggactttccact

SEQ ID NO: 76 - DNA, 307 bp, synthetic construct
  source 1..307: mol_type = other DNA
tgagtcactgactcagtgagtcactgactcagtgagtaaggaattctggggactttccactggggactttccactggggactttccactggggactttccactggggactttccactcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttg

SEQ ID NO: 77 - DNA, 2030 bp, synthetic construct
  source 1..2030: mol_type = other DNA
tgagtcactgactcagtgagtcactgactcagtgagtaaggaattctggggactttccactggggactttccactggggactttccactggggactttccactggggactttccactcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggccacaggctctcggaccagcctgctgctggccttcggcctgctgtgtctgccttggctgcaagagggcagcgccgaggtgcaactggtggaaagcggcggcggcctggtccagcctggaaggtccctgagactgagctgtgccgccagcggctttaccttcgacgactacgccatgcactgggtgcgccaggcccctggcaagggcctggaatgggtctccgccatcacctggaatagcggccacatcgactacgccgatagcgtggaaggcagattcaccatcagccgggacaacgccaagaactctctgtatctgcaaatgaacagcctgcgggctgaagatacagccgtgtactattgcgccaaagtgagctacctctccaccgccagcagcctggactattggggacagggcaccctggtgaccgtgtctagcgcctccacaaagggcccttctgtgtttccactggctccaagctccaaaagcacatctggaggaaccgctgccctgggctgcctggttaaggactacttccccgagcctgtgaccgtgagctggaacagcggcgccctgacatctggtgttcataccttccctgccgttctgcaatcttctggactctacagcctgtcttctgtggtgaccgtgcccagcagcagccttggaacacagacctacatctgcaatgtgaaccacaagcctagcaacaccaaggtggacaagaaggtgagaaagcggagaagcggaagcggtgctaccaacttcagcctcctgaaacaggccggcgatgtggaggaaaaccctggacctatggctaccggcagcagaaccagcctgctgctggcattcggccttctgtgcctgccttggctgcaagagggctctgccgacatccagatgacccagagcccttcctcactgagcgccagcgtgggcgacagagtgactattacatgcagagccagccaaggcatccggaactacctggcctggtatcagcagaagcccggcaaagcccctaagctgctgatctacgccgccagcaccctgcaaagcggcgtgcctagcagattcagcggctcaggctctggcactgatttcaccctgaccatctcctctctgcaacctgaggacgtggccacatactactgccagagatacaacagagccccatacacctttggccagggcacaaaagtggaaatcaagagaaccgtggccgctcccagtgtgttcatcttcccccccagtgatgagcagctgaagtccggcacagcctctgtcgtgtgcctgctgaacaacttctaccccagagaggccaaggtgcagtggaaggtggataatgccctgcaaagcggcaacagccaggagagcgtgacagagcaggacagcaaggacagcacctacagcctctctagcacactgaccctgagcaaggccgactacgagaagcacaaggtgtacgcatgcgaggtgacccaccagggcctgagcagtcctgtgaccaagagcttcaaccggggcgagtgt

SEQ ID NO: 78 - DNA, 215 bp, synthetic construct
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 79 - DNA, 542 bp, synthetic construct
  source 1..542: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 80 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 81 - DNA, 25 bp, synthetic construct
  source 1..25: mol_type = other DNA
tttagggttagggttagggttaggg

SEQ ID NO: 82 - DNA, 177 bp, synthetic construct
  source 1..177: mol_type = other DNA
tttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggttttt

SEQ ID NO: 83 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 84 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - protein, 735 aa, synthetic construct [sequence omitted]

SEQ ID NO: 86 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRFLTRNL

SEQ ID NO: 87 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 88 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 89 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAGLSPPTRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 90 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAANNPTPSRAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 91 - DNA, 3446 bp, synthetic construct
  source 1..3446: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctgagtcactgactcagtgagtcactgactcagtgagtaaggaattctggggactttccactggggactttccactggggactttccactggggactttccactggggactttccactcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggccacaggctctcggaccagcctgctgctggccttcggcctgctgtgtctgccttggctgcaagagggcagcgccgaggtgcaactggtggaaagcggcggcggcctggtccagcctggaaggtccctgagactgagctgtgccgccagcggctttaccttcgacgactacgccatgcactgggtgcgccaggcccctggcaagggcctggaatgggtctccgccatcacctggaatagcggccacatcgactacgccgatagcgtggaaggcagattcaccatcagccgggacaacgccaagaactctctgtatctgcaaatgaacagcctgcgggctgaagatacagccgtgtactattgcgccaaagtgagctacctctccaccgccagcagcctggactattggggacagggcaccctggtgaccgtgtctagcgcctccacaaagggcccttctgtgtttccactggctccaagctccaaaagcacatctggaggaaccgctgccctgggctgcctggttaaggactacttccccgagcctgtgaccgtgagctggaacagcggcgccctgacatctggtgttcataccttccctgccgttctgcaatcttctggactctacagcctgtcttctgtggtgaccgtgcccagcagcagccttggaacacagacctacatctgcaatgtgaaccacaagcctagcaacaccaaggtggacaagaaggtgagaaagcggagaagcggaagcggtgctaccaacttcagcctcctgaaacaggccggcgatgtggaggaaaaccctggacctatggctaccggcagcagaaccagcctgctgctggcattcggccttctgtgcctgccttggctgcaagagggctctgccgacatccagatgacccagagcccttcctcactgagcgccagcgtgggcgacagagtgactattacatgcagagccagccaaggcatccggaactacctggcctggtatcagcagaagcccggcaaagcccctaagctgctgatctacgccgccagcaccctgcaaagcggcgtgcctagcagattcagcggctcaggctctggcactgatttcaccctgaccatctcctctctgcaacctgaggacgtggccacatactactgccagagatacaacagagccccatacacctttggccagggcacaaaagtggaaatcaagagaaccgtggccgctcccagtgtgttcatcttcccccccagtgatgagcagctgaagtccggcacagcctctgtcgtgtgcctgctgaacaacttctaccccagagaggccaaggtgcagtggaaggtggataatgccctgcaaagcggcaacagccaggagagcgtgacagagcaggacagcaaggacagcacctacagcctctctagcacactgaccctgagcaaggccgactacgagaagcacaaggtgtacgcatgcgaggtgacccaccagggcctgagcagtcctgtgaccaagagcttcaaccggggcgagtgttacccatacgatgttccagattacgcttgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggtacctttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggtttttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 92 - DNA, 3443 bp, synthetic construct
  source 1..3443: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctgagtcactgactcagtgagtcactgactcagtgagtaaggaattctggggactttccactggggactttccactggggactttccactggggactttccactggggactttccactcctgcaggagcttgaataaaatgaatattagaagctgttagaataagagaaaatgacagaggaaaactgaaagggagaactgaaagtgggaaattcctctgaggcagaaaggaccatcccttataaatagcacaggccatgaaggaagatcattctcactgcagcctttgacagcctttgcctcatcttgcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcaacaggtagtcgaaccagcctattactggccttcggtctcctgtgtctgccctggcttcaagagggctctgctgaggtgaaactggaggaaagtggaggaggcctcgttcaaccaggcggctccatgaagctgtcgtgtgtggcatctggcttcatcttcagcaatcactggatgaactgggtcaggcaatctcctgagaaggggctagagtgggtggcggagatccgctcaaaatcaatcaattccgccacacattatgcagagtcagtaaaagggcggttcaccatttctagagatgacagcaaaagcgccgtgtacctccagatgaccgacctgcgaacagaggacactggggtctactactgctcccggaactactatggctccacctatgactactggggccaagggaccacattgacagtatcctcagcctccactaaaggtccttcagtgtttccgctggctccctcctccaaaagtacgtcaggcggcaccgctgctctgggctgtctggtgaaggattacttccctgaacctgtgactgtttcctggaacagtggagccttgacttcaggagtccacacatttccggcagtgctccagagcagtggtctctattccctaagcagtgtagtgaccgtgccctctagcagcctcggaacccagacatacatctgcaatgtcaatcacaagccaagcaatacaaaagtggacaagaaggttaggaagcggcgttccgggagcggggctacgaatttttcattgctcaagcaagcgggagatgtggaggagaaccctggccccatggccacgggaagtcggacttccttactactcgcctttggtcttctttgcttgccatggctccaggagggtagtgcagacattctcctgacccagtcccctgctatcttgtctgtctcccccggagagcgcgtctccttctcttgcagagcttcccagtttgtgggcagcagcattcactggtatcagcagagaacaaatggatcaccaaggcttttgatcaagtatgcttcagaaagcatgagtgggataccatccaggtttagtggaagtggctctggtactgacttcactctctctataaacacggtggaaagcgaagatattgctgactattactgtcagcaaagccatagctggccatttacttttggatcagggaccaacctggaagtcaagagaactgtggccgcgccttcggtttttattttccccccatctgatgaacagctgaagagcggtacagccagtgtagtgtgcctgctcaacaacttctaccctagagaagccaaggtgcagtggaaggtcgacaatgcattacagagcgggaacagccaggaaagtgttactgagcaggatagcaaggacagcacctactctctgtctagcacactcactttgtctaaagcagattatgagaaacataaagtttatgcctgtgaagttacccaccagggcctgagcagtcccgtcaccaagtctttcaaccgcggggagtgctacccatacgatgttccagattacgcttgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggtacctttagggttagggttagggttagggaaaaatttagggttagggttagggttagggaaaaatttagggttagggttagggttagggaaaaatgcagcggtaagttcccatccaggttttttgcagcggtaagttcccatccaggttttttgcagcggtaagttcccatccaggtttttctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 93 - protein, 438 aa, synthetic construct
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 725 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMLAHKFKSGDAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 95 - DNA, 10 bp, synthetic construct
  source 1..10: mol_type = other DNA
gccrccatgg

SEQ ID NO: 96 - DNA, 13 bp, synthetic construct
  source 1..13: mol_type = other DNA
gccgccrccatgg